(19) United States
(12) Patent Application Publication
(10) Pub. No.: US 2026/0369343 A1
(43) Pub. Date: Nov. 19, 2026

(54) DIAGNOSIS OF OVARIAN CANCER USING TARGETED QUANTIFICATION OF SITE-SPECIFIC PROTEIN GLYCOSYLATION

(71) Applicant: VENN BIOSCIENCES CORPORATION, South San Francisco, CA (US)

(72) Inventors: Chirag DHAR, South San Francisco, CA (US); Prasanna RAMACHANDRAN, South San Francisco, CA (US); Tomislav CAVAL, South San Francisco, CA (US)

(21) Appl. No.: 19/110,885

(22) PCT Filed: Sep. 14, 2023
(86) PCT No.: PCT/US2023/074251

§ 371 Date: Mar. 12, 2025

Related U.S. Application Data
(60) Provisional application No. 63/376,053, filed on Sep. 16, 2022.
(60) Provisional application No. 63/489,712, filed on Mar. 10, 2023.
(60) Provisional application No. 63/517,859, filed on Aug. 4, 2023.

(51) Int. Cl. G16B 15/20; G01N 1/40; G01N 27/62; G01N 33/68; G16B 25/10; G16B 40/20; G16H 10/40; G16H 10/60; G16H 20/00; G16H 50/20; G16H 50/30

(52) U.S. Cl. CPC G16B 15/20; G01N 1/4044; G01N 27/62; G01N 33/6842; G01N 33/6848; G16B 25/10; G16B 40/20; G16H 10/40; G16H 10/60; G16H 20/00; G16H 50/20; G16H 50/30

(57) ABSTRACT

A method and system for diagnosing a subject with respect to an ovarian cancer disease state. Peptide structure data corresponding to a biological sample obtained from the subject is received. The peptide structure data is analyzed using a supervised machine learning model to generate a disease indicator that indicates whether biological sample evidences the ovarian cancer disease state based on at least 1 peptide structures selected from a group of peptide structures identified in Table 3B, 3C, or 3D. The group of peptide structures in Table 3B, 3C, or 3D comprises a group of peptide structures associated with the ovarian cancer disease state. A diagnosis output is generated based on the disease indicator.

This application claims priority to U.S. Provisional Patent Application Ser. No. 63/376,053, filed Sep. 16, 2022, and claims priority to U.S. Provisional Patent Application Ser. No. 63/489,712, filed Mar. 10, 2023, and claims priority to U.S. Provisional Patent Application Ser. No. 63/517,859, filed Aug. 4, 2023, all of which applications are incorporated by reference herein in their entirety.

### FIELD

Embodiments of the present disclosure generally relate to methods and systems for analyzing peptide structures for diagnosing and/or treating ovarian cancer. More particularly, embodiments of the present disclosure relate to analyzing quantification data for a set of peptide structures detected in a biological sample obtained from a subject for use in diagnosing and/or treating the subject, the set of peptide structures being associated with ovarian cancer.

### BACKGROUND

Protein glycosylation and other post-translational modifications play vital roles in virtually all aspects of human physiology. Unsurprisingly, faulty or altered protein glycosylation often accompanies various disease states. The identification of aberrant glycosylation provides opportunities for early detection, intervention, and treatment of affected subjects. Current biomarker identification methods, such as those developed in the fields of proteomics and genomics, can be used to detect indicators of certain diseases, such as cancer, and to differentiate certain types of cancer from other, non-cancerous diseases. However, the use of glycoproteomic analyses has not previously been used to successfully identify disease processes.

Glycoprotein analysis is fraught with challenges on several levels. For example, a single glycan composition in a peptide can contain a large number of isomeric structures due to different glycosidic linkages, branching patterns, and/or multiple monosaccharides having the same mass. In addition, the presence of multiple glycans that share the same peptide backbone can lead to assay signals from various glycoforms, lowering their individual abundances compared to aglycosylated peptides. Accordingly, the development of algorithms that can identify glycan structures on peptide fragments remains elusive.

In light of the above, there is a need for improved analytical methods that involve site-specific analysis of glycoproteins to obtain information about protein glycosylation patterns, which can in turn provide quantitative information that can be used to identify disease states. For example, there is a need to use such analysis to diagnose and/or treat ovarian cancer.

Epithelial ovarian cancer (EOC) is currently the second-most common gynecologic malignancy, the leading cause of death from gynecological cancer, and the fourth-leading cause of cancer-related death in women in the United States. Although EOC can be treated effectively with surgery and adjuvant therapies, only about 15-20% of women are diagnosed at early-stage when 5-year survival is greater than 90%. Instead, the majority of EOC cases are diagnosed at late-stage (stage III or IV), with 5-year survival rates between about 15% and 40%. Diagnosing early-stage EOC is impeded by initial clinical signs and symptoms that are generally nonspecific and commonly missed such as, for example, pelvic pain, urinary urgency/frequency, abdominal bloating, early satiety, loss of appetite, and weight loss.

In addition to late diagnosis and consequent under-treatment of serious disease, benign disease is oftentimes unnecessarily over-treated due to the lack of diagnostic tools to determine the nature of pelvic masses. For example, while over 90% of women presenting with a pelvic mass may ultimately undergo surgery, only about 20% are found to have malignant disease.

Thus, an approach that is non-invasive, accurate, and reliable and that enables early diagnosis is needed. An approach enabling early diagnosis may help reduce negative health outcomes in patients with ovarian cancer, reduce the under-treatment of ovarian cancer, and/or reduce the over-treatment of benign disease. In addition, more strategic treatments can be provided with a diagnostic test that can assess whether a subject has early stage or late stage ovarian cancer. Thus, it may be desirable to have methods and systems capable of addressing one or more of the above-identified issues.

### SUMMARY

In an embodiment, a method for diagnosing a subject with respect to an ovarian cancer disease state is described. The method includes receiving peptide structure data corresponding to a biological sample obtained from the subject. The peptide structure data can be analyzed using a supervised machine learning model to generate a disease indicator that indicates whether the biological sample evidences the ovarian cancer disease state of having early stage or late stage ovarian cancer based on at least one peptide structure selected from one of a group of peptide structures identified in Tables 3B, 3C, or 3D. A diagnosis output can be generated based on the disease indicator. The disease indicator can include a score.

The method of generating the diagnosis output can include determining that the score falls above a selected threshold and generating the diagnosis output based on the score falling above the selected threshold, wherein the diagnosis output includes a classification of late stage ovarian cancer disease state. The method of generating the diagnosis output can include determining that the score falls below a selected threshold and generating the diagnosis output based on the score falling below the selected threshold, wherein the diagnosis output includes a classification of early stage ovarian cancer disease state. The score may include a probability score and the selected threshold is 0.5. Alternatively, the selected threshold may fall within a range between 0.30 and 0.65. In an embodiment, the analyzing the peptide structure data can include analyzing the peptide structure data using a binary classification model. The peptide structure of the at least one peptide structure can include a glycopeptide structure defined by a peptide sequence and a glycan structure linked to the peptide sequence at a linking site of the peptide sequence, as identified in Table 3D, with the peptide sequence being one of SEQ ID NOS: 126-175 in Table 3D as defined in Table 5.

In another embodiment, the method can include training the supervised machine learning model using training data, wherein the training data comprises a plurality of peptide structure profiles for a plurality of subjects and a plurality of subject diagnoses for the plurality of subjects, wherein the plurality of subject diagnoses includes a diagnosis for any subject of the plurality of subjects determined to have early stage or late stage ovarian cancer.

In another embodiment, the method can include performing a differential expression analysis using initial training data to compare a first portion of the plurality of subjects diagnosed with the classification of early stage ovarian cancer disease state versus a second portion of the plurality of subjects diagnosed with the classification of late stage ovarian cancer disease state; identifying a training group of peptide structures based on the differential expression analysis for use as prognostic markers for the ovarian cancer disease state; and forming the training data based on the training group of peptide structures identified. The training of the supervised machine learning model can include reducing the training group of peptide structures to a final group of peptide structures identified in Tables 3B, 3C, or 3D.

In an embodiment, each peptide structure profile of the plurality of peptide structure profiles can include a feature selected from one of a relative abundance and a concentration for a corresponding peptide structure. The plurality of peptide structure profiles can include a first peptide structure profile with a relative abundance for a corresponding peptide structure and a second peptide structure profile with a concentration for the corresponding peptide structure. The supervised machine learning model can include a logistic regression model.

In an embodiment, the first group of peptide structures in Tables 3B, 3C, or 3D is used to distinguish between the ovarian cancer disease state being late stage or early stage. The quantification data for a peptide structure of the set of peptide structures can include at least one of an abundance, a relative abundance, a normalized abundance, a relative quantity, an adjusted quantity, a normalized quantity, a relative concentration, an adjusted concentration, or a normalized concentration.

In an embodiment, the peptide structure data can be generated using multiple reaction monitoring mass spectrometry (MRM-MS), wherein the using of the MRM-MS includes ionizing one or more glycopeptides to form ionized glycopeptides; filtering the ionized glycopeptides with a mass filter to form filtered glycopeptides; fragmenting the filtered glycopeptides in a collision chamber into product ions; and detecting the product ions.

In an embodiment, the method can include preparing a sample of the biological sample using reduction, alkylation, and enzymatic digestion to form a prepared sample that includes a set of peptide structures.

In an embodiment, the method of classifying early and late stage ovarian cancer can be implemented after the subject has already been diagnosed as having ovarian cancer. The subject can be initially diagnosed for having ovarian cancer using one or more biomarkers in Tables 1, 2, or 3.

In an embodiment, the generating the diagnosis output can include generating a report identifying that the biological sample evidences the early stage or late stage ovarian cancer disease state.

In an embodiment, the generating a treatment output can be generated based on at least one of the diagnosis output or the disease indicator. The treatment output can include at least one of an identification of a treatment to treat the subject or a treatment plan. The treatment can include at least one of surgery, radiation therapy, a targeted drug therapy, chemotherapy, immunotherapy, hormone therapy, or neoadjuvant therapy. In some embodiments, the group of peptide structures in Tables 3B, 3C, or 3D is listed in order of relative significance to the disease indicator.

In an embodiment, the method can further include preparing a sample of the biological sample using reduction, alkylation, and enzymatic digestion to form a prepared sample that includes a set of peptide structures. The method can further include generating the peptide structure data from the prepared sample using multiple reaction monitoring mass spectrometry (MRM-MS).

In an embodiment, a method of training a model to diagnose a subject with respect to an ovarian cancer disease state having a malignant pelvic tumor is described. The method can include receiving quantification data for a panel of peptide structures for a plurality of samples for a plurality of subjects. The plurality of subjects includes a first portion diagnosed with a classification of early stage ovarian cancer disease state and a second portion diagnosed with a classification of late stage ovarian cancer disease state. The quantification data can include a plurality of peptide structure profiles for the plurality of subjects and training a machine learning model using the quantification data to diagnose a biological sample with respect to the ovarian cancer disease state using a group of peptide structures associated with the ovarian cancer disease state, wherein the group of peptide structures is identified in Tables 3B, 3C, or 3D. The machine learning model can include a logistic regression model.

The method of training the model can further include identifying an initial plurality of peptide structure profiles, filtering the initial plurality of peptide structure profiles by a coefficient of variation to generate a plurality of peptide structure profiles for use in training the machine learning model. The filtering can be performed to exclude peptide structure profiles having the coefficient of variation at or above 20%. The training of the machine learning model can include reducing the plurality of peptide structure profiles using LASSO regression to identify a final group of peptide structures identified in Tables 3B, 3C, or 3D. The quantification data for the panel of peptide structures for the plurality of subjects diagnosed with the plurality of ovarian cancer disease states can include at least one of an abundance, a relative abundance, a normalized abundance, a relative quantity, an adjusted quantity, a normalized quantity, a relative concentration, an adjusted concentration, or a normalized concentration. The trained model can use a relative abundance for a first portion of the first group of peptide structures and a concentration for a second portion of the second group of peptide structures. Each peptide structure profile of the plurality of peptide structure profiles includes a feature selected from one of a relative abundance and a concentration for a corresponding peptide structure. The plurality of peptide structure profiles can include a first peptide structure profile with a relative abundance for a corresponding peptide structure and a second peptide structure profile with a concentration for the corresponding peptide structure.

In an embodiment, a composition can include at least one of peptide structures identified in Tables 3B, 3C, or 3D.

In an embodiment, a method for diagnosing a subject with respect to an ovarian cancer disease state is described. The method can include analyzing the peptide structure data using a supervised machine learning model to generate a disease indicator that indicates whether a biological sample evidences the ovarian cancer disease state of having early stage or late stage ovarian cancer based on a group of glycopeptide structures. The group of glycopeptide structures can include tri-antennary or tetra-antennary sialic acid moieties, wherein a portion of the glycopeptide structures of the group are fucosylated. A diagnosis is then outputted based on the disease indicator. The group of glycopeptide structures can include at least one, at least three, at least five, or at least 10 glycopeptide structure identified in Tables 3B, 3C, or 3D.

In an embodiment, the peptide structure data was generated with a mass spectrometer using the biological sample obtained from the subject.

In an embodiment, the method can further include preparing a sample of the biological sample using reduction, alkylation, and enzymatic digestion to form a prepared sample that includes a set of peptide structures. The peptide structure data can be generated from the prepared sample using multiple reaction monitoring mass spectrometry (MRM-MS). The use of the MRM-MS can include ionizing one or more glycopeptides to form ionized glycopeptides; filtering the ionized glycopeptides with a mass filter to form filtered glycopeptides; fragmenting the filtered glycopeptides in a collision chamber into product ions; and detecting the product ions.

In one or more embodiments, a system comprising one or more data processors is described according to various embodiments. In various embodiments, the system comprises a non-transitory computer readable storage medium containing instructions which, when executed on the one or more data processors, cause the one or more data processors to perform part or all of any of the methods described herein.

In one or more embodiments, a computer-program product tangibly embodied in a non-transitory machine-readable storage medium, including instructions configured to cause one or more data processors to perform part or all of any one of the methods described according to various embodiments.

In one or more embodiments, a system is described according to various embodiments. In various embodiments, the system comprises one or more data processors and a non-transitory computer readable storage medium containing instructions which, when executed on the one or more data processors, cause the one or more data processors to perform part or all of any one or more of the methods described herein.

In one or more embodiments, a computer-program product tangibly embodied in a non-transitory machine-readable storage medium, including instructions configured to cause one or more data processors to perform part or all of any one or more of the methods described herein.

In various embodiments, the peptide structure data is listed in Table 3D and the detected product ion comprises a first product having a m/z value listed in Table 4C.

In some embodiments, the at least one peptide structure comprises a peptide sequence and a glycan structure, wherein the glycan structure is attached to a linking site position in the peptide sequence in accordance with one of Tables 1, 2, 3, 3B, 3C, and 3D. In some embodiments, the glycan structure of the peptide sequence corresponds to a glycan structure GL number in accordance with Tables 1, 2, 3, 3B, 3C, and 3D, wherein the glycan structure comprises a symbol structure in accordance with the glycan structure GL number according to Tables 1, 2, 3, 3B, 3C, 3D, and 7. In some embodiments, the glycan structure of the peptide sequence corresponds to a glycan structure GL number in accordance with Tables 1, 2, 3, 3B, 3C, and 3D, wherein the glycan structure comprises a composition in accordance with the glycan structure GL number, Tables 1, 2, 3, 3B, 3C, 3D, and 7. In some embodiments, a rightmost N-acetylgalactosamine (open square) of the glycan structure in Table 7 is attached to a linking site position in the peptide sequence in accordance with Tables 3 and 5. In some embodiments, a bottommost N-acetylglucosamine (dark square) of the glycan structure in Table 7 is attached to a linking site position in the peptide sequence in accordance with Tables 1, 2, 3, 3B, 3C, 3D, and 5.

In some embodiments, provided herein is a composition comprising one or more peptide structures from Tables 1, 2, 3, 3B, 3C, and 3D. In some embodiments, the at least one peptide structure comprises a peptide sequence and a glycan structure, wherein the glycan structure is attached to a linking site position in the peptide sequence in accordance with Tables 1, 2, 3, 3B, 3C, and 3D. In some embodiments, the glycan structure of the peptide sequence corresponds to a glycan structure GL number in accordance with Tables 1, 2, 3, 3B, 3C, and 3D, wherein the glycan structure comprises a symbol structure in accordance with the glycan structure GL number according to Tables 1, 2, 3, 3B, 3C, 3D, and 7. In some embodiments, the glycan structure of the peptide sequence corresponds to a glycan structure GL number in accordance with Tables 1, 2, 3, 3B, 3C, and 3D, wherein the glycan structure comprises a composition in accordance with the glycan structure GL number, Tables 1, 2, 3, 3B, 3C, 3D, and 7. In some embodiments, a rightmost N-acetylgalactosamine (GalNAc) of the glycan structure in Table 7 is attached to a linking site position in the peptide sequence in accordance with Tables 3 and 5. In some embodiments, a bottommost N-acetylglucosamine (GlcNAc) of the glycan structure in Table 7 is attached to a linking site position in the peptide sequence in accordance with Tables 1, 2, 3, 3B, 3C, 3D, and 5.

In regard to the various embodiments, the peptide sequence can be one of SEQ ID NOS: 130-135, 137, 139, 140, 143, 148, 149, 155, 158-162, 166, and 171.

In regards to the various embodiments, the peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 130-135, 137, 139, 140, 143, 148, 149, 155, 158-162, 166, and 171 in Table 3D as defined in Table 5.

In regard to the various embodiments, the peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 130-135, 137, 139, 140, 143, 148, 149, 155, 159-162, 166, and 171 in Table 3D as defined in Table 5.

In regard to the various embodiments, the glycan structure, corresponding to the peptide sequence of SEQ ID NOS: 131, 137, 143, 155, 159, 162, 166, and 171 includes a fucose and the fucose is in an outer arm orientation.

In regard to the various embodiments, a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 131, 137, 143, 155, 159, 162, 166, and 171 in Table 3D as defined in Table 5, wherein a fucose of the glycan structure comprises an outer arm orientation.

In regard to the various embodiments, the at least one peptide structure is selected from one of a group of peptide structures identified in Tables 3D.

In regard to the various embodiments, a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 131-134, 137, 139, 140, 143, 151, 165-167 in Table 3D as defined in Table 5.

In regard to the various embodiments, the glycan structure, corresponding to the peptide sequence of SEQ ID NOS: 131, 137, and 143, includes a fucose and the fucose is in an outer arm orientation.

In regard to the various embodiments, the outer arm orientation of the fucose comprises the fucose being linked to a N-acetylglucosamine by a α-(1-3/4) linkage.

In an embodiment, there is a method of treating ovarian cancer in an individual, the method comprising administering to the individual an ovarian cancer therapy, wherein the individual has been determined to be responsive to the ovarian cancer therapy via a trained machine learning classifier that distinguishes between responsive and non-responsive individuals who have received the ovarian cancer therapy, based at least in part on a group of peptide structures identified in Tables 3B, 3C, or 3D.

### BRIEF DESCRIPTION OF THE DRAWINGS

The present disclosure is described in conjunction with the appended figures:

FIG. 1 is a schematic diagram of an exemplary workflow 100 for the detection of peptide structures associated with a disease state for use in diagnosis and/or treatment in accordance with one or more embodiments.

FIG. 2A is a schematic diagram of a preparation workflow in accordance with one or more embodiments.

FIG. 2B is a schematic diagram of data acquisition in accordance with one or more embodiments.

FIG. 3 is a block diagram of an analysis system in accordance with one or more embodiments.

FIG. 4 is a block diagram of a computer system in accordance with various embodiments.

FIG. 5 is a flowchart of a process for diagnosing a subject with respect to an ovarian cancer disease state in accordance with one or more embodiments based on Tables 1 or 2.

FIG. 6 is a flowchart of a process for diagnosing a subject with respect to ovarian cancer disease state in accordance with one or more embodiments based on Table 3.

FIG. 6B is a flowchart of a process for diagnosing a subject with respect to ovarian cancer disease state in accordance with one or more embodiments based on Table 3B.

FIG. 7 is a flowchart of a process for training a model to diagnose a subject with respect to an ovarian cancer disease state in accordance with one or more embodiments.

FIG. 8 is a table describing the distribution of the samples acquired in this exemplary retrospective analysis in accordance with one or more embodiments.

FIG. 9 is a plot diagram illustrating the results of a principal component analysis performed to assess the segregation between healthy, benign pelvic tumor, and EOC samples across first and second principal components in accordance with one or more embodiments.

FIG. 10 is a plot diagram illustrating the results of a principal component analysis performed to assess segregation between healthy, benign pelvic tumor, early EOC, late EOC, and missing (undocumented) samples).

FIG. 11 is an illustration of a receiver operating characteristic (ROC) diagram corresponding to the multivariable model built to predict malignancy v. benign status of pelvic tumors in accordance with one or more embodiments.

FIG. 12 is an illustration of a diagram showing the probability distributions for the various groups using the multivariable model for predicting malignancy v. benign status of pelvic tumors in accordance with one or more embodiments.

FIG. 13 is an illustration of a receiver operating characteristic (ROC) diagram corresponding to the multivariable model built to predict malignancy v. benign status of pelvic tumors in accordance with one or more embodiments.

FIG. 14 is an illustration of a diagram showing the probability distributions for the various groups using the multivariable model for predicting malignancy v. benign status of pelvic tumors in accordance with one or more embodiments.

FIG. 15A to 15E are a plurality of charts illustrating the upregulation of fucosylated biomarkers having tri or tetra-antennary sialic acids from stages 1/2 to 3/4 of ovarian cancer and the down regulation of non-fucosylated biomarkers having tri or tetra-antennary sialic acids from stages 1/2 to 3/4 of ovarian cancer.

FIG. 16 is an illustration of a diagram showing the probability distributions for early stage v. late stage ovarian cancer using training data set and the testing data set in accordance with one or more embodiments using the biomarkers of Table 3C.

FIG. 17 is an illustration of a receiver operating characteristic (ROC) diagram corresponding to the multivariable model built to predict early stage v. late stage ovarian cancer in accordance with one or more embodiments.

FIG. 18 is a graph illustrating the fold changes for a plurality of tri- and tetra-antennary glycans glycopeptides that were either non-fucosylated or fucosylated.

FIG. 19A is a graph illustrating the fold changes for pairs of tri- and tetra-antennary glycans glycopeptides that were either non-fucosylated or fucosylated.

FIG. 19B is a graph illustrating the fold changes for triplets of tri- and tetra-antennary glycans glycopeptides that were either non-fucosylated, mono-fucosylated, or di-fucosylated. Both mono-fucosylated and di-fucosylated markers has median FC's above 1 suggesting correlation of these markers with malignant EOC.

FIG. 20 is an illustration of a diagram showing the probability distributions for early stage v. late stage ovarian cancer using training data set and the testing data set in accordance with one or more embodiments using the biomarkers of Table 3D

FIGS. 21A to 21E are graphs of the relative abundance of five distinct types of fucosylated glycopeptides in benign tumors, early stage EOC, and late stage EOC.

FIG. 22 is a representative mass spectra showing breakdown fragments of 3 glycans and 4 glycan aggregates that indicate the presence of glycans with an outer arm fucosylated orientation.

### DETAILED DESCRIPTION

### I. Overview

The embodiments described herein recognize that glycoproteomics is an emerging field that can be used in the overall diagnosis and/or treatment of subjects with various types of diseases. Glycoproteomics aims to determine the positions, identities, and quantities of glycans and glycosylated proteins in a given sample (e.g., blood sample, cell, tissue, etc.). Protein glycosylation is one of the most common and most complex forms of post-translational protein modification, and can affect protein structure, conformation, and function. For example, glycoproteins may play crucial roles in important biological processes such as cell signaling, host-pathogen interactions, and immune response and disease. Glycoproteins may therefore be important to diagnosing different types of diseases.

Although protein glycosylation provides useful information about cancer and other diseases, analysis of protein glycosylation may be difficult as the glycan typically cannot be traced back to the protein site of origin with currently available methodologies. Glycoprotein analysis can be challenging in general due to several reasons. For example, a single glycan composition in a peptide may contain a large number of isomeric structures because of different glycosidic linkages, branching, and many monosaccharides having the same mass. Further, the presence of multiple glycans that share the same peptide sequence may cause the mass spectrometry (MS) signal to split into various glycoforms, lowering their individual abundances compared to the peptides that are not glycosylated (aglycosylated peptides).

But to understand various disease conditions and to diagnose certain diseases, such as ovarian cancer, more accurately, it may be important to perform analysis of glycoproteins and to identify not only the glycan but also the linking site (e.g., the amino acid residue of attachment) within the protein. Thus, there is a need to provide a method for site-specific glycoprotein analysis to obtain detailed information about protein glycosylation patterns which may be able to provide information about a disease state (e.g., an ovarian cancer disease state). This information can be used to distinguish the disease state from other states, diagnose a subject as having or not having the disease state, determine a likelihood that a subject has the disease state, determine whether a subject has one of early stage (stages 1 and 2) or late stage (stages 3 and 4) EOC, or a combination thereof. For example, such analysis may be useful in diagnosing an ovarian cancer disease state for a subject (e.g., a negative diagnosis for the ovarian cancer disease state or a positive diagnosis for the ovarian cancer disease state). Sample collection and analysis can be collected at different time points for comparing ovarian cancer disease states over time for a subject. For example, the negative diagnosis may include a healthy state or a benign tumor state (i.e., “benign” as seen throughout). An example of the positive diagnosis includes the subject suffering from a form of ovarian cancer (e.g., epithelial ovarian cancer (EOC)). A diagnosis can also assess a malignancy status of a previously identified pelvic (or adnexal) tumor (or mass).

Accordingly, the embodiments described herein provide various methods and systems for analyzing proteins in subjects and, in particular, glycoproteins. In one or more embodiments, a machine learning model is trained to analyze peptide structure data and generate a disease indicator that provides information relating to one or more diseases. For example, in various embodiments, the peptide structure data comprises quantification metrics (e.g., abundance or concentration data) for peptide structures. A peptide structure may be defined by an aglycosylated peptide sequence (e.g., a peptide or peptide fragment of a larger parent protein) or a glycosylated peptide sequence. A glycosylated peptide sequence (also referred to as a glycopeptide structure) may be a peptide sequence having a glycan structure that is attached to a linking site (e.g., an amino acid residue) of the peptide sequence, which may occur via, for example, a particular atom of the amino acid residue). Non-limiting examples of glycosylated peptides include N-linked glycopeptides and O-linked glycopeptides.

The embodiments described herein recognize that the abundance of selected peptide structures in a biological sample obtained from a subject may be used to determine the likelihood of that subject evidencing an ovarian cancer disease state. An ovarian cancer disease state may include any condition that can be diagnosed as cancer that occurs in in the ovaries. Many malignant pelvic tumors are ovarian cancer. Certain peptide structures that are associated with an ovarian cancer disease state may be more relevant to that disease state than other peptide structures that are also associated with that disease state.

Analyzing the abundance of peptide sequences and glycosylated peptide sequences in a biological sample may provide a more accurate way in which to distinguish a positive ovarian cancer disease state (e.g., a state including the presence of ovarian cancer) from a negative ovarian cancer disease state (e.g., healthy state, a benign tumor state, an absence of ovarian cancer, etc.). This type of peptide structure analysis may be more conducive to generating accurate diagnoses as compared to glycoprotein analysis that focuses on analyzing glycoproteins that are too large to be resolved via mass spectrometry. Further, with glycoproteins, there may be too many potential proteoforms to consider. Still further, analysis of peptide structure data in the manner described by the various embodiments herein may be more conducive to generating accurate diagnoses as compared to glycomic analysis that provides little to no information about what proteins and to which amino acid residue sites various glycan structures attach.

In many instances, ovarian cancer treated with surgical resection will reoccur due to the metastasis. Thus, there is a need for tests that can diagnose metastatic ovarian cancer and monitor the progression of the disease (e.g., assessing the state of early vs late stage ovarian cancer). Such a test may be based on either ELISA or mass spectrometry.

For reference, in stage 1, the cancer is confined to the ovaries and hasn't spread to the abdomen, pelvis or lymph nodes, nor to distant sites. In stage 2, the cancer has spread from one or both ovaries to other areas of the pelvis. However, the cancer hasn't spread to nearby lymph nodes or distant sites. Stages 1 and 2 are considered early stage. In stage 3, the cancer has spread to nearby lymph nodes and/or other parts of the abdomen, but it hasn't spread to distant sites. In stage 4, the cancer has spread beyond the abdomen. Stages 3 and 4 are considered late stage.

A particular type of glycopeptides having fucosylation was found through mass spectrometry measurements to be associated with metastatic ovarian cancer. In addition, this type of glycopeptide had tri- and tetra-antennary N-glycans on certain proteins. In an embodiment, various proteins such as AGP1, AGP2, APOC3, FETUA, HPT, CLUS, A2MG, TRFE, VTNC, IGJ, and CFAH can be captured on an ELISA plate from patient samples followed by a lectin based detection (four lectins: LCA, AAL, PHA-E, PHA-L).

Mass spectrometry can be used to analyze serum for various glycoproteins and/or glycopeptides to differentiate between benign and malignant adnexal masses. Through analyzing the clinical mass spectrometry data, a distinct signature was found with the circulating N-glycoproteins that allows a differentiation between late stage (metastatic disease of stage III/IV) and early stage (stage I/II) epithelial ovarian cancer (EOC). Using Qiagen's Ingenuity Pathway Analysis package on this data, it was predicted that the signature markers are downstream of cytokine signaling. The markers also suggest the presence of the sialyl Lewis X (sLex) epitope on N-glycans of certain liver-derived circulatory glycoproteins. Given these findings suggesting the presence of sLex epitopes in circulation, it was investigated whether the outer-arm fucosylation was upregulated on the tumor itself. Bulk RNASeq data showed the outer-arm fucosyltransferases FUT3, FUT4, and FUT9 were found to be upregulated in late stage EOC. The core fucosyltransferase FUT8 on the other hand was unchanged between early and late stage EOC. A blood-based test would be useful for staging/treatment recommendations and to preempt recurrence and metastatic transformation of epithelial ovarian cancer.

Further, the methods, systems, and compositions provided by the embodiments described herein may enable an earlier and more accurate diagnosis of ovarian cancer in a subject as compared to currently available diagnostic modalities (e.g., imaging, biochemical tests) used for determining whether surgical intervention is indicated. For example, various currently available non-invasive tests to distinguish between benign and malignant pelvic tumors rely on detection of the biomarker cancer antigen 125 (CA125). But this biomarker is limited by poor sensitivity and specificity. In fact, serum CA125 is not elevated in over 20% of ovarian carcinomas and is elevated in a variety of other malignant and non-malignant conditions. While various other tests incorporate other protein biomarkers in addition to CA125, these other tests may perform less adequately than desired and may be more complex than desired. The embodiments described herein enable more reliable prediction of the malignant or benign nature of pelvic (or adnexal) tumors (or masses).

The description below provides exemplary implementations of the methods and systems described herein for the research, diagnosis, and/or treatment of an ovarian cancer disease state. Various examples implement the methods and systems described herein as a screening tool. Descriptions and examples of various terms, as used herein, are provided in Section II below.

### II. Exemplary Descriptions of Terms

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “amino acid,” as used herein, generally refers to any organic compound that includes an amino group (e.g., -NH2), a carboxyl group (—COOH), and a side chain group (R) which varies based on a specific amino acid. Amino acids can be linked using peptide bonds.

The term “alkylation,” as used herein, generally refers to the transfer of an alkyl group from one molecule to another. In various embodiments, alkylation is used to react with reduced cysteines to prevent the re-formation of disulfide bonds after reduction has been performed.

The term “linking site” or “glycosylation site” as used herein generally refers to the location where a sugar molecule of a glycan or glycan structure is directly bound (e.g., covalently bound) to an amino acid of a peptide, a polypeptide, or a protein. For example, the linking site may be an amino acid residue and a glycan structure may be linked via an atom of the amino acid residue. Non-limiting examples of types of glycosylation can include N-linked glycosylation, O-linked glycosylation, C-linked glycosylation, S-linked glycosylation, and glycation.

The terms “biological sample,” “biological specimen,” or “biospecimen” as used herein, generally refers to a specimen taken by sampling so as to be representative of the source of the specimen, typically, from a subject. A biological sample can be representative of an organism as a whole, specific tissue, cell type, or category or sub-category of interest. Biological samples may include, but are not limited to synovial fluid, whole blood, blood serum, blood plasma, urine, sputum, tissue, saliva, tears, spinal fluid, tissue section(s) obtained by biopsy; cell(s) that are placed in or adapted to tissue culture; sweat, mucous, fecal material, gastric fluid, abdominal fluid, amniotic fluid, cyst fluid, peritoneal fluid, pancreatic juice, breast milk, lung lavage, marrow, gastric acid, bile, semen, pus, aqueous humor, transudate, and the like including derivatives, portions and combinations of the foregoing. In some examples, biological samples include, but are not limited, to blood and/or plasma. In some examples, biological samples include, but are not limited, to urine or stool. Biological samples include, but are not limited, to saliva. Biological samples include, but are not limited, to tissue dissections and tissue biopsies. Biological samples include, but are not limited, any derivative or fraction of the aforementioned biological samples. The biological sample can include a macromolecule. The biological sample can include a small molecule. The biological sample can include a virus. The biological sample can include a cell or derivative of a cell. The biological sample can include an organelle. The biological sample can include a cell nucleus. The biological sample can include a rare cell from a population of cells. The biological sample can include any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological sample can include a constituent of a cell. The biological sample can include nucleotides (e.g., ssDNA, dsDNA, RNA), organelles, amino acids, peptides, proteins, carbohydrates, glycoproteins, or any combination thereof. The biological sample can include a matrix (e.g., a gel or polymer matrix) comprising a cell or one or more constituents from a cell (e.g., cell bead), such as DNA, RNA, organelles, proteins, or any combination thereof, from the cell. The biological sample may be obtained from a tissue of a subject. The biological sample can include a hardened cell. Such hardened cells may or may not include a cell wall or cell membrane. The biological sample can include one or more constituents of a cell but may not include other constituents of the cell. An example of such constituents may include a nucleus or an organelle. The biological sample may include a live cell. The live cell can be capable of being cultured.

The term “biomarker,” as used herein, generally refers to any measurable substance taken as a sample from a subject whose presence is indicative of some phenomenon. Non-limiting examples of such phenomenon can include a disease state, a condition, or exposure to a compound or environmental condition. In various embodiments described herein, biomarkers may be used for diagnostic purposes (e.g., to diagnose a health state, a disease state). The term “biomarker” can be used interchangeably with the term “marker.”

The term “denaturation,” as used herein, generally refers to any molecule that loses quaternary structure, tertiary structure, and secondary structure which is present in their native state. Non-limiting examples include proteins or nucleic acids being exposed to an external compound or environmental condition such as acid, base, temperature, pressure, radiation, etc.

The term “denatured protein,” as used herein, generally refers to a protein that loses quaternary structure, tertiary structure, and secondary structure which is present in their native state.

The terms “digestion” or “enzymatic digestion,” as used herein, generally refers to a biological process that employs enzymes to break specific amino acid peptide bonds. For example, digesting a peptide includes contacting the peptide with an digesting enzyme, e.g., trypsin to produce fragments of the glycopeptide. In some examples, a protease enzyme is used to digest a glycopeptide. The term “protease” refers to an enzyme that performs proteolysis or breakdown of large peptides into smaller polypeptides or individual amino acids. Examples of a protease include, but are not limited to, one or more of a serine protease, threonine protease, cysteine protease, aspartate protease, glutamic acid protease, metalloprotease, asparagine peptide lyase, and any combinations of the foregoing. Enzymatic digestion may be used in preparation for mass spectrometry using trypsin digestion protocols. Proteins may be digested using other proteases in preparation for mass spectrometry if access is limited to cleavage sites.

The term “disease state” as used herein, generally refers to a condition that affects the structure or function of an organism. Non-limiting examples of causes of disease states may include pathogens, immune system dysfunctions, cell damage caused by aging, cell damage caused by other factors (e.g., trauma and cancer). Disease states can include any state of a disease whether symptomatic or asymptomatic. Disease states can include disease stages of a disease progression. Disease states can cause minor, moderate, or severe disruptions in structure or function of an organism (e.g., a subject).

The term “fragment,” as used herein, generally refers to an ion fragmentation process which occurs in a MRM-MS instrument. Fragmenting may produce various fragments having the same mass but varying with respect to their charge, e.g., some biomarkers described herein produce more than one product m/z.

The terms “glycan” or “polysaccharide” as used herein, both generally refer to a carbohydrate residue of a glycoconjugate, such as the carbohydrate portion of a glycopeptide, glycoprotein, glycolipid, or proteoglycan. Glycans can include monosaccharides.

The term “glycopeptide” or “glycopolypeptide” as used herein, generally refers to a peptide or polypeptide comprising at least one glycan residue. In various embodiments, glycopeptides comprise carbohydrate moieties (e.g., one or more glycans) covalently attached to a side chain (i.e. R group) of an amino acid residue.

The term “glycopeptide fragment” or “glycosylated peptide fragment” or “glycopeptide” as used herein, generally refers to a glycosylated peptide (or glycopeptide) having an amino acid sequence that is the same as part (but not all) of the amino acid sequence of the glycosylated protein from which the glycosylated peptide is obtained, e.g., ion fragmentation within a MRM-MS instrument. MRM refers to multiple-reaction-monitoring. Unless specified otherwise, within the specification, “glycopeptide fragments” or “fragments of a glycopeptide” refer to the fragments produced directly by using a mass spectrometer optionally after the glycoprotein has been digested enzymatically to produce the glycopeptides.

The term “glycoprotein,” as used herein, generally refers to a protein having at least one glycan residue bonded thereto. In some examples, a glycoprotein is a protein with at least one oligosaccharide chain covalently bonded thereto. Examples of glycoproteins include but are not limited to the peptide structures including glycan molecules shown in the various Tables presented herein. A glycopeptide, as used herein, refers to a fragment of a glycoprotein, unless specified otherwise to the contrary.

The term “liquid chromatography,” as used herein, generally refers to a technique used to separate a sample into parts. Liquid chromatography can be used to separate, identify, and quantify components.

The term “mass spectrometry,” as used herein, generally refers to an analytical technique used to identify molecules. In various embodiments described herein, mass spectrometry can be involved in characterization and sequencing of proteins.

The term “m/z” or “mass-to-charge ratio,” as used herein, generally refers to an output value from a mass spectrometry instrument. In various embodiments, m/z can represent a relationship between the mass of a given ion and the number of elementary charges that it carries. The “m” in m/z stands for mass and the “z” stands for charge. In some embodiments, m/z can be displayed on an x-axis of a mass spectrum.

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “peptide,” as used herein, generally refers to amino acids linked by peptide bonds. Peptides can include amino acid chains between 10 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” As used herein, the phrase “peptide,” is meant to include glycopeptides unless stated otherwise.

The terms “protein” or “polypeptide” or “peptide” may be used interchangeably herein and generally refer to a molecule including at least three amino acid residues. Proteins can include polymer chains made of amino acid sequences linked together by peptide bonds. Proteins may be digested in preparation for mass spectrometry using trypsin digestion protocols. Proteins may be digested using other proteases in preparation for mass spectrometry if access is limited to cleavage sites.

The term “peptide structure,” as used herein, generally refers to peptides or a portion thereof or glycopeptides or a portion thereof. In various embodiments described herein, a peptide structure can include any molecule comprising at least two amino acids in sequence.

The term “reduction,” as used herein, generally refers to the gain of an electron by a substance. In various embodiments described herein, a sugar can directly bind to a protein, thereby, reducing the amino acid to which it binds. Such reducing reactions can occur in glycosylation. In various embodiments, reduction may be used to break disulfide bonds between two cysteines.

The term “sample,” as used herein, generally refers to a sample from a subject of interest and may include a biological sample of a subject. The sample may include a cell sample. The sample may include a cell line or cell culture sample. The sample can include one or more cells. The sample can include one or more microbes. The sample may include a nucleic acid sample or protein sample. The sample may also include a carbohydrate sample or a lipid sample. The sample may be derived from another sample. The sample may include a tissue sample, such as a biopsy, core biopsy, needle aspirate, or fine needle aspirate. The sample may include a fluid sample, such as a blood sample, urine sample, or saliva sample. The sample may include a skin sample. The sample may include a cheek swab. The sample may include a plasma or serum sample. The sample may include a cell-free or cell free sample. A cell-free sample may include extracellular polynucleotides. The sample may originate from blood, plasma, serum, urine, saliva, mucosal excretions, sputum, stool, or tears. The sample may originate from red blood cells or white blood cells. The sample may originate from feces, spinal fluid, CNS fluid, gastric fluid, amniotic fluid, cyst fluid, peritoneal fluid, marrow, bile, other body fluids, tissue obtained from a biopsy, skin, or hair.

The term “sequence,” as used herein, generally refers to a biological sequence including one-dimensional monomers that can be assembled to generate a polymer. Non-limiting examples of sequences include nucleotide sequences (e.g., ssDNA, dsDNA, and RNA), amino acid sequences (e.g., proteins, peptides, and polypeptides), and carbohydrates (e.g., compounds including Cₘ (H₂O)ₙ).

The term “subject,” as used herein, generally refers to an animal, such as a mammal (e.g., human) or avian (e.g., bird), or other organism, such as a plant. For example, the subject can include a vertebrate, a mammal, a rodent (e.g., a mouse), a primate, a simian or a human. Animals may include, but are not limited to, farm animals, sport animals, and pets. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. A subject can include a microorganism or microbe (e.g., bacteria, fungi, archaea, viruses). However, in the context of diagnosing ovarian cancer, the subject is female unless explicitly specified otherwise. A subject may be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

As used herein, a “target glycopeptide analyte,” may refer to a peptide structure (e.g., glycosylated or aglycosylated/non-glycosylated), a fraction of a peptide structure, a sub-structure (e.g., a glycan or a glycosylation site) of a peptide structure, a product of one or more of the above listed structures and sub-structures, associated detection molecules (e.g., signal molecule, label, or tag), or an amino acid sequence that can be measured by mass spectrometry.

As used herein, a “peptide data set,” may be used interchangeably with “peptide structure data” and can refer to any data of or relating to a peptide from a resulting mass spectrometry run. A peptide data set can comprise data obtained from a sample or biological sample using mass spectrometry. A peptide dataset can comprise data relating to an external standard, data relating to an internal standard, and data relating to a target glycopeptide analyte of a sample. A peptide data set can result from analysis originating from a single run. In some embodiments, the peptide data set can include raw abundance and mass to charge ratios for one or more peptides.

As used herein, a “a transition,” may refer to or identify a peptide structure. In some embodiments, a transition can refer to the specific pair of m/z values associated with a precursor ion and a product or fragment ion.

As used herein, a “non-glycosylated endogenous peptide” (“NGEP”) may refer to a peptide structure that does not comprise a glycan molecule. In various embodiments, an NGEP and a target glycopeptide analyte can originate from the same subject. In various embodiments, an NGEP and a target glycopeptide analyte may be derived from the same protein sequence. In some embodiments, the NGEP and the target glycopeptide analyte may be derived from or include the same peptide sequence. In various embodiments, an NGEP can be labeled with an isotope in preparation for mass spectrometry analysis.

As used herein, “abundance,” may refer to a quantitative value generated using mass spectrometry. In various embodiments, the quantitative value may relate to the amount of a particular peptide structure. In some embodiments, the quantitative value may comprise an amount of an ion produced using mass spectrometry. In some embodiments, the quantitative value may be expressed as an m/z value. In other embodiments, the quantitative value may be expressed in atomic mass units.

As used herein, “relative abundance,” may refer to a comparison of two or more abundances. In various embodiments, the comparison may comprise comparing one peptide structure to a total number of peptide structures. In some embodiments, the comparison may comprise comparing one peptide glycoform (e.g., two identical peptides differing by one or more glycans) to a set of peptide glycoforms. In some embodiments, the comparison may comprise comparing a number of ions having a particular m/z ratio by a total number of ions detected. In various embodiments, a relative abundance can be expressed as a ratio. In other embodiments, a relative abundance can be expressed as a percentage. Relative abundance can be presented on a y-axis of a mass spectrum plot.

As used herein, an “internal standard,” may refer to something that can be contained (e.g., spiked-in) in the same sample as a target glycopeptide analyte undergoing mass spectrometry analysis. Internal standards can be used for calibration purposes. Additionally, internal standards can be used in the systems and method described herein. In some aspects, an internal standard can be selected based on similarity m/z and or retention times and can be a “surrogate” if a specific standard is too costly or unavailable. Internal standards can be heavy labeled or non-heavy labeled.

### III. Overview of Exemplary Workflow

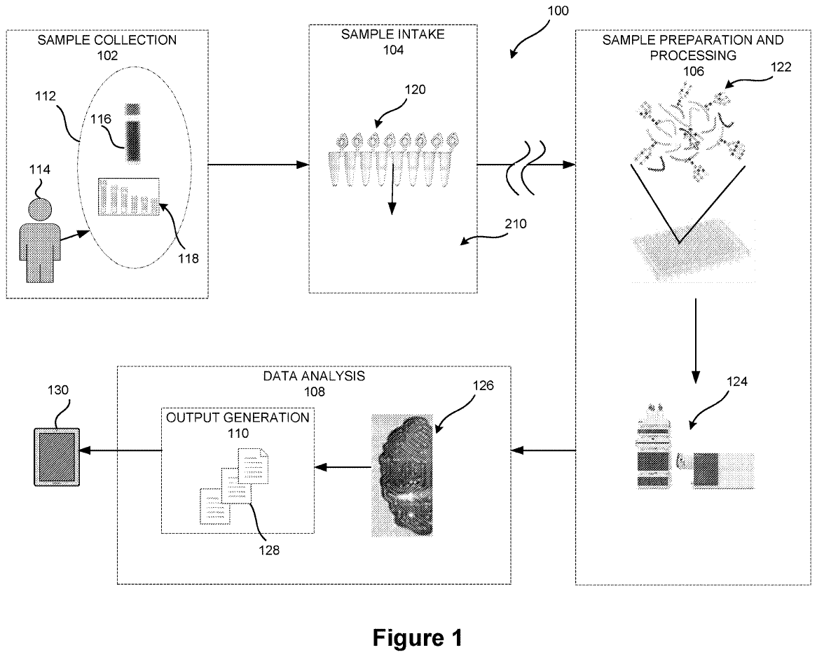

FIG. 1 is a schematic diagram of an exemplary workflow 100 for the detection of peptide structures associated with a disease state for use in diagnosis and/or treatment in accordance with one or more embodiments. Workflow 100 may include various operations including, for example, sample collection 102, sample intake 104, sample preparation and processing 106, data analysis 108, and output generation 110.

Sample collection 102 may include, for example, obtaining a biological sample 112 of one or more subjects, such as subject 114. Biological sample 112 may take the form of a specimen obtained via one or more sampling methods. Biological sample 112 may be representative of subject 114 as a whole or of a specific tissue, cell type, or other category or sub-category of interest. Biological sample 112 may be obtained in any of a number of different ways. In various embodiments, biological sample 112 includes whole blood sample 116 obtained via a blood draw. In other embodiments, biological sample 112 includes set of aliquoted samples 118 that includes, for example, a serum sample, a plasma sample, a blood cell (e.g., white blood cell (WBC), red blood cell (RBC) sample, another type of sample, or a combination thereof. Biological samples 112 may include nucleotides (e.g., ssDNA, dsDNA, RNA), organelles, amino acids, peptides, proteins, carbohydrates, glycoproteins, or any combination thereof.

In various embodiments, a single run can analyze a sample (e.g., the sample including a peptide analyte), an external standard (e.g., an NGEP of a serum sample), and an internal standard. As such, abundance or raw abundance for the external standard, the internal standard, and target glycopeptide analyte can be determined by mass spectrometry in the same run.

In various embodiments, external standards may be analyzed prior to analyzing samples. In various embodiments, the external standards can be run independently between the samples. In some embodiments, external standards can be analyzed after every 1, 2, 3, 4, 5, 6, 7, 8, 9, 10, 11, 12, 13, 14, 15, 16, 17, 18, 19, 20, or more experiments. In various embodiments, external standard data can be used in some or all of the normalization systems and methods described herein. In additional embodiments, blank samples may be processed to prevent column fouling.

Sample intake 104 may include one or more various operations such as, for example, aliquoting, registering, processing, storing, thawing, and/or other types of operations. In one or more embodiments, when biological sample 112 includes whole blood sample 116, sample intake 104 includes aliquoting whole blood sample 116 to form a set of aliquoted samples that can then be sub-aliquoted to form set of samples 120.

Sample preparation and processing 106 may include, for example, one or more operations to form set of peptide structures 122. In various embodiments, set of peptide structures 122 may include various fragments of unfolded proteins that have undergone digestion and may be ready for analysis.

Further, sample preparation and processing 106 may include, for example, data acquisition 124 based on set of peptide structures 122. For example, data acquisition 124 may include use of, for example, but is not limited to, a liquid chromatography/mass spectrometry (LC/MS) system.

Data analysis 108 may include, for example, peptide structure analysis 126. In some embodiments, data analysis 108 also includes output generation 110. In other embodiments, output generation 110 may be considered a separate operation from data analysis 108. Output generation 110 may include, for example, generating final output 128 based on the results of peptide structure analysis 126. Final output 128 may be used for determining research, diagnosis, and/or treatment.

In various embodiments, final output 128 is comprised of one or more outputs. Final output 128 may take various forms. For example, final output 128 may be a report that includes, for example, a diagnosis output, a treatment output (e.g., a treatment design output, a treatment plan output, or combination thereof), analyzed data (e.g., relativized and normalized) or combination thereof. In some embodiments, report can comprise a target glycopeptide analyte concentration as a function of the NGEP concentration value and the normalized abundance. In some embodiments, final output 128 may be an alert (e.g., a visual alert, an audible alert, etc.), a notification (e.g., a visual notification, an audible notification, an email notification, etc.), an email output, or a combination thereof. In some embodiments, final output 128 may be sent to remote system 130 for processing. Remote system 130 may include, for example, a computer system, a server, a processor, a cloud computing platform, cloud storage, a laptop, a tablet, a smartphone, some other type of mobile computing device, or a combination thereof.

In other embodiments, workflow 100 may optionally exclude one or more of the operations described herein and/or may optionally include one or more other steps or operations other than those described herein (e.g., in addition to and/or instead of those described herein). Accordingly, workflow 100 may be implemented in any of a number of different ways for use in the research, diagnosis, and/or treatment of a disease state.

### IV. Detection and Quantification of Peptide Structures

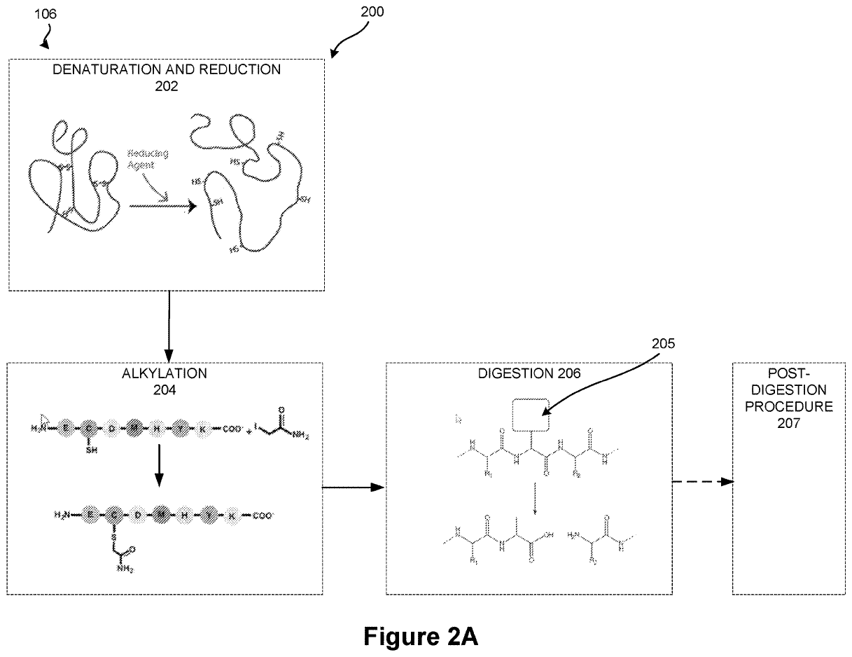

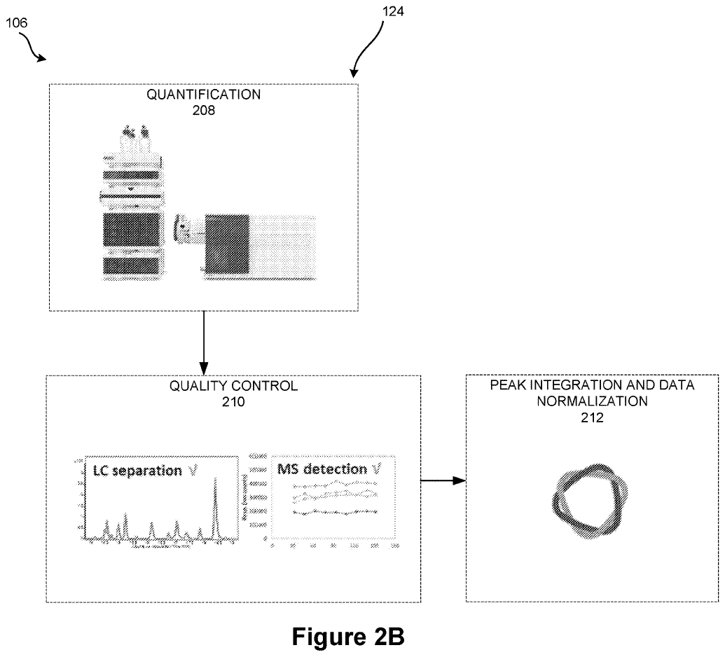

FIGS. 2A and 2B are schematic diagrams of a workflow for sample preparation and processing 106 in accordance with one or more embodiments. FIGS. 2A and 2B are described with continuing reference to FIG. 1. Sample preparation and processing 106 may include, for example, preparation workflow 200 shown in FIG. 2A and data acquisition 124 shown in FIG. 2B.

### IV.A. Sample Preparation and Processing

FIG. 2A is a schematic diagram of preparation workflow 200 in accordance with one or more embodiments. Preparation workflow 200 may be used to prepare a sample, such as a sample of set of samples 120 in FIG. 1, for analysis via data acquisition 124. For example, this analysis may be performed via mass spectrometry (e.g., LC-MS). In various embodiments, preparation workflow 200 may include denaturation and reduction 202, alkylation 204, and digestion 206. All areas of the preparation workflow can cause inconsistency between different samples and different experiments, necessitating, the improved normalization systems and methods described herein and throughout.

In general, polymers, such as proteins, in their native form, can fold to include secondary, tertiary, and/or other higher order structures. Such higher order structures may functionalize proteins to complete tasks (e.g., enable enzymatic activity) in a subject. Further, such higher order structures of polymers may be maintained via various interactions between side chains of amino acids within the polymers. Such interactions can include ionic bonding, hydrophobic interactions, hydrogen bonding, and disulfide linkages between cysteine residues. However, when using analytic systems and methods, including mass spectrometry, unfolding such polymers (e.g., peptide/protein molecules) may be desired to obtain sequence information. In some embodiments, unfolding a polymer may include denaturing the polymer, which may include, for example, linearizing the polymer.

In one or more embodiments, denaturation and reduction 202 can be used to disrupt higher order structures (e.g., secondary, tertiary, quaternary, etc.) of one or more proteins (e.g., polypeptides and peptides) in a sample (e.g., one of set of samples 120 in FIG. 1). Denaturation and reduction 202 includes, for example, a denaturation procedure and a reduction procedure. In some embodiments, the denaturation procedure may be performed using, for example, thermal denaturation, where heat is used as a denaturing agent. The thermal denaturation can disrupt ionic bonding, hydrophobic interactions, and/or hydrogen bonding.

In various embodiments, the denaturation procedure may include using one or more denaturing agents. In one or more embodiments, the denaturation procedure may include using temperature. In one or more embodiments, the denaturation procedure may include using one or more denaturing agents in combination with heat. These one or more denaturing agents may include, for example, but are not limited to, any number of chaotropic salts (e.g., urea, guanidine), surfactants (e.g., sodium dodecyl sulfate (SDS), beta octyl glucoside, Triton X-100), or combination thereof. In some cases, such denaturing agents may be used in combination with heat when sample preparation workflow further includes a cleanup procedure.

The resulting one or more denatured (e.g., unfolded, linearized) proteins may then undergo further processing in preparation of analysis. For example, a reduction procedure may be performed in which one or more reducing agents are applied. In various embodiments, a reducing agent can produce an alkaline pH. A reducing agent may take the form of, for example, without limitation, dithiothreitol (DTT), tris(2-carboxyethyl)phosphine (TCEP), or some other reducing agent. The reducing agent may reduce (e.g., cleave) the disulfide linkages between cysteine residues of the one or more denatured proteins to form one or more reduced proteins.

In various embodiments, the one or more reduced proteins resulting from denaturation and reduction 202 may undergo a process to prevent the reformation of disulfide linkages between, for example, the cysteine residues of the one or more reduced proteins. This process may be implemented using alkylation 204 to form one or more alkylated proteins. For example, alkylation 204 may be used to add an acetamide group to a sulfur on each cysteine residue to prevent disulfide linkages from reforming. In various embodiments, an acetamide group can be added by reacting one or more alkylating agents with a reduced protein. The one or more alkylating agents may include, for example, one or more acetamide salts. An alkylating agent may take the form of, for example, iodoacetamide (IAA), 2-chloroacetamide, some other type of acetamide salt, or some other type of alkylating agent.

In some embodiments, alkylation 204 may include a quenching procedure. The quenching procedure may be performed using one or more reducing agents (e.g., one or more of the reducing agents described above).

In various embodiments, the one or more alkylated proteins formed via alkylation 204 can then undergo digestion 206 in preparation for analysis (e.g., mass spectrometry analysis). Digestion 206 of a protein may include cleaving the protein at or around one or more cleavage sites (e.g., site 205 which may be one or more amino acid residues). For example, without limitation, an alkylated protein may be cleaved at the carboxyl side of the lysine or arginine residues. This type of cleavage may break the protein into various segments, which include one or more peptide structures (e.g., glycosylated or aglycosylated).

In various embodiments, digestion 206 is performed using one or more proteolysis catalysts. For example, an enzyme can be used in digestion 206. In some embodiments, the enzyme takes the form of trypsin. In other embodiments, one or more other types of enzymes (e.g., proteases) may be used in addition to or in place of trypsin. These one or more other enzymes include, but are not limited to, LysC, LysN, AspN, GluC, and ArgC. In some embodiments, digestion 206 may be performed using tosyl phenylalanyl chloromethyl ketone (TPCK)-treated trypsin, one or more engineered forms of trypsin, one or more other formulations of trypsin, or a combination thereof. In some embodiments, digestion 206 may be performed in multiple steps, with each involving the use of one or more digestion agents. For example, a secondary digestion, tertiary digestion, etc. may be performed. In one or more embodiments, trypsin is used to digest serum samples. In one or more embodiments, trypsin/LysC cocktails are used to digest plasma samples.

In some embodiments, digestion 206 further includes a quenching procedure. The quenching procedure may be performed by acidifying the sample (e.g., to a pH<3). In some embodiments, formic acid may be used to perform this acidification.

In various embodiments, preparation workflow 200 further includes post-digestion procedure 207. Post-digestion procedure 207 may include, for example, a cleanup procedure. The cleanup procedure may include, for example, the removal of unwanted components in the sample that results from digestion 206. For example, unwanted components may include, but are not limited to, inorganic ions, surfactants, etc. In some embodiments, post-digestion procedure 207 further includes a procedure for the addition of heavy-labeled peptide internal standards.

Although preparation workflow 200 has been described with respect to a sample created or taken from biological sample 112 that is blood-based (e.g., a whole blood sample, a plasma sample, a serum sample, etc.), sample preparation workflow 200 may be similarly implemented for other types of samples (e.g., tears, urine, tissue, interstitial fluids, sputum, etc.) to produce set of peptides structures 122.

### IV.B. Peptide Structure Identification and Quantitation

FIG. 2B is a schematic diagram of data acquisition 124 in accordance with one or more embodiments. In various embodiments, data acquisition 124 can commence following sample preparation 200 described in FIG. 2A. In various embodiments, data acquisition 124 can comprise quantification 208, quality control 210, and peak integration and normalization 212.

In various embodiments, targeted quantification 208 of peptides and glycopeptides can incorporate use of liquid chromatography-mass spectrometry LC/MS instrumentation. For example, LC-MS/MS, or tandem MS may be used. In general, LC/MS (e.g., LC-MS/MS) can combine the physical separation capabilities of liquid chromatograph (LC) with the mass analysis capabilities of mass spectrometry (MS). According to some embodiments described herein, this technique allows for the separation of digested peptides to be fed from the LC column into the MS ion source through an interface.

In various embodiments, any LC/MS device can be incorporated into the workflow described herein. In various embodiments, an instrument or instrument system suited for identification and targeted quantification 208 may include, for example, a Triple Quadrupole LC/MS™. In various embodiments, targeted quantification 208 is performed using multiple reaction monitoring mass spectrometry (MRM-MS).

In various embodiments described herein, identification of a particular protein or peptide and an associated quantity can be assessed. In various embodiments described herein, identification of a particular glycan and an associated quantity can be assessed. In various embodiments described herein, particular glycans can be matched to a glycosylation site on a protein or peptide and the abundances measured.

In some cases, targeted quantification 208 includes using a specific collision energy associated for the appropriate fragmentation to consistently see an abundant product ion. Glycopeptide structures may have a lower collision energy than aglycosylated peptide structures. When analyzing a sample that includes glycopeptide structures, the source voltage and gas temperature may be lowered as compared to generic proteomic analysis.

In various embodiments, quality control 210 procedures can be put in place to optimize data quality. In various embodiments, measures can be put in place allowing only errors within acceptable ranges outside of an expected value. In various embodiments, employing statistical models (e.g., using Westgard rules) can assist in quality control 210. For example, quality control 210 may include, for example, assessing the retention time and abundance of representative peptide structures (e.g., glycosylated and/or aglycosylated) and spiked-in internal standards, in either every sample, or in each quality control sample (e.g., pooled serum digest).

Peak integration and normalization 212 may be performed to process the data that has been generated and transform the data into a format for analysis. For example, peak integration and normalization 212 may include converting abundance data for various product ions that were detected for a selected peptide structure into a single quantification metric (e.g., a relative quantity, an adjusted quantity, a normalized quantity, a relative concentration, an adjusted concentration, a normalized concentration, etc.) for that peptide structure. In some embodiments, peak integration and normalization 212 may be performed using one or more of the techniques described in U.S. Patent Publication No. 2020/0372973A1 and/or US Patent Publication No. 2020/0240996A1, the disclosures of which are incorporated by reference herein in their entireties.

### V. Peptide Structure Data Analysis

### V.A. Exemplary System for Peptide Structure Data Analysis

### V.A.1. Analysis System for Peptide Structure Data Analysis

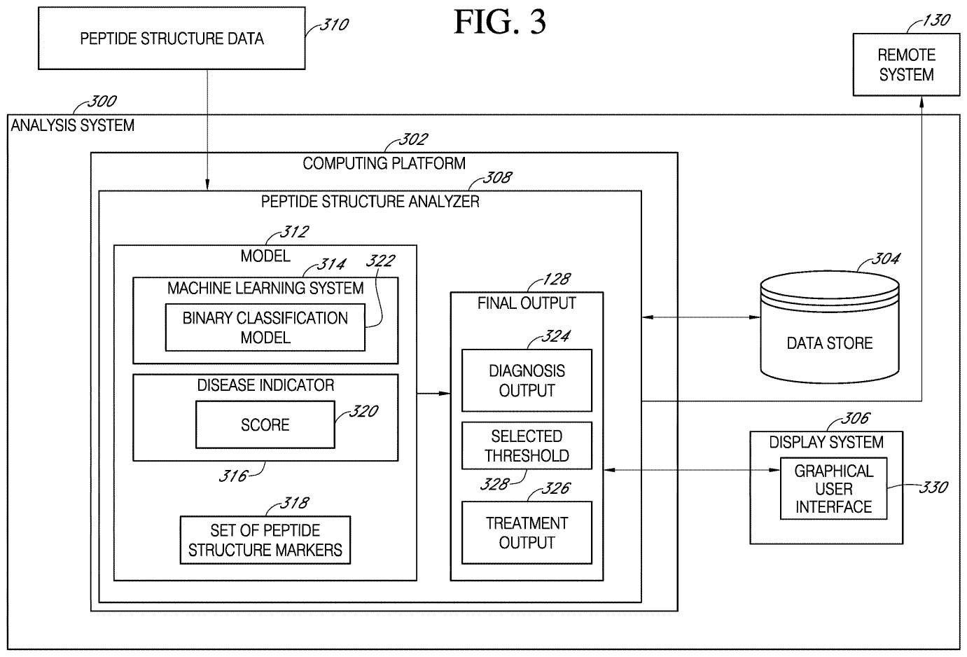

FIG. 3 is a block diagram of an analysis system 300 in accordance with one or more embodiments. Analysis system 300 can be used to both detect and analyze various peptide structures that have been associated to various disease states. Analysis system 300 is one example of an implementation for a system that may be used to perform data analysis 108 in FIG. 1. Thus, analysis system 300 is described with continuing reference to workflow 100 as described in FIGS. 1, 2A, and/or 2B.

Analysis system 300 may include computing platform 302 and data store 304. In some embodiments, analysis system 300 also includes display system 306. Computing platform 302 may take various forms. In one or more embodiments, computing platform 302 includes a single computer (or computer system) or multiple computers in communication with each other. In other examples, computing platform 302 takes the form of a cloud computing platform.

Data store 304 and display system 306 may each be in communication with computing platform 302. In some examples, data store 304, display system 306, or both may be considered part of or otherwise integrated with computing platform 302. Thus, in some examples, computing platform 302, data store 304, and display system 306 may be separate components in communication with each other, but in other examples, some combination of these components may be integrated together. Communication between these different components may be implemented using any number of wired communications links, wireless communications links, optical communications links, or a combination thereof.

Analysis system 300 includes, for example, peptide structure analyzer 308, which may be implemented using hardware, software, firmware, or a combination thereof. In one or more embodiments, peptide structure analyzer 308 is implemented using computing platform 302.

Peptide structure analyzer 308 receives peptide structure data 310 for processing. Peptide structure data 310 may be, for example, the peptide structure data that is output from sample preparation and processing 106 in FIGS. 1, 2A, and 2B. Accordingly, peptide structure data 310 may correspond to set of peptide structures 122 identified for biological sample 112 and may thereby correspond to biological sample 112.

Peptide structure data 310 can be sent as input into peptide structure analyzer 308, retrieved from data store 304 or some other type of storage (e.g., cloud storage), accessed from cloud storage, or obtained in some other manner. In some cases, peptide structure data 310 may be retrieved from data store 304 in response to (e.g., directly or indirectly based on) receiving user input entered by a user via an input device.

Peptide structure analyzer 308 includes model 312 that is configured to receive peptide structure data 310 for processing. Model 312 may be implemented in any of a number of different ways. Model 312 may be implemented using any number of models, functions, equations, algorithms, and/or other mathematical techniques.

In one or more embodiments, model 312 includes machine learning system 314, which may itself be comprised of any number of machine learning models and/or algorithms. For example, machine learning system 314 may include, but is not limited to, at least one of a deep learning model, a neural network, a linear discriminant analysis model, a quadratic discriminant analysis model, a support vector machine, a random forest algorithm, a nearest neighbor algorithm (e.g., a k-Nearest Neighbors algorithm), a combined discriminant analysis model, a k-means clustering algorithm, an unsupervised model, a multivariable regression model, a penalized multivariable regression model, or another type of model. In various embodiments, model 312 includes a machine learning system 314 that comprises any number of or combination of the models or algorithms described above.

In various embodiments, model 312 analyzes peptide structure data 310 to generate disease indicator 316 that indicates whether the biological sample is positive for an ovarian cancer disease state based on set of peptide structures 318 identified as being associated with the ovarian cancer disease state. Peptide structure data 310 may include quantification data for the plurality of peptide structures. Quantification data for a peptide structures can include at least one of an abundance, a relative abundance, a normalized abundance, a relative quantity, an adjusted quantity, a normalized quantity, a relative concentration, an adjusted concentration, or a normalized concentration. For example, peptide structure data 310 may include a set of quantification metrics for each peptide structure of a plurality of peptide structures. A quantification metric for a peptide structure may be selected as one of a relative quantity, an adjusted quantity, a normalized quantity, a relative abundance, an adjusted abundance, and a normalized abundance. In some cases, a quantification metric for a peptide structure is selected from one of a relative concentration, an adjusted concentration, and a normalized concentration. In one or more embodiments, the quantification metrics used are normalized abundances. In this manner, peptide structure data 310 may provide abundance information about the plurality of peptide structures with respect to biological sample 112.

Disease indicator 316 may take various forms. In some examples, disease indicator 316 includes a classification that indicates whether or not the subject is positive for the ovarian cancer disease state. In various embodiments, disease indicator 316 can include a score 320. Score 320 indicates whether the ovarian cancer disease state is present or not. For example, score 320 may be, a probability score that indicates how likely it is that the biological sample 112 evidences the presence of the ovarian cancer disease state.

In one or more embodiments, a peptide structure of set of peptide structures 318 comprises a glycosylated peptide structure, or glycopeptide structure, that is defined by a peptide sequence and a glycan structure attached to a linking site of the peptide sequence quantity. For example, the peptide structure may be a glycopeptide or a portion of a glycopeptide. In some embodiments, a peptide structure of set of peptide structures 318 comprises an aglycosylated peptide structure that is defined by a peptide sequence. For example, the peptide structure may be a peptide or a portion of a peptide and may be referred to as a quantification peptide.

Set of peptide structures 318 may be identified as being those most predictive or relevant to the ovarian cancer disease state based on training of model 312. In one or more embodiments, set of peptide structures 318 includes at least one, at least two, or at least three peptide structures from a first group of peptide structures (peptide structures PS-1 through PS-10) identified in Table 1 in Section VI.A. or at least one, at least two, or at least three peptide structures from a second group of peptide structures (peptide structures PS-5 and PS-11 through PS-34) identified in Table 2 in Section VI.A. For example, in one or more embodiments, set of peptide structures 318 includes at least 1, at least 2, at least 3, at least 4, at least 5, at least 6, at least 7, at least 8, at least 9, or all 10 of the peptide structures identified in Table 1 below in Section VI.A. In one or more other embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures identified in Table 2 below in Section VI.A. In one or more embodiments, set of peptide structures 318 includes at least peptide structure PS-5, which is identified in both Table 1 and Table 2. In some cases, the number of peptide structures selected from Table 1 for inclusion in set of peptide structures 318 may be based on, for example, a desired level of accuracy.

In one or more embodiments, set of peptide structures 318 includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures identified in Table 3 below in Section VI.A. In one or more embodiments, set of peptide structures 318 includes at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, or all 61 of the peptide structures listed in Tables 1, 2, and 3.

In various embodiments, machine learning system 314 takes the form of binary classification model 322. Binary classification model 322 may include, for example, but is not limited to, a regression model. Binary classification model 322 may include, for example, a penalized multivariable regression model that is trained to identify set of peptide structures 318 from a plurality of (or panel of) peptide structures identified in various subjects. Binary classification model 322 may be trained to identify weight coefficients for peptide structures and those peptide structures having non-zero weights or weight coefficients above a selected threshold (e.g., absolute weight coefficient above 0.0, 0.01, 0.05, 0.1, 0.015, 0.2, etc.) may be selected for inclusion in set of peptide structures 318.

Peptide structure analyzer 308 may generate final output 128 based on disease indicator 316 output by model 312. In other embodiments, final output 128 may be an output generated by model 312.

In some embodiments, final output 128 includes disease indicator 316. In one or more embodiments, final output 128 includes diagnosis output 324, treatment output 326, or both. Diagnosis output 324 may include, for example, a diagnosis for the ovarian cancer disease state. The diagnosis can include a positive diagnosis or a negative diagnosis for the ovarian cancer disease state. In one or more embodiments, generating diagnosis output 324 may include comparing score 320 to selected threshold 328 to determine the diagnosis. Selected threshold 328 may be, for example, without limitation, a score between 0.30 and 0.65 (e.g., 0.4, 0.5, 0.6, etc.). For example, when selected threshold 328 is set to 0.5, a score 320 above 0.5 (or at or above 0.5) may indicate the presence of the ovarian cancer disease state and be output in diagnosis output 324 as a positive diagnosis. A score 320 below 0.5 (or at or below 0.5) may indicate that the ovarian cancer disease state is not present and be output in diagnosis output 324 as a negative diagnosis. In one or more embodiments, a negative diagnosis indicates that the subject is healthy. In one or more embodiments, a negative diagnosis indicates that a detected pelvic tumor (or mass) is benign.

In one or more embodiments, when disease indicator 316 and/or diagnosis output 324 indicate a positive diagnosis for the ovarian cancer disease state, a biopsy may be recommended. For example, a biopsy of the subject may be performed in response to disease indicator 316 and/or diagnosis output 324 indicating a positive diagnosis for the ovarian cancer disease state. In some embodiments, peptide structure analyzer 308 (or another system implemented on computing platform 302) may generate a report recommending that a biopsy is to be performed for the subject in response to disease indicator 316 and/or diagnosis output 324 indicating a positive diagnosis for the ovarian cancer disease state. In other embodiments, peptide structure analyzer 308 may send diagnosis final output 128 to remote system 130 over one or more wireless, wired, and/or optical communications links and remote system 130 may generate a report recommending that a biopsy is to be performed for the subject in response to disease indicator 316 and/or diagnosis output 324 indicating a positive diagnosis for the ovarian cancer disease state. The biopsy may be used to confirm the diagnosis to determine whether or not to administer treatment and/or how quickly to administer treatment. When disease indicator 316 and/or diagnosis output 324 indicate a negative diagnosis for the ovarian cancer disease state (e.g., benign pelvic tumor), the report that is generated by peptide structure analyzer 308, remote system 130, or some other system implemented on computing platform 142 may recommend a period of monitoring for the subject. For example, a negative diagnosis indication by disease indicator 316 and/or diagnosis output 324 may thus help prevent unnecessary treatment or overtreatment of the subject.

Treatment output 326 may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Final output 128 may be sent to remote system 130 for processing in some examples. In other embodiments, final output 128 may be displayed on graphical user interface 330 in display system 306 for viewing by a human operator.

### V.A.2. Computer Implemented System

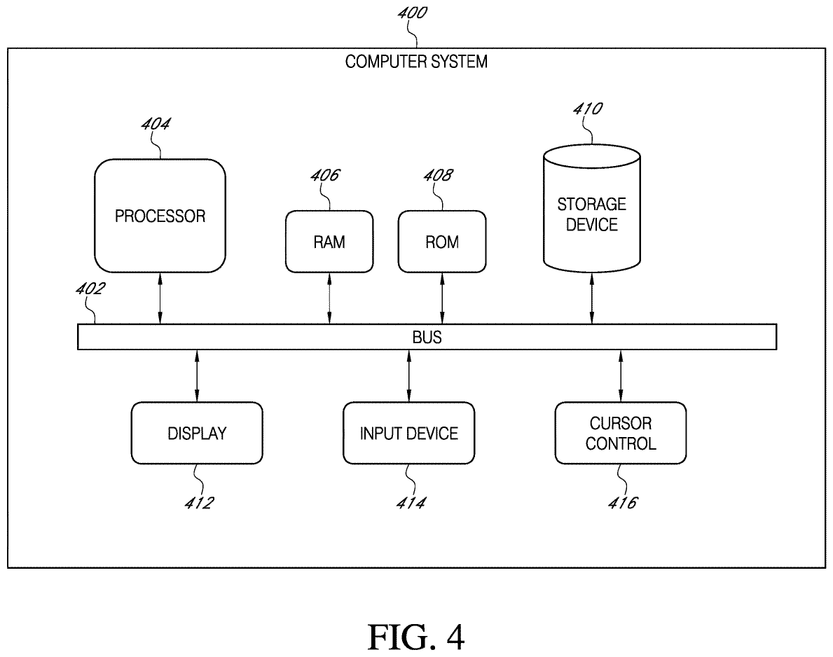

FIG. 4 is a block diagram of a computer system in accordance with various embodiments. Computer system 400 may be an example of one implementation for computing platform 302 described above in FIG. 3.

In one or more examples, computer system 400 can include a bus 402 or other communication mechanism for communicating information, and a processor 404 coupled with bus 402 for processing information. In various embodiments, computer system 400 can also include a memory, which can be a random-access memory (RAM) 406 or other dynamic storage device, coupled to bus 402 for determining instructions to be executed by processor 404. Memory also can be used for storing temporary variables or other intermediate information during execution of instructions to be executed by processor 404. In various embodiments, computer system 400 can further include a read only memory (ROM) 408 or other static storage device coupled to bus 402 for storing static information and instructions for processor 404. A storage device 410, such as a magnetic disk or optical disk, can be provided and coupled to bus 402 for storing information and instructions.

In various embodiments, computer system 400 can be coupled via bus 402 to a display 412, such as a cathode ray tube (CRT), liquid crystal display (LCD), or light emitting diode (LED) for displaying information to a computer user. An input device 414, including alphanumeric and other keys, can be coupled to bus 402 for communicating information and command selections to processor 404. Another type of user input device is a cursor control 416, such as a mouse, a joystick, a trackball, a gesture input device, a gaze-based input device, or cursor direction keys for communicating direction information and command selections to processor 404 and for controlling cursor movement on display 412. This input device 414 typically has two degrees of freedom in two axes, a first axis (e.g., x) and a second axis (e.g., y), that allows the device to specify positions in a plane. However, it should be understood that input devices 414 allowing for three-dimensional (e.g., x, y, and z) cursor movement are also contemplated herein.

Consistent with certain implementations of the present teachings, results can be provided by computer system 400 in response to processor 404 executing one or more sequences of one or more instructions contained in RAM 406. Such instructions can be read into RAM 406 from another computer-readable medium or computer-readable storage medium, such as storage device 410. Execution of the sequences of instructions contained in RAM 406 can cause processor 404 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 404 for execution. Such a medium can take many forms, including but not limited to, non-volatile media, volatile media, and transmission media. Examples of non-volatile media can include, but are not limited to, optical, solid state, magnetic disks, such as storage device 410. Examples of volatile media can include, but are not limited to, dynamic memory, such as RAM 406. Examples of transmission media can include, but are not limited to, coaxial cables, copper wire, and fiber optics, including the wires that comprise bus 402.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 404 of computer system 400 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 400 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 400, whereby processor 404 would execute the analyses and determinations provided by these engines, subject to instructions provided by any one of, or a combination of, the memory components RAM 406, ROM, 408, or storage device 410 and user input provided via input device 414.

### VI. Exemplary Methodologies Relating to Diagnosis Based on Peptide Structure Data Analysis

### VI.A. Exemplary Methodology-Based on Tables 1 and 2

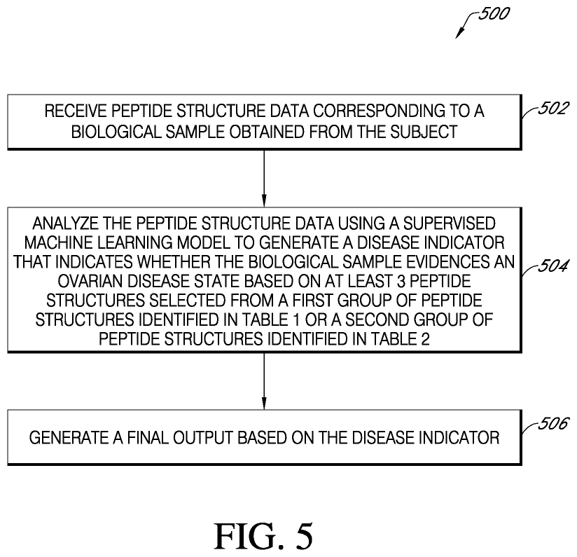

FIG. 5 is a flowchart of a process for diagnosing a subject with respect to an ovarian cancer disease state in accordance with one or more embodiments. Process 500 may be implemented using, for example, at least a portion of workflow 100 as described in FIGS. 1, 2A, and 2B and/or analysis system 300 as described in FIG. 3. Process 500 may be used to generate a final output that includes at least a diagnosis output for the subject.

Step 502 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 1 or Table 2, with the peptide sequence being one of SEQ ID NOS: 11-19 in Table 1 or one of SEQ ID NOS: 14, 15, and 31-46 in Table 2, the SEQ ID NOS being defined in Table 5 below.

Step 504 includes analyzing the peptide structure data using a supervised machine learning model to generate a disease indicator that indicates whether the biological sample evidences an ovarian cancer disease state based on at least three peptide structures selected from a first group of peptide structures identified in Table 1 (below) or a second group of peptide structures identified in Table 2 (below). In step 504, the first and second groups of peptide structures are associated with the ovarian cancer disease state. The first group of peptide structures is listed in Table 1 with respect to relative significance to the disease indicator. The second group of peptide structures is listed in Table 2 with respect to relative significance to the disease indicator.

The first group of peptide structures in Table 1 includes peptide structures that have been determined relevant to distinguishing at least between ovarian cancer (e.g., EOC) and a healthy state. For example, the first group of peptide structures may be used to predict the probability of EOC for use in clinically screening patients. In one or more embodiments, the first group of peptide structures in Table 1 may also be peptide structures that have been determined relevant to distinguishing between ovarian cancer (e.g., EOC) and a benign tumor state (e.g., a benign pelvic tumor). For example, the first group of peptide structures may be used to clinically triage patients that have been identified as having pelvic tumors to determine the probability that such a tumor evidences EOC.

The second group of peptide structures in Table 2 includes peptide structures that have been determined relevant to distinguishing at least between ovarian cancer (e.g., EOC) and the benign tumor state (e.g., a benign pelvic tumor). For example, the second group of peptide structures may be used to clinically triage patients that have been identified as having pelvic tumors to determine the probability that such a tumor evidences EOC. In this manner, the second group of peptide structures may predict malignancy of an identified pelvic tumor. In one or more embodiments, the at least 3 peptide structures includes at least 3, at least 4, at least 5, at least 6, at least 7, at least 8, at least 9, or all 10 of the peptide structures PS-1 to PS-10 in Table 1. In some embodiments, the at least 3 peptide structures include at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-5 and PS-11 through PS-34 in Table 1. In some embodiments, the at least 3 peptide structures includes at least PS-5, which is present in both Table 1 and Table 2.

In one or more embodiments, step 504 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 504 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

The peptide structure profile for a given peptide structure may include a corresponding feature—relative abundance, concentration, site occupancy—for that peptide structure. The relative abundance may be a normalized relative abundance; the concentration may be normalized concentration. In some cases, two peptide structure profiles may be computed for the same peptide structure, each profile corresponding to a different feature. For example, a first peptide structure profile may include a relative abundance for a corresponding peptide structure and a second peptide structure profile may include a concentration for the same corresponding peptide structure.

In various embodiments, the disease indicator comprises a probability that the biological sample is positive for the ovarian cancer disease state and the supervised machine learning model is configured to generate an output that identifies the biological sample as either evidencing (“positive for”) the ovarian cancer disease state when the disease indicator is greater than a selected threshold or not evidencing (“negative for”) the ovarian cancer disease state when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 506 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for the ovarian cancer disease state if the biological sample evidences the ovarian cancer disease state based on the disease indicator. The diagnosis may be, for example, “negative” if the biological sample does not evidence the ovarian cancer disease state based on the disease indicator. A negative diagnosis may mean that the biological sample has a non-ovarian cancer state. The negative diagnosis for the ovarian cancer disease state can include at least one of a healthy state, a benign tumor state, or some other non-malignant state.

Generating the diagnosis output in step 506 may include determining that the score falls above (or at or above) a selected threshold and generating a positive diagnosis for the ovarian cancer disease state. Alternatively, step 506 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 506 may include a treatment output if the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

Table 1 below lists a first group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of screening for malignant pelvic tumors). The first group of peptide structures is listed in Table 1 in order with respect to relative significance to the disease indicator. In training, testing, and predictive use of this model, the quantification metrics for peptide structure PS-9, peptide structure PS-10, or a combination of the two may form one input. Table 1 also identifies check markers CK-1 and CK-2, which may also be used by the model.

TABLE 1
1^{st} Group of Peptide Structures Associated with Ovarian Cancer
(may be used to distinguish between malignant pelvic tumor (e.g., EOC) and healthy)

| | | | | | | Linking | |
|---|---|---|---|---|---|---|---|
| | | | | Mono- | Linking | Site Pos. | |
| | Peptide | (Protein) | (Peptide) | isotopic | Site Pos. | in | Glycan |
| PS-ID | Structure (PS) | SEQ ID | SEQ ID | mass | in Protein | Peptide | Structure |
| NO. | NAME | NO. | NO. | (Da) | Sequence | Sequence | GL NO. |
| PS-1 | ZA2G_128_5402 | 1 | 11 | 3342.26 | 128 | 8 | 5402 |
| PS-2 | IC1_253_6503 | 2 | 12 | 4961.09 | 253 | 4 | 6503 |
| PS-3 | CFAI_494_5402 | 3 | 13 | 3025.18 | 494 | 4 | 5402 |
| PS-4 | CERU_138_6513 | 4 | 14 | 4898.89 | 138 | 10 | 6513 |
| PS-5 | IGG1_297_3410 | 5 | 15 | 2633.04 | 180 | 5 | 3410 |
| PS-6 | HEMO_64_5402 | 6 | 16 | 4731.84 | 64 | 15 | 5402 |
| PS-7 | APOB_983_5402 | 7 | 17 | 5754.34 | 983 | 16 | 5402 |
| PS-8 | HPT_207_121005 | 8 | 18 | 6888.63 | 207 | 5.9 | 121005 |
| CK-1 | FINC_ | N/A | N/A | N/A | N/A | N/A | N/A |
| | SYTITGLQPGTDYK | | | | | | |
| PS-9 | IGG3_297_3400 | 9 | 19 | 2470.99 | 227 | 5 | 3400 |
| PS-10 | IGG4_297_3400 | 10 | 20 | 2470.99 | 227 | 5 | 3400 |
| CK-2 | APOM_135_8500_ | N/A | N/A | N/A | N/A | N/A | N/A |
| | CHK | | | | | | |

Table 2 below lists a second group of peptide structures associated with malignant pelvic tumors (e.g., ovarian cancer such as EOC). One or more features (e.g., relative abundance, concentration, site occupancy) of these peptide structures may be used in the supervised machine learning model described above to generate a disease indicator that predicts the probability of malignancy (e.g., in the context of triaging to distinguish between malignant and benign pelvic tumors). The second group of peptide structures is listed in Table 2 in order with respect to relative significance to the disease indicator. Table 2 also identifies check markers CK-3 and CK-4, which may also be used by the model.

TABLE 2
2^{nd} Group of Peptide Structures Associated with Ovarian Cancer
(may be used to distinguish between malignant v. benign pelvic tumors)

| | | | | | | Linking | |
|---|---|---|---|---|---|---|---|
| | | | | Mono- | Linking | Site Pos. | |
| | Peptide | (Protein) | (Peptide) | isotopic | Site Pos. | in | Glycan |
| PS-ID | Structure (PS) | SEQ ID | SEQ ID | mass | in Protein | Peptide | Structure |
| NO. | NAME | NO. | NO. | (Da) | Sequence | Sequence | GL NO. |
| CK-3 | APOD_98_9800_ | N/A | N/A | N/A | N/A | N/A | N/A |
| | CHECK | | | | | | |
| PS-11 | CO2_621_5200 | 20 | 31 | 2670.19 | 621 | 11 | 5200 |
| PS-5 | IGG1_297_3410 | 5 | 15 | 2633.04 | 180 | 5 | 3410 |
| PS-12 | AGP1_93_7612 | 21 | 32 | 4995.98 | 93 | 7 | 7612 |
| PS-13 | AACT_271_7602 | 22 | 33 | 4686.91 | 271 | 4 | 7602 |
| PS-14 | A2MG_1424_5402 | 23 | 34 | 4366.95 | 1424 | 3 | 5402 |
| PS-15 | AACT_271_6513 | 22 | 33 | 4758.93 | 271 | 4 | 6513 |
| PS-16 | CERU_397_5402 | 4 | 35 | 4330.76 | 397 | 2 | 5402 |
| PS-17 | APOB_3411_5301 | 7 | 36 | 3316.40 | 3411 | 7 | 5301 |
| PS-18 | AACT_106_6513 | 22 | 37 | 5406.24 | 106 | 2 | 6513 |
| PS-19 | CERU_138_5402 | 4 | 14 | 4096.61 | 138 | 10 | 5402 |
| PS-20 | A1AT_107_6513 | 24 | 38 | 6697.87 | 107 | 14 | 6513 |
| PS-21 | AGP1_93_7602 | 21 | 32 | 4849.93 | 93 | 7 | 7602 |
| PS-22 | VTNC_242_6502 | 25 | 39 | 5341.22 | 242 | 1 | 6502 |
| PS-23 | IGG2_297_3510 | 26 | 40 | 2804.13 | 176 | 5 | 3510 |
| PS-24 | CFAH_882_5411 | 27 | 41 | 4079.71 | 882 | 15 | 5411 |
| CK-4 | APOM_135_8500_ | N/A | N/A | N/A | N/A | N/A | N/A |
| | CHECK | | | | | | |
| PS-25 | AGP1_103_8704 | 21 | 42 | 4657.74 | 103 | 2 | 8704 |
| PS-26 | IGG1_297_4300 | 5 | 15 | 2445.95 | 180 | 5 | 4300 |
| PS-27 | APOH_253_5401 | 28 | 43 | 3163.24 | 253 | 3 | 5401 |
| PS-28 | APOD_98_5411 | 29 | 44 | 4312.85 | 98 | 16 | 5411 |
| PS-29 | TRFE_630_5411 | 30 | 45 | 4573.85 | 630 | 9 | 5411 |
| PS-30 | CERU_138_6502 | 4 | 14 | 4461.74 | 138 | 10 | 6502 |
| PS-31 | A2MG_1424_5411 | 23 | 34 | 4221.91 | 1424 | 3 | 5411 |
| PS-32 | A2MG_55_5411 | 23 | 46 | 4455.96 | 55 | 9 | 5411 |
| PS-33 | TRFE_630_5412 | 30 | 45 | 4864.95 | 630 | 9 | 5412 |
| PS-34 | IGG2_297_4511 | 26 | 40 | 3257.28 | 176 | 5 | 4511 |

### VI.B.1 Exemplary Methodology-Based on Table 3

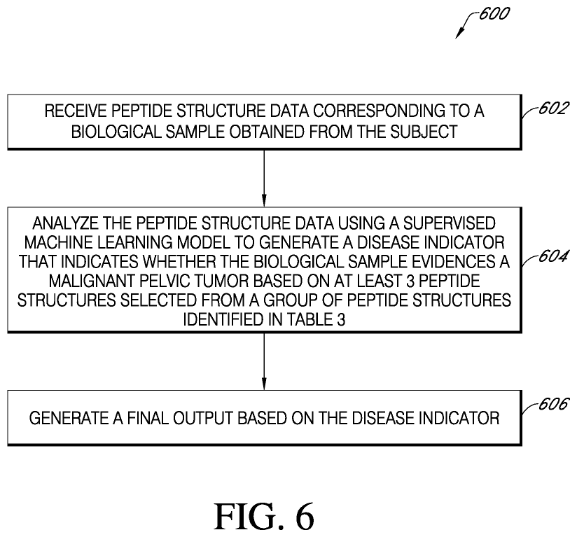

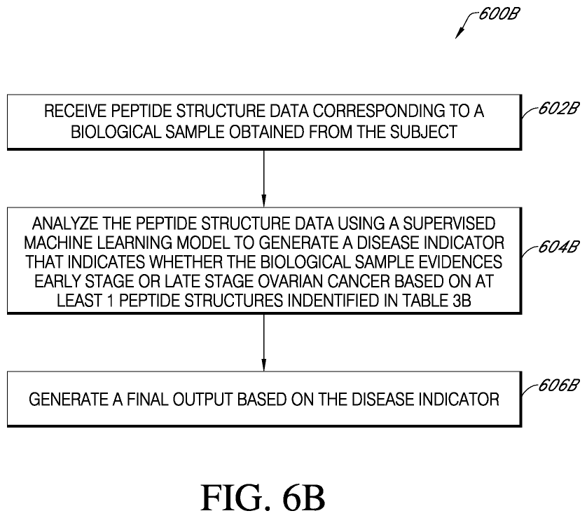

FIG. 6 is a flowchart of a process for diagnosing a subject with respect to an ovarian cancer disease state in accordance with one or more embodiments. Process 600 may be implemented using, for example, at least a portion of workflow 100 as described in FIGS. 1, 2A, and 2B and/or analysis system 300 as described in FIG. 3. Process 600 may be used to generate a final output that includes at least a diagnosis output for the subject.

Step 602 includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 3, with the peptide sequence being one of SEQ ID NOS: 11, 14, 15, 31, 32, 33, 34, 37, 38, 40, 42, 44, 45, 46, 53-65 in Table 3, the SEQ ID NOS being defined in Table 5 below.

Step 604 includes analyzing the peptide structure data using a supervised machine learning model to generate a disease indicator that predicts whether the biological sample evidences a malignant pelvic tumor or benign pelvic tumor based on at least three peptide structures selected from a group of peptide structures identified in Table 3. The group of peptide structures is listed in Table 3 with respect to relative significance to the disease indicator, which may be a probability score. In step 604, the group of peptide structures is associated with the malignancy (e.g., EOC). For example, the group of peptide structures in Table 3 includes peptide structures that have been determined relevant to distinguishing between a malignant and benign nature of a pelvic tumor.

In one or more embodiments, the at least 3 peptide structures includes at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-1, PS-5, PS-11, PS-15, PS-20, PS-25, PS-28, PS-29, PS-30, PS-31, PS-32, and PS-35 to PS-61 identified in Table 3.

In one or more embodiments, step 604 may be implemented using a binary classification model (e.g., a regression model). In some examples, the regression model may be, for example, penalized multivariable regression model. In various embodiments, the disease indicator may be computed using a weight coefficient associated with each peptide structure of the at least 3 peptide structures, the weight coefficient of a corresponding peptide structure of the at least 3 peptide structures may indicate the relative significance of the corresponding peptide structure to the disease indicator.

In some embodiments, step 604 may include computing a peptide structure profile for the biological sample that identifies a weighted value for each peptide structure of the at least 3 peptide structures. The weighted value for a peptide structure of the at least 3 peptide structures may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is evidences malignancy (e.g., EOC) and the supervised machine learning model is configured to generate an output that identifies the biological sample as either evidencing (“positive for”) malignancy when the disease indicator is greater than a selected threshold or not evidencing (“negative for”) malignancy when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 606 includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, “positive” for an ovarian cancer disease state (e.g., EOC) if the biological sample evidences malignancy based on the disease indicator. The diagnosis may be, for example, “negative” if the biological sample does not evidence malignancy based on the disease indicator. A negative diagnosis may mean that the biological sample evidences a benign status (or a non-ovarian cancer state).

Generating the diagnosis output in step 606 may include determining that the score falls above (or at or above) a selected threshold and generating a positive diagnosis for the ovarian cancer disease state. Alternatively, step 606 can include determining that the score falls below (or at or below) a selected threshold and generating a negative diagnosis for the ovarian cancer disease state. In some scoring systems, the score can include a probability score and the selected threshold can be 0.5. In other scoring systems, the selected threshold can fall within a range between 0.30 and 0.65.

In one or more embodiments, the final output in step 606 may include a treatment output if the disease indicator predicts malignancy and/or the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

TABLE 3
3^{rd} Group of Peptide Structures Associated with
Ovarian Cancer (may be used to distinguish
between malignant and benign pelvic tumors)

| | | (Pro- | (Pep- | Linking | |
|---|---|---|---|---|---|
| | | tein) | tide) | Site Pos. | Glycan |
| | | SEQ | SEQ | in | Structure |
| PS-ID | Peptide Structure | ID | ID | Protein | GL |
| NO. | (PS) NAME | NO. | NO. | Sequence | NO. |
| PS-35 | VTNC_169_5401 | 25 | 53 | 169 | 5401 |
| PS-36 | FETUA_176_6513 | 47 | 54 | 176 | 6513 |
| PS-37 | AGP1_93_7614 | 21 | 32 | 93 | 7614 |
| PS-38 | QUANTPEP.A2GL_ | 48 | 55 | N/A | N/A |
| | DLLLPQPDLR | | | | |
| PS-39 | HPT_184_5402 | 8 | 56 | 184 | 5402 |
| PS-40 | TRFE_432_6503 | 30 | 57 | 432 | 6503 |
| PS-41 | TRFE_630_6513 | 30 | 45 | 630 | 6513 |
| PS-42 | HEMO_453_5402 | 6 | 58 | 453 | 5402 |
| PS-43 | QUANTPEP.TTR_ | 49 | 59 | N/A | N/A |
| | TSESGELHGLT | | | | |
| | TEEEFVEGIYK | | | | |
| PS-5 | IGG1_297_3410 | 5 | 15 | 297 | 3410 |
| PS-44 | TRFE_630_5400 | 30 | 45 | 630 | 5400 |
| PS-45 | AGP1_103_9804 | 21 | 42 | 103 | 9804 |
| PS-46 | TRFE_432_6501 | 30 | 57 | 432 | 6501 |
| PS-47 | HPT_241_5402 | 8 | 60 | 241 | 5402 |
| PS-48 | IGG1_297_5510 | 5 | 15 | 297 | 5510 |
| PS-49 | QUANTPEP.AFAM_ | 50 | 61 | N/A | N/A |
| | SDVGFLPPFP | | | | |
| | TLDPEEK | | | | |
| PS-32 | A2MG_55_5411 | 23 | 46 | 55 | 5411 |
| PS-50 | IGG2_297_5510 | 26 | 40 | 297 | 5510 |
| PS-51 | AGP1_103_7603 | 21 | 42 | 103 | 7603 |
| PS-52 | IGG2_297_5400 | 26 | 40 | 297 | 5400 |
| PS-1 | ZA2G_128_5402 | | 11 | 128 | 5402 |
| PS-53 | TRFE_630_6502 | 30 | 45 | 630 | 6502 |
| PS-54 | TRFE_432_6502 | 30 | 57 | 432 | 6502 |
| PS-55 | IGG2_297_4510 | 26 | 40 | 297 | 4510 |
| PS-56 | AACT_106_7614 | 22 | 37 | 106 | 7614 |
| PS-57 | PEP-APOA1_ | 51 | 62 | N/A | N/A |
| | VSFLSALEEYTK | | | | |
| PS-11 | CO2_621_5200 | 20 | 31 | 621 | 5200 |
| PS-15 | AACT_271_6513 | 22 | 33 | 271 | 6513 |
| PS-58 | FETUA_176_5401 | 47 | 54 | 176 | 5401 |
| PS-59 | FETUA_346_1102 | 47 | 63 | 346 | 1102 |
| PS-60 | PEP-APOA1_ | 51 | 64 | N/A | N/A |
| | THLAPYSDELR | | | | |
| PS-29 | TRFE_630_5411 | 30 | 45 | 630 | 5411 |
| PS-25 | AGP1_103_8704 | 21 | 42 | 103 | 8704 |
| PS-30 | CERU_138_6502 | 4 | 14 | 138 | 6502 |
| PS-20 | A1AT_107_6513 | 24 | 38 | 107 | 6513 |
| PS-31 | A2MG_1424_5411 | 23 | 34 | 1424 | 5411 |
| PS-28 | APOD_98_5411 | 29 | 44 | 98 | 5411 |
| PS-61 | C4BPA_221_5402 | 52 | 65 | 221 | 5402 |

### VI.B.2 Exemplary Methodology-Based on Table 3B

FIG. 6B is a flowchart of a process for diagnosing a subject with respect to an ovarian cancer disease state in accordance with one or more embodiments. Process 600B may be implemented using, for example, at least a portion of workflow 100 as described in FIGS. 1, 2A, and 2B and/or analysis system 300 as described in FIG. 3. Process 600B may be used to generate a final output that includes at least a diagnosis output for the subject such as, for example early stage EOC or late stage EOC.

Step 602B includes receiving peptide structure data corresponding to a biological sample obtained from the subject. The peptide structure data may be, for example, one example of an implementation of peptide structure data 310 in FIG. 3. The peptide structure data may include quantification data for each peptide structure of a plurality of peptide structures. The quantification data may include, for example, one or more quantification metrics for each peptide structure of the plurality of peptide structures. A quantification metric for a peptide structure may be, for example, but is not limited to, a relative quantity, an adjusted quantity, a normalized quantity, a relative concentration, an adjusted concentration, or a normalized concentration. In this manner, the quantification data for a given peptide structure provides an indication of the abundance of the peptide structure in the biological sample. In some cases, at least one peptide structure includes a glycopeptide structure having a peptide sequence and a glycan structure linked to the peptide sequence at a linking site of the peptide sequence, as identified in Table 3B, with the peptide sequence being one of SEQ ID NOS: 14, 18, 32, 33, 37, 39, 42, 45, 54, 56, 60, 68-77 in Table 3B, the SEQ ID NOS being defined in Table 5 below. It should be noted that the glycopeptides of Table 3B were part of a glycoprotein that are further described in Table 6 and that the glycan portion of the glycopeptides is described in Table 7.

Step 604B includes analyzing the peptide structure data using a supervised machine learning model to generate a disease indicator that predicts whether the biological sample evidences an early stage or late stage EOC on at least one peptide structure selected from a group of peptide structures identified in Table 3B. In step 604B, the group of peptide structures is associated with the early stage or late stage EOC. For example, the group of peptide structures in Table 3B includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, or all 36 of the peptide structures PS-4, PS-8, PS-18, PS-36, PS-37, PS-41, PS-56, PS-62 to PS-90 identified in Table 3B.

In one or more embodiments, step 604B may be implemented using a binary classification model (e.g., a regression model). In some examples, the regression model may be, for example, penalized multivariable regression model. In various embodiments, the disease indicator may be computed using a weight coefficient associated with each peptide structure of the at least 1 peptide structure, the weight coefficient of a corresponding peptide structure of the at least 1 peptide structures may indicate the relative significance of the corresponding peptide structure to the disease indicator.

In some embodiments, step 604B may include computing a peptide structure profile for the biological sample that identifies a weighted value for each peptide structure of the at least 1 peptide structure. The weighted value for a peptide structure of the at least 1 peptide structure may be a product of a quantification metric for the peptide structure identified from the peptide structure data and a weight coefficient for the peptide structure. The disease indicator may be computed using the peptide structure profile. For example, the disease indicator may be a logit equal to the sum of the weighted values for the peptide structures plus an intercept value. The intercept value may be determined during the training of the model.

In various embodiments, the disease indicator comprises a probability that the biological sample is evidences malignancy (e.g., EOC) and the supervised machine learning model is configured to generate an output that identifies the biological sample as either evidencing (“positive for”) malignancy when the disease indicator is greater than a selected threshold or not evidencing (“negative for”) malignancy when the disease indicator is not greater than the selected threshold. The selected threshold may be, for example, 0.30, 0.35, 0.40, 0.45, 0.50, 0.55, 0.60, or some other threshold between 0.30 and 0.65. In one or more embodiments, the selected threshold is 0.5.

Step 606B includes generating a final output based on the disease indicator. The final output may include a diagnosis output, such as, for example, diagnosis output 324 in FIG. 3. The diagnosis output may include the disease indicator, or a diagnosis made based on the disease indicator. The diagnosis may be, for example, early stage or late stage based on the disease indicator. An early stage diagnosis may mean that the biological sample evidences a stage 1 or 2 EOC. A late stage diagnosis may mean that the biological sample evidences a stage 3 or 4 EOC.

Generating the diagnosis output in step 606B may include determining that the score falls above (or at or above) a selected threshold and generating a positive diagnosis for the late stage ovarian cancer disease state. Alternatively, step 606B can include determining that the score falls below (or at or below) a selected threshold and generating a negative diagnosis for the late stage ovarian cancer disease state. In some scoring systems, the score can include a probability score and the selected threshold can be 0.5. In other scoring systems, the selected threshold can fall within a range between 0.30 and 0.65.

In one or more embodiments, the final output in step 606B may include a treatment output if the disease indicator predicts malignancy and/or the diagnosis output indicates a positive diagnosis for the ovarian cancer disease state. The treatment output may include, for example, at least one of an identification of a treatment for the subject, a treatment plan for administering the treatment, or both. Treatment for ovarian cancer may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment plan may include, for example, but is not limited to, a timeline or schedule for administering the treatment, dosing information, other treatment-related information, or a combination thereof.

TABLE 3B
Group of Peptide Structures Associated with Ovarian Cancer
(may be used to distinguish between early stage v. late stage ovarian cancer)

| | | | | | | Linking | |
|---|---|---|---|---|---|---|---|
| | | | | Mono- | Linking | Site Pos. | |
| | | (Protein) | (Peptide) | isotopic | Site Pos. | in | Glycan |
| PS-ID | Peptide Structure | SEQ ID | SEQ ID | mass | in Protein | Peptide | Structure |
| NO. | (PS) NAME | NO. | NO. | (Da) | Sequence | Sequence | GL NO. |
| PS-62 | AACT_106_6503 | 22 | 37 | 5260.186906 | 106 | 2 | 6503 |
| PS-18 | AACT_106_6513 | 22 | 37 | 5406.244812 | 106 | 2 | 6513 |
| PS-63 | AACT_106_7604 | 22 | 37 | 5916.414504 | 106 | 2 | 7604 |
| PS-56 | AACT_106_7614 | 22 | 37 | 6062.47241 | 106 | 2 | 7614 |
| PS-64 | AACT_271_6502 | 22 | 33 | 4321.777556 | 271 | 4 | 6502 |
| PS-65 | AGP1_103_6503 | 21 | 42 | 3636.3824 | 103 | 2 | 6503 |
| PS-66 | AGP1_103_6513 | 21 | 42 | 3782.440306 | 103 | 2 | 6513 |
| PS-67 | AGP1_103_7602 | 21 | 42 | 3710.419178 | 103 | 2 | 7602 |
| PS-68 | AGP1_103_7614 | 21 | 42 | 4438.667904 | 103 | 2 | 7614 |
| PS-69 | AGP1_93_6503 | 21 | 32 | 4775.889354 | 93 | 7 | 6503 |
| PS-70 | AGP1_93_6513 | 21 | 32 | 4921.94726 | 93 | 7 | 6513 |
| PS-71 | AGP1_93_7613 | 21 | 32 | 5287.079448 | 93 | 7 | 7613 |
| PS-37 | AGP1_93_7614 | 21 | 32 | 5578.174858 | 93 | 7 | 7614 |
| PS-72 | AGP12_56_6503 | 21, 66 | 68 | 3656.339868 | 56 | 5 | 6503 |
| PS-73 | AGP12_56_6513 | 21, 66 | 68 | 3802.397774 | 56 | 5 | 6513 |
| PS-74 | AGP12_72_6503 | 21, 66 | 69 | 4779.946464 | 72 | 15 | 6503 |
| PS-75 | AGP12_72_7603 | 21, 66 | 69 | 5145.078652 | 72 | 15 | 7603 |
| PS-76 | AGP2_103_6513 | 66 | 70 | 3768.424656 | 103 | 2 | 6513 |
| PS-77 | APOH_162_6503 | 28 | 71 | 4328.746982 | 162 | 8 | 6503 |
| PS-4 | CERU_138_6513 | 4 | 14 | 4898.89152 | 138 | 10 | 6513 |
| PS-78 | CERU_397_6513 | 4 | 72 | 5133.049472 | 397 | 2 | 6513 |
| PS-79 | CERU_762_6513 | 4 | 73 | 5028.0545 | 762 | 9 | 6513 |
| PS-80 | FETUA_156_6503 | 47 | 74 | 4631.839236 | 156 | 12 | 6503 |
| PS-36 | FETUA_176_6513 | 47 | 54 | 5371.203674 | 176 | 11 | 6513 |
| PS-81 | HEMO_187_6503 | 6 | 75 | 4264.661538 | 187 | 7 | 6503 |
| PS-82 | HEMO_187_6513 | 6 | 75 | 4410.719444 | 187 | 7 | 6513 |
| PS-83 | HPT_184_6513 | 8 | 56 | 5685.442854 | 184 | 6 | 6513 |
| PS-84 | HPT_207_11904 | 8 | 18 | 6232.403228 | 207 | 5, 9 | 5402 or |
| | | | | | | | 6502 |
| PS-85 | HPT_207_11915 | 8 | 18 | 6669.556544 | 207, 211 | 5, 9 | 5402 or |
| | | | | | | | 6513 |
| PS-8 | HPT_207_121005 | 8 | 18 | 6888.630826 | 207 | 5, 9 | 6502 or |
| | | | | | | | 6503 |
| PS-86 | HPT_241_6513 | 8 | 60 | 4801.061826 | 241 | 6 | 6513 |
| PS-87 | HPT_241_7613 | 8 | 60 | 5166.194014 | 241 | 6 | 7613 |
| PS-88 | KNG1_205_6513 | 67 | 76 | 4419.754828 | 205 | 9 | 6513 |
| PS-89 | KNG1_294_6503 | 67 | 77 | 4291.682988 | 294 | 6 | 6503 |
| PS-41 | TRFE_630_6513 | 30 | 45 | 5521.174696 | 630 | 9 | 6513 |
| PS-90 | VTNC_242_6503 | 25 | 39 | 5632.31501 | 242 | 1 | 6503 |

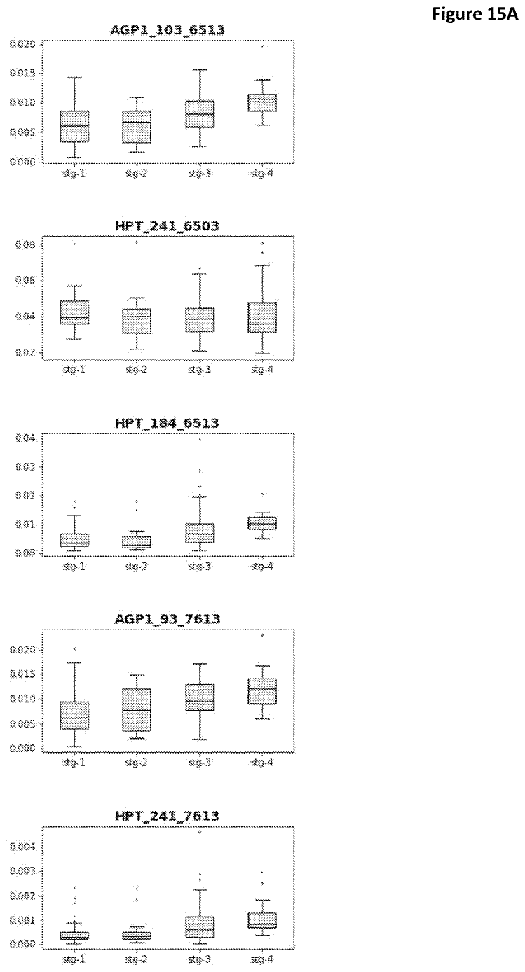

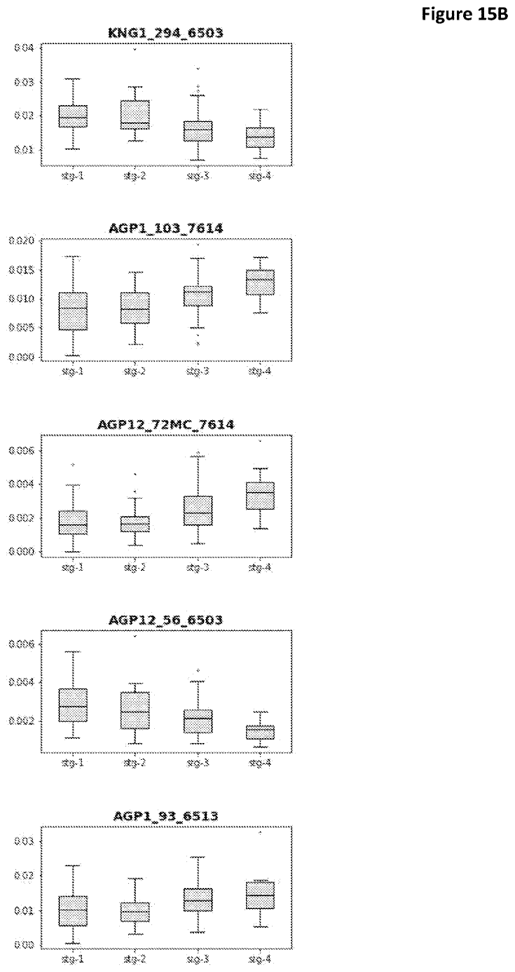

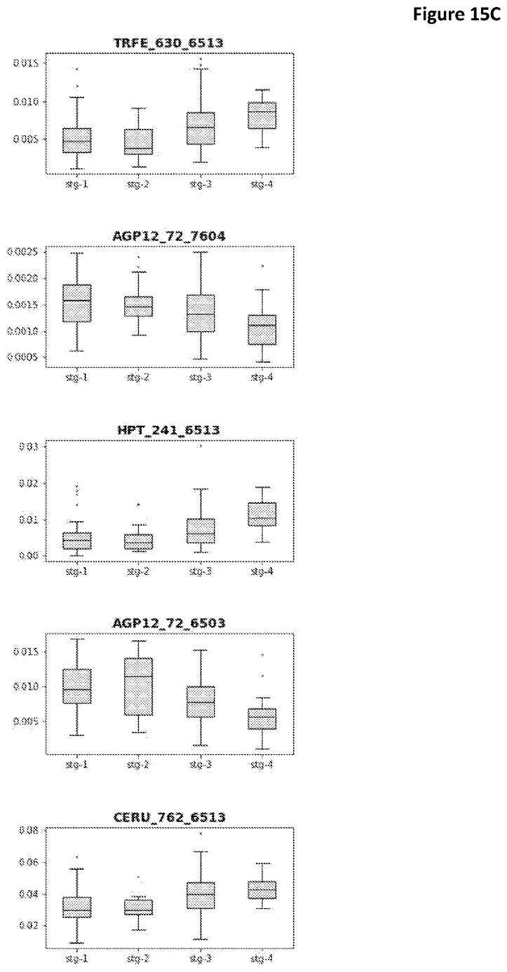

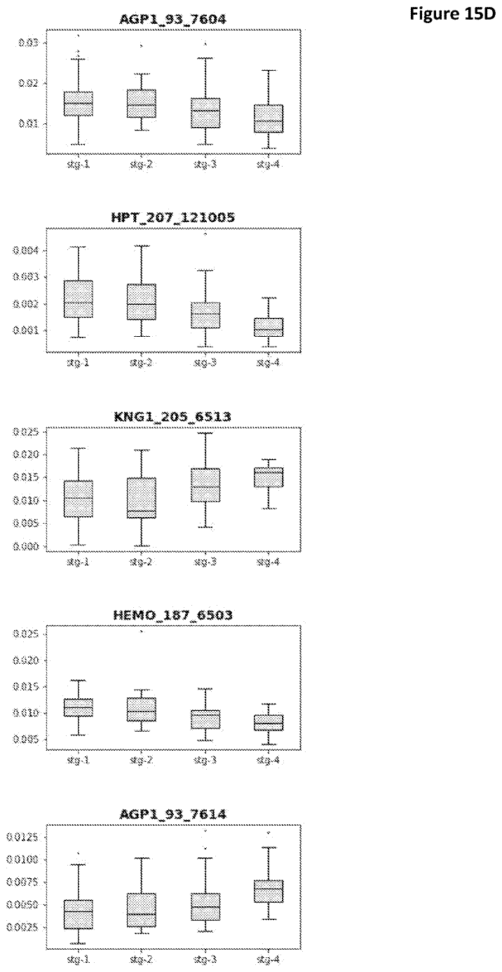

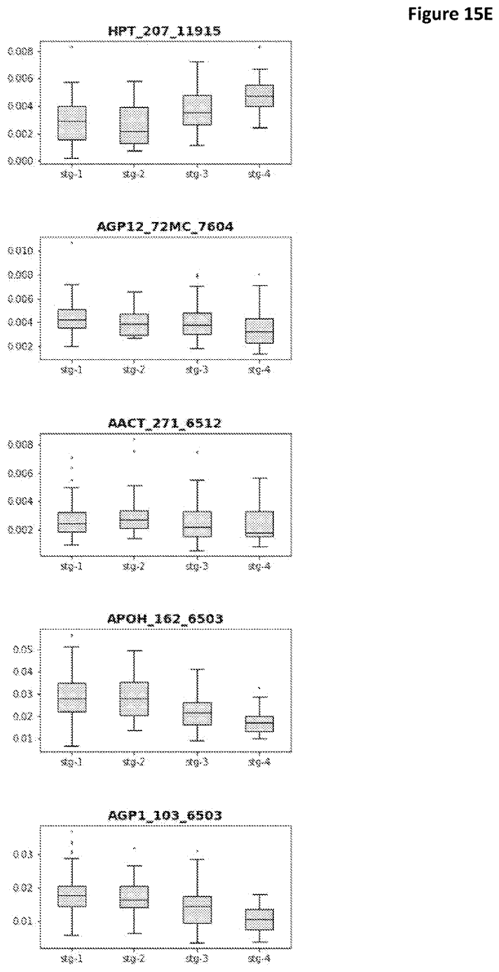

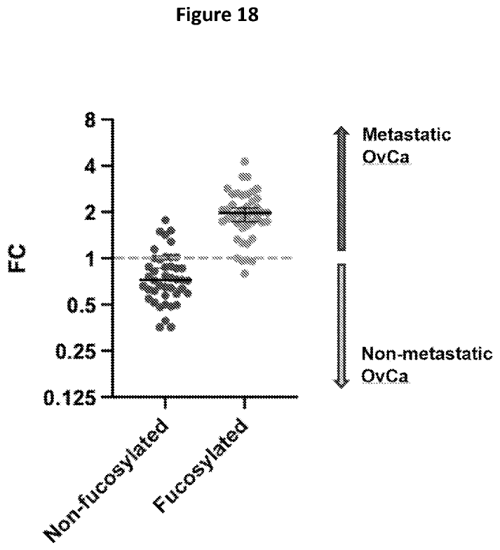

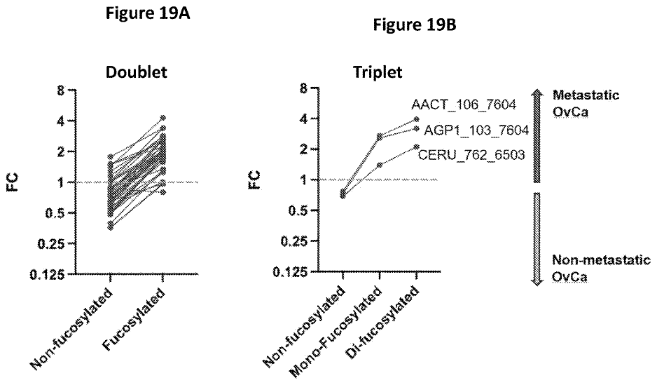

It is worthwhile to note that with a few exceptions (PS-62 and PS-67), the majority of glycopeptides were tri- and tetra-antennary glycans with or without a fucose and were found to be associated with either early stage or late stage EOC. Fold changes (FCs) for several glycopeptides in stage IV (referred to as metastatic ovarian cancer) vs benign/stage I/II/III (referred to as non-metastatic ovarian cancer) were calculated by normalizing to normal blood samples, as illustrated in FIG. 18. The FCs were observed to stratify between fucosylated and non-fucosylated (plots include median and 95% confidence interval). FCs that were above the 1 corresponded to markers that correlate with metastatic ovarian cancer and those below 1 corresponded to markers that correlate with non-metastatic ovarian cancer. The Wilcoxon matched-pairs signed rank test was used to compare the two groups and a p value found to be <0.0001 showing a statistical difference between non-fucosylated and fucosylated. FIGS. 19A and 19B illustrate that a same set of markers in doublets/triplets analysis for fucosylation revealed a strong association with either metastatic ovarian cancer or non-metastatic ovarian cancer. Doublet analysis refers to monitoring the fold change of a non-fucosylated and fucosylated glycopeptide that was tri or tetra-antennary for sialic acid and had the same peptide sequence and glycan linking site. Triplet analysis refers to monitoring the fold change of a non-fucosylated, fucosylated, and di-fucosylated glycopeptide that was tri or tetraantennary for sialic acid and had the same peptide sequence and glycan linking site. FIGS. 15A-15E shows that the fucosylated biomarkers (have a number 1 in the 2^{nd} to last number in the Peptide structure (PS) Name) show a relatively upward trend from stage 1/2 to stage 3/4. In contrast, FIGS. 15A-15E shows that the non-fucosylated biomarkers (have a number 0 in the 2^{nd} to last number in the Peptide structure (PS) Name) show an relatively downward trend from stage 1/2 to stage 3/4. For instance, the glycan numbers 6513, 7613, 7614 are examples of fucosylated glycans having tri or tetra-antennary sialic acids. The glycan numbers 6503, 7603, 7604 are examples of non-fucosylated glycans having tri or tetra-antennary sialic acids.

### VI.B.3 Exemplary Methodology—Based on Table 3C

In another embodiment, process 600B may be implemented using Table 3C instead of Table 3B. In some cases, at least one peptide structure includes a glycopeptide structure having a peptide sequence and a glycan structure linked to the peptide sequence at a linking site of the peptide sequence, as identified in Table 3C, with the peptide sequence being one of SEQ ID NOS: 101-125 in Table 3C.

The group of peptide structures in Table 3C includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide SEQ ID NOS: 101-125 identified in Table 3C.

TABLE 3C
Group of Peptide Structures Associated with Ovarian Cancer
(may be used to distinguish between early state v. late stage ovarian cancer)

| | | | | Linking | |
|---|---|---|---|---|---|
| | | | Linking | Site | |
| | | | Site | Position | |
| SEQ | | | Position | in | Glycan |
| ID | Peptide Structure | | in Protein | Peptide | Structure |
| NO | (PS) NAME | Peptide Sequence | Sequence | Sequence | GL NO. |
| 101 | AACT_271_6512 | YTGNASALFILPDQDK | 271 | 4 | 6512 |
| 102 | AGP1_103_6503 | ENGTISR | 103 | 2 | 6503 |
| 103 | AGP_103_6513 | ENGTISR | 103 | 2 | 6513 |
| 104 | AGP1_103_7614 | ENGTISR | 103 | 2 | 7614 |
| 105 | AGP1_93_6513 | QDQCIYNTTYLNVQR | 93 | 7 | 6513 |
| 106 | AGP_93_7604 | QDQCIYNTTYLNVQR | 93 | 7 | 7604 |
| 107 | AGP_93_7613 | QDQCIYNTTYLNVQR | 93 | 7 | 7613 |
| 108 | AGP_93_7614 | QDQCIYNTTYLNVQR | 93 | 7 | 7614 |
| 109 | AGP12_56_6503 | NEEYNK | 56 | 5 | 6503 |
| 110 | AGP12_72_6503 | SVQEIQATFFYFTPNK | 72 | 15 | 6503 |
| 111 | AGP12_72_7604 | SVQEIQATFFYFTPNK | 72 | 15 | 7604 |
| 112 | AGP12_72MC_7604 | SVQEIQATFFYFTPNKTEDTIFLR | 72 | 15 | 7604 |
| 113 | AGP12_72MC_7614 | SVQEIQATFFYFTPNKTEDTIFLR | 72 | 15 | 7614 |
| 114 | APOH_162_6503 | VYKPSAGNNSLYR | 162 | 8 | 6503 |
| 115 | CERU_762_6513 | ELHHLQEQNVSNAFLDK | 762 | 9 | 6513 |
| 116 | HEMO_187_6503 | SWPAVGNCSSALR | 187 | 7 | 6503 |
| 117 | HPT_184_6513 | MVSHHNLTTGATLINEQWLLTTA | 184 | 6 | 6513 |
| | | K | | | |
| 118 | HPT_207_11915 | NLFLNHSENATAK | 207 | 5,9 | 5402 or |
| | | | | | 6503 |
| 119 | HPT_207_121005 | NLFLNHSENATAK | 207 | 5,9 | 6502 or |
| | | | | | 6503 |
| 120 | HPT_241_6503 | VVLHPNYSQVDIGLIK | 241 | 6 | 6503 |
| 121 | HPT_241_6513 | VVLHPNYSQVDIGLIK | 241 | 6 | 6513 |
| 122 | HPT_241_7613 | VVLHPNYSQVDIGLIK | 241 | 6 | 7613 |
| 123 | KNG1_205_6513 | ITYSIVQTNCSK | 205 | 9 | 6513 |
| 124 | KNG1_294_6503 | LNAENNATFYFK | 294 | 6 | 6503 |
| 125 | TRFE_630_6513 | QQQHLFGSNVTDCSGNFCLFR | 630 | 9 | 6513 |

In Table 3C, the first three or four characters before the first underscore of the peptide structure (PS) name corresponds to the abbreviation of the protein name. More details on the protein sequence can be found in Table 6B below.

### VI.B.3 Exemplary Methodology—Based on Table 3D

In another embodiment, process 600B may be implemented using Table 3D instead of Table 3B. In some cases, at least one peptide structure includes a glycopeptide structure having a peptide sequence and a glycan structure linked to the peptide sequence at a linking site of the peptide sequence, as identified in Table 3D, with the peptide sequence being one of SEQ ID NOS: 126-175 in Table 3D. In other cases, at least one peptide structure includes a glycopeptide structure having a peptide sequence and a glycan structure linked to the peptide sequence at a linking site of the peptide sequence, as identified in Table 3D, with the peptide sequence being one of SEQ ID NOS: 131-134, 137, 139, 140, 143, 151, 165-167 in Table 3D.

The group of peptide structures in Table 3D includes peptide structures that have been determined relevant to distinguishing between early stage (stages 1 and 2) or late stage (stages 3 and 4) EOC.

In one or more embodiments, the at least 1 peptide structure includes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or all 50 of the peptide SEQ ID NOS: 126-175 identified in Table 3D.

TABLE 3D
Group of Peptide Structures Associated with Ovarian Cancer
(may be used to distinguish between early state v. late stage ovarian cancer)

| | | | | | Linking | Linking | | |
|---|---|---|---|---|---|---|---|---|
| | | | Prot | Pep | Site Pos. | Site Pos. | | |
| PS- | | | SEQ | SEQ | in | in | Glycan | |
| ID | | | ID | ID | Protein | Peptide | Structure | Monoisotopic |
| NO. | PS-Name | Protein Name | NO. | NO. | Seq | Seq | GL NO. | mass (Da) |
| 91 | A1AT_107_ | Alpha-1- | 24 | 126 | 107 | 14 | 6512 | 6406.779 |
| | 6512 | antitrypsin | | | | | | |
| 92 | A2MG_1424_ | Alpha-2- | 23 | 127 | 1424 | 3 | 6501 | 4440.983 |
| | 6501 | macroglobulin | | | | | | |
| 93 | A2MG_1424_ | Alpha-2- | 23 | 128 | 1424 | 3 | 6511 | 4587.041 |
| | 6511 | macroglobulin | | | | | | |
| 94 | AACT_106_ | Alpha-1- | 22 | 129 | 106 | 2 | 6503 | 5260.187 |
| | 6503 | antichymotrypsin | | | | | | |
| 95 | AACT_106_ | Alpha-1- | 22 | 130 | 106 | 2 | 7604 | 5916.415 |
| | 7604 | antichymotrypsin | | | | | | |
| 96 | AACT_106_ | Alpha-1- | 22 | 131 | 106 | 2 | 7614 | 6062.472 |
| | 7614 | antichymotrypsin | | | | | | |
| 97 | AACT_271_ | Alpha-1- | 22 | 132 | 271 | 4 | 6502 | 4321.778 |
| | 6502 | antichymotrypsin | | | | | | |
| 98 | AACT_271_ | Alpha-1- | 22 | 133 | 271 | 4 | 6503 | 4612.873 |
| | 6503 | antichymotrypsin | | | | | | |
| 99 | AGP12_56_ | Alpha-1-acid | 21 or | 134 | 56 | 5 | 6503 | 3656.34 |
| | 6503 | glycoprotein 1 | 66 | | | | | |
| | | and/or 2 | | | | | | |
| 100 | AGP12_72MC_ | Alpha-1-acid | 21 or | 135 | 72 | 15 | 6503 | 5755.449 |
| | 6503 | glycoprotein 1 | 66 | | | | | |
| | | and/or 2 | | | | | | |
| 101 | AGP12_72MC_ | Alpha-1-acid | 21 or | 136 | 72 | 15 | 6513 | 5901.507 |
| | 6513 | glycoprotein 1 | 66 | | | | | |
| | | and/or 2 | | | | | | |
| 102 | AGP12 72MC_ | Alpha-1-acid | 21 or | 137 | 72 | 15 | 7613 | 6266.639 |
| | 7613 | glycoprotein 1 | 66 | | | | | |
| | | and/or 2 | | | | | | |
| 103 | AGP12_72MC_ | Alpha-1-acid | 21 or | 138 | 72 | 15 | 7614 | 6557.734 |
| | 7614 | glycoprotein 1 | 66 | | | | | |
| | | and/or 2 | | | | | | |
| 104 | AGP12_72_ | Alpha-1-acid | 21 or | 139 | 72 | 15 | 6503 | 4779.946 |
| | 6503 | glycoprotein 1 | 66 | | | | | |
| | | and/or 2 | | | | | | |
| 105 | AGP12_72_ | Alpha-1-acid | 21 or | 140 | 72 | 15 | 7603 | 5145.079 |
| | 7603 | glycoprotein 1 | 66 | | | | | |
| | | and/or 2 | | | | | | |
| 106 | AGP12_72_ | Alpha-1-acid | 21 or | 141 | 72 | 15 | 7604 | 5436.174 |
| | 7604 | glycoprotein 1 | 66 | | | | | |
| | | and/or 2 | | | | | | |
| 107 | AGP1_103_ | Alpha-1-acid | 21 | 142 | 103 | 2 | 6503 | 3636.382 |
| | 6503 | glycoprotein 1 | | | | | | |
| 108 | AGP1_103_ | Alpha-1-acid | 21 | 143 | 103 | 2 | 6513 | 3782.44 |
| | 6513 | glycoprotein 1 | | | | | | |
| 109 | AGP1_103_ | Alpha-1-acid | 21 | 144 | 103 | 2 | 7602 | 3710.419 |
| | 7602 | glycoprotein 1 | | | | | | |
| 110 | AGP1_103_ | Alpha-1-acid | 21 | 145 | 103 | 2 | 7604 | 4292.61 |
| | 7604 | glycoprotein 1 | | | | | | |
| 111 | AGP1_103_ | Alpha-1-acid | 21 | 146 | 103 | 2 | 7614 | 4438.668 |
| | 7614 | glycoprotein 1 | | | | | | |
| 112 | AGP1_93_ | Alpha-1-acid | 21 | 147 | 93 | 7 | 6503 | 4775.889 |
| | 6503 | glycoprotein 1 | | | | | | |
| 113 | AGP1_93_ | Alpha-1-acid | 21 | 148 | 93 | 7 | 7602 | 4849.926 |
| | 7602 | glycoprotein 1 | | | | | | |
| 114 | AGP1_93_ | Alpha-1-acid | 21 | 149 | 93 | 7 | 7603 | 5141.022 |
| | 7603 | glycoprotein 1 | | | | | | |
| 115 | AGP1_93_ | Alpha-1-acid | 21 | 150 | 93 | 7 | 7604 | 5432.117 |
| | 7604 | glycoprotein 1 | | | | | | |
| 116 | AGP1_93_ | Alpha-1-acid | 21 | 151 | 93 | 7 | 7612 | 4995.984 |
| | 7612 | glycoprotein 1 | | | | | | |
| 117 | AGP1_93_ | Alpha-1-acid | 21 | 152 | 93 | 7 | 7613 | 5287.079 |
| | 7613 | glycoprotein 1 | | | | | | |
| 118 | AGP1_93_ | Alpha-1-acid | 21 | 153 | 93 | 7 | 7614 | 5578.175 |
| | 7614 | glycoprotein 1 | | | | | | |
| 119 | AGP2_103_ | Alpha-1-acid | 66 | 154 | 103 | 2 | 6503 | 3622.367 |
| | 6503 | glycoprotein 2 | | | | | | |
| 120 | AGP2_103_ | Alpha-1-acid | 66 | 155 | 103 | 2 | 6513 | 3768.425 |
| | 6513 | glycoprotein 2 | | | | | | |
| 121 | AGP2_103_ | Alpha-1-acid | 66 | 156 | 103 | 2 | 7604 | 4278.594 |
| | 7604 | glycoprotein 2 | | | | | | |
| 122 | APOH_162_ | Beta-2- | 28 | 157 | 162 | 8 | 6503 | 4328.747 |
| | 6503 | glycoprotein1 | | | | | | |
| 123 | APOH_253_ | Beta-2- | 28 | 158 | 253 | 3 | 5412 | 3600.389 |
| | 5412 | glycoprotein1 | | | | | | |
| 124 | CERU_397_ | Ceruloplasmin | 4 | 159 | 397 | 2 | 6513 | 5133.049 |
| | 6513 | | | | | | | |
| 125 | CERU_762_ | Ceruloplasmin | 4 | 160 | 762 | 9 | 6503 | 4881.997 |
| | 6503 | | | | | | | |
| 126 | FETUA_156_ | Alpha-2-HS- | 47 | 161 | 156 | 12 | 6503 | 4631.839 |
| | 6503 | glycoprotein | | | | | | |
| 127 | FETUA_156_ | Alpha-2-HS- | 47 | 162 | 156 | 12 | 6513 | 4777.897 |
| | 6513 | glycoprotein | | | | | | |
| 128 | FETUA_176_ | Alpha-2-HS- | 47 | 163 | 176 | 11 | 6513 | 5371.204 |
| | 6513 | glycoprotein | | | | | | |
| 129 | HEMO_187_ | Hemopexin | 6 | 164 | 187 | 7 | 6503 | 4264.662 |
| | 6503 | | | | | | | |
| 130 | HEMO_187_ | Hemopexin | 6 | 165 | 187 | 7 | 6513 | 4410.719 |
| | 6513 | | | | | | | |
| 131 | HPT_184_ | Haptoglobin | 8 | 166 | 184 | 6 | 6513 | 5685.443 |
| | 6513 | | | | | | | |
| 132 | HPT_207_ | Haptoglobin | 8 | 167 | 207 or | 5 or 9 | 5402 or | 6232.403 |
| | 11904 | | | | 211 | | 6502 | |
| 133 | HPT_207_ | Haptoglobin | 8 | 168 | 207 or | 5 or 9 | 5402 or | 6378.461 |
| | 11914 | | | | 211 | | 6512 | |
| 134 | HPT_207_ | Haptoglobin | 8 | 169 | 207 or | 5 or 9 | 5402 or | 6669.557 |
| | 11915 | | | | 211 | | 6513 | |
| 135 | HPT_207_ | Haptoglobin | 8 | 170 | 207 or | 5 or 9 | 6502 or | 6888.631 |
| | 121005 | | | | 211 | | 6503 | |
| 136 | HPT_241_ | Haptoglobin | 8 | 171 | 241 | 6 | 6511 | 4218.871 |
| | 6511 | | | | | | | |
| 137 | HPT_241_ | Haptoglobin | 8 | 172 | 241 | 6 | 6512 | 4509.966 |
| | 6512 | | | | | | | |
| 138 | HPT_241_ | Haptoglobin | 8 | 173 | 241 | 6 | 6513 | 4801.062 |
| | 6513 | | | | | | | |
| 139 | HPT_241_ | Haptoglobin | 8 | 174 | 241 | 6 | 7613 | 5166.194 |
| | 7613 | | | | | | | |
| 140 | KNG1_294_ | Kininogen-1 | 67 | 175 | 294 | 6 | 6503 | 4291.683 |
| | 6503 | | | | | | | |

With respect to Tables 1, 2, 3, 3A, 3B, 3C, and 3D, they include the Peptide Structure (PS) Name (e.g., KNG1_294_6503), which is a reference code for the protein name (e.g., KNG1), followed by the glycan linking site position in the protein (e.g., the number 294 that is preceded by an underscore and represents a sequential amino acid position in protein KNG1), and followed by the glycan structure GL number (e.g., the number 6503 that is preceded by an underscore and represents a glycan composition Hex(6)HexNAc(5)Fuc(0)NeuAc(3)). The Peptide Structure (PS) Name of contains a prefix that represents an abbreviation (that may include a combination of letters and numbers) for a protein abbreviation that corresponds to the Protein Abbreviation of Table 6. The term Linking Site Pos. in Protein Sequence is a number that refers to the sequential position of an amino acid of the corresponding protein in which a glycan is attached. For the Linking Site Pos. in Protein Sequence, the amino acid position of the peptide sequence is defined by the sequentially numbered order of amino acids based on the Uniprot ID of the corresponding protein for the peptide sequence. The term Linking Site Pos. in Peptide Sequence is a number that refers to the sequential position of an amino acid of the corresponding peptide in which a glycan is attached. For the Linking Site Pos. in peptide Sequence, the amino acid position of the peptide sequence is defined by the sequentially numbered order of amino acids for the peptide sequence. The term Glycan Structure GL No. is a number that corresponds to a symbol structure and a composition of the glycan as indicated in Tables 7. In some embodiments, the term AGP12 for SEQ ID NOs: 68-69 represents that the glycopeptide is a fragment of either AGP1 or AGP2.

In some instances, if the first number subsequent to the first underscore in the Peptide Structure (PS) NAME is inconsistent with the Glycan Linking Site Pos. in Protein Sequence column, then the Glycan Linking Site Pos. in Protein Sequence column should be used for identification of the peptide. In some instances, if the second number subsequent to the second underscore in the Peptide Structure (PS) NAME is inconsistent with the Glycan Structure GL NO column, then the Glycan Structure GL NO column should be used for identification of the glycan portion of the glycopeptide. If the Peptide Structure (PS) NAME does not contain any numbers, then the peptide is non-glycosylated. In some instances of the Peptide Structure (PS) NAME, subsequent to the prefix, there is a number noted with the notation MC that indicates that there was a missed cleavage at position in the peptide sequence as noted by the number.

### VI.C. Training a Model to Predict Ovarian Cancer (e.g., Epithelial Ovarian Cancer)

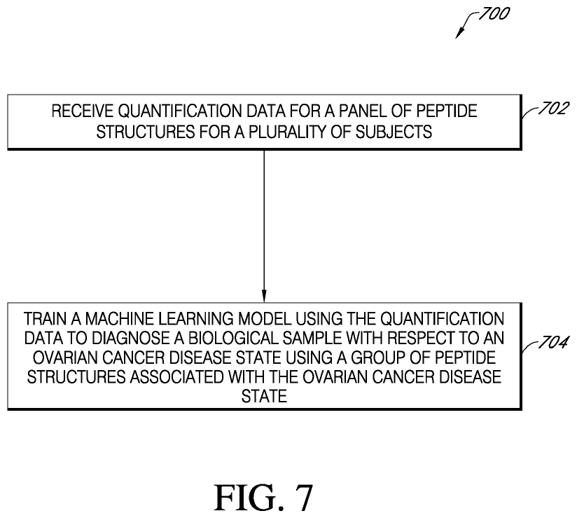

FIG. 7 is a flowchart of a process for training a model to diagnose a subject with respect to an ovarian cancer disease state in accordance with one or more embodiments. Process 700 may be implemented using, for example, at least a portion of workflow 100 as described in FIGS. 1, 2A, and 2B and/or analysis system 300 as described in FIG. 3. In some embodiments, process 700 may be one example of an implementation for training the model used in the process 500 in FIG. 5, 6, or 6B.

Step 702 includes receiving quantification data for a panel of peptide structures for a plurality of subjects. The plurality of subjects may include a first portion diagnosed with a negative diagnosis of an ovarian cancer disease state and a second portion diagnosed with a positive diagnosis of the ovarian cancer disease state. The plurality of subjects may include a first portion having early stage EOC and a second portion have late stage EOC. The quantification data comprises an initial plurality of peptide structure profiles for the plurality of subjects. For example, a peptide structure profile in the initial plurality of peptide structure profiles may include a feature associated with a corresponding peptide structure. The feature may be relative abundance, concentration, site occupancy, or some other quantification-based feature. The initial plurality of peptide structure profiles may include, one, two, three, or more profiles for a given peptide structure.

Step 704 includes training a machine learning model using the quantification data to diagnose a biological sample with respect to the ovarian cancer disease state using a group of peptide structures associated with the ovarian cancer disease state (e.g., the first group of peptide structures is identified in Table 1, the second group of peptide structures is identified in Table 2, the third group of peptide structures is identified in Table 3). The first, second, and third groups of peptide structures are listed in Tables 1, 2, and 3, respectively, with respect to relative significance to diagnosing the biological sample as evidencing malignancy (e.g., EOC). Step 704 can include training the machine learning using a portion of the quantification data corresponding to a training group of peptide structures included in the plurality of peptide structures. Step 704 can include training a machine learning model using the quantification data to assess a biological sample with respect to the staging of the ovarian cancer disease state using a group of peptide structures associated with the ovarian cancer disease state such as a group of peptide structures identified in Table 3B, 3C, or 3D.

Step 704 may include reducing the plurality of peptide structure profiles using LASSO regression to identify a final group of peptide structures identified in Table 1 above. Step 704 may include reducing the plurality of peptide structure profiles using LASSO regression to identify a final group of peptide structures identified in Table 2 above. Step 704 may include reducing the plurality of peptide structure profiles using LASSO regression to identify a final group of peptide structures identified in Tables 3B, 3C, or 3D above.

Training data can be used for training the supervised machine learning model. The training data can include a plurality of peptide structure profiles for a plurality of subjects and a plurality of subject diagnoses for the plurality of subjects. The plurality of subject diagnoses can include a positive diagnosis for any subject of the plurality of subjects determined to have the ovarian cancer disease state and a negative diagnosis for any subject of the plurality of subjects determined not to have the ovarian cancer disease state.

The machine learning model can include a binary classification model. Some binary classification models can include logistical regression models. Some logistical regression models can include LASSO regression models.

An alternative or additional step in process 700 can include filtering the initial plurality of peptide structure profiles by a coefficient of variation to generate a plurality of peptide structure profiles for use in training the machine learning model. As one example, only those peptide structure profiles having a low coefficient of variation (<20%) were included int the plurality of peptide structure profiles used for training.

An alternative or additional step in process 700 can include performing a differential expression analysis using initial training data to compare a first portion of the plurality of subjects diagnosed with the positive diagnosis for the ovarian cancer disease state versus a second portion of the plurality of subjects diagnosed with the negative diagnosis for the ovarian cancer disease state.

An alternative or additional step in process 700 can include identifying a first portion of the plurality of samples for subjects with benign pelvic tumors and malignant pelvic tumors and a second portion of the plurality of samples for subjects with a healthy status. An alternative or additional step in process 700 can include generating a training set of peptide structure profiles for 80% of the first portion and a test set of peptide structure profiles for a remaining 20% of the first portion and the second portion.

In various embodiments, the machine learning model is a supervised machine learning model that is trained to determine weight coefficients for a panel of peptide structures such that a first portion of the weight coefficients for a first portion of the panel of peptide structures are non-zero and a second portion of the weight coefficients for a second portion of the panel of peptide structures are zero (or, alternatively, substantially close to zero so as to not be statistically significant).

### VI.D. Methods of Treating Ovarian Cancer

In one or more embodiments, the final output generated in step 506 in FIG. 5 or in step 606 in FIG. 6 may include a treatment output. The treatment output may identify one or more treatment types for a subject based on the disease indicator and/or diagnosis output generated via process 500 in FIG. 5 or process 600 in FIG. 6, respectively. Treatment for ovarian cancer (e.g., EOC) may include, for example, but is not limited to, at least one of surgery, radiation therapy, a targeted drug therapy (e.g., one or more targeted therapeutic agents), chemotherapy (e.g., one or more chemotherapeutic agents), immunotherapy (e.g., one or more immunotherapeutic agents), hormone therapy, neoadjuvant therapy, or some other form of treatment. The treatment output may include, for example, a treatment plan. The treatment plan may include, for example, but is not limited to, a timeline or schedule for administering the treatment, dosing information, other treatment-related information, or a combination thereof. Being able to accurately predict malignancy via the process 500 in FIG. 5 and/or the process 600 in FIG. 6 may allow treatment for malignant pelvic tumors (e.g., EOC) to be started earlier without requiring, in many or most cases, further invasive testing such as a biopsy.

In one or more embodiments, a patient biological sample is obtained from a subject. The biological sample may be processed (e.g., via digestion and fragmentation) such that one or more peptide structures of interest are detected. For example, detection and quantification may be performed for one or more peptide structures from Table 1, Table 2, Table 3, Table 3B, Table 3C, and/or Table 3D. The quantification data that is generated for these peptide structures may be input into a trained binary classification model to generate a disease indicator, which may be, for example, a probability score. A determination may be made as to whether the disease indicator (e.g., score) is above or below a selected threshold (e.g., 0.5). If the disease indicator is above the selected threshold, the biological sample may be classified as evidencing malignant pelvic tumor.

Further, this classification may further include a classification that the subject is in need of treatment. If the subject is in need of treatment based on the classification, treatment is administered. For example, a therapeutically effective amount of a therapeutic agent is administered to the patient, where the therapeutic agent is selected from a chemotherapeutic agent, an immunotherapeutic agent, a hormone therapy, a targeted therapeutic agent, a neoadjuvant therapy, or a combination.

### VII. Peptide Structure and Product Ion Compositions, Kits and Reagents

Aspects of the disclosure include compositions comprising one or more of the peptide structures listed in Table 1, in Table 2, in Table 3, in Table 3B, in Table 3C, or in Table 3D. In some embodiments, a composition comprises a plurality of the peptide structures listed in Table 1, a plurality of the peptide structures listed in Table 2, or a plurality of the peptide structures listed in Table 3. In some embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 412, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 of the peptide structures listed in Tables 1, 2, 3, 3B, 3C, and 3D. In one or more embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or all 36 of the peptide structures listed in Table 3B. In one or more embodiments, a composition comprises 1, 2, 3, 4, 5, 6, 7, 8, 9, 10, 11, 12, 13, 14, 15, 16, 17, 18, 19, 20, 21, 22, 23, 24, or all 25 of the glycopeptide structures listed in Table 3C. In one or more embodiments, a composition comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or all 50 of the glycopeptide structures listed in Table 3D.

In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 11-19, 31-46, 53-65, 68-77, 101-125, and 126-175 listed in Tables 1, 2, 3, 3B, 3C, and 3D.

In some embodiments, a composition comprises a peptide structure having an amino acid sequence with at least 80% sequence identity, such as, for example, at least 81%, 82%, 83%, 84%, 85%, 86%, 87%, 88%, 89%, 90%, 91%, 92%, 93%, 94%, 95%, 96%, 97%, 98%, 99%, or 100% sequence identity to any one of SEQ ID NOs: 131-134, 137, 139, 140, 143, 151, 165-167 listed in Tables 3D.

Aspects of the disclosure include compositions comprising one or more precursor ions having a defined charge and/or defined mass-to-charge (m/z) ratio, as listed in Tables 4, 4B, and 4C. Aspects of the disclosure include compositions comprising one or more product ions having a defined mass-to-charge (m/z) ratio, which product ions are produced by converting a peptide structure described herein (e.g., a peptide structure listed in Tables 1, 2, 3, 3B, 3C, or 3D) into a gas phase ion in a mass spectrometry system. Conversion of the peptide structure into a gas phase ion can take place using any of a variety of techniques, including, but not limited to, matrix assisted laser desorption ionization (MALDI); electron ionization (EI); electrospray ionization (ESI); atmospheric pressure chemical ionization (APCI); and/or atmospheric pressure photo ionization (APPI).

Aspects of the disclosure include compositions comprising one or more product ions produced from one or more of the peptide structures described herein (e.g., a peptide structure listed in Tables 1, 2, 3, 3B, 3C, or 3D). In some embodiments, a composition comprises a set of the product ions listed in Table 4, 4B, or 4C having an m/z ratio selected from the list provided for each peptide structure in Table 4, 4B, or 4C.

In some embodiments, a composition comprises at least one of peptide structures PS-1 to PS-10 identified in Table 1. In some embodiments, a composition comprises at least one of peptide structures PS-11 to PS-34 and PS-5 identified in Table 2. In some embodiments, a composition comprises at least one of peptide structures PS-1, PS-5, PS-11, PS-15, PS-20, PS-25, PS-28, PS-29, PS-30, PS-31, PS-32, and PS-35 to PS-61 identified in Table 3. In some embodiments, a composition comprises at least one of peptide structures PS-4, PS-8, PS-18, PS-36, PS-37, PS-41, PS-56, PS-62 to PS-90 identified in Table 3B. In some embodiments, a composition comprises at least one of peptide structures of SEQ ID NOS 101-125 identified in Table 3C. In some embodiments, a composition comprises at least one of peptide structures of PS-ID 91 to 140 identified in Table 3D. In some embodiments, a composition comprises at least one of peptide structures of PS-ID NO: 96-99, 102, 104, 105, 108, 116, and 130-132 identified in Table 3D.

In one or more embodiments, a composition comprises at least 1, at least 2, at least 3, at least 4, at least 5, at least 6, at least 7, at least 8, at least 9, or all 10 of the peptide structures PS-1 to PS-10 identified in Table 1. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures PS-11 to PS-34 and PS-5 identified in Table 2. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, at least 36, at least 37, or all 38 of the peptide structures PS-1, PS-5, PS-11, PS-15, PS-20, PS-25, PS-28, PS-29, PS-30, PS-31, PS-32, and PS-35 to PS-61 identified in Table 3. In some embodiments, the at least 3 peptide structures additionally include at least 1, at least 2, at least 3, at least 4, at least 5, at least 6, or all 7 of the remaining peptide structures PS-1, PS-5, PS-11, PS-15, PS-20, PS-25, PS-28, PS-29, PS-30, PS-31, PS-32, and PS-35 to PS-61 identified in Table 3. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, at least 25, at least 26, at least 27, at least 28, at least 29, at least 30, at least 31, at least 32, at least 33, at least 34, at least 35, or all 36 of the peptide structures PS-4, PS-8, PS-18, PS-36, PS-37, PS-41, PS-56, PS-62 to PS-90 identified in Table 3B. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 23, at least 24, or all 25 of the peptide structures of SEQ ID NOS 121-125 identified in Table 3C. In one or more embodiments, a composition comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or all 50 of the peptide structures of SEQ ID NOS 126-175 identified in Table 3D. In one or more embodiments, a composition comprises at least 1, at least 2, at least 3, at least 4, at least 5, at least 6, at least 7, at least 8, at least 9, at least 10, at least 11, or all 12 of the peptide structures of SEQ ID NOS 131-134, 137, 139, 140, 143, 151, 165-167 identified in Table 3D.

In some embodiments, a composition comprises a peptide structure or a product ion. The peptide structure or product ion can include an amino acid sequence having at least 90% sequence identity to any one of SEQ ID NOS: 11-19, as identified in Table 5, corresponding to peptide structures PS-1 to PS-10 in Table 1. The peptide structure or product ion can include an amino acid sequence having at least 90% sequence identity to any one of SEQ ID NOS: 14, 15, 31-46, as identified in Table 5, corresponding to various ones of peptide structures PS-5 and PS-11 to PS-34 in Table 2. The peptide structure or production can include an amino acid sequence having at least 90% sequence identity to any one of SEQ ID NOS: 11, 14, 15, 31, 32, 33, 34, 37, 38, 40, 42, 44, 45, 46, 53-65, as identified in Table 5, corresponding to various ones of peptide structures PS-1, PS-5, PS-11, PS-15, PS-20, PS-25, PS-28, PS-29, PS-30, PS-31, PS-32, and PS-35 to PS-61 in Table 3. The peptide structure or product ion can include an amino acid sequence having at least 90% sequence identity to any one of SEQ ID NOS: 14, 18, 32, 33, 37, 39, 42, 45, 54, 56, 60, 68-77, as identified in Table 5, corresponding to various ones of peptide structures PS-4, PS-8, PS-18, PS-36, PS-37, PS-41, PS-56, PS-62 to PS-90 in Table 3B. The peptide structure or product ion can include an amino acid sequence having at least 90% sequence identity to any one of SEQ ID NOS: 101-125, corresponding to various ones of peptide structures in Table 3C or product ions in Table 4B. The peptide structure or product ion can include an amino acid sequence having at least 90% sequence identity to any one of SEQ ID NOS: 126-175, as identified in Table 5, corresponding to various ones of peptide structures PS-91 to PS-140 in Table 3D.

In some embodiments, the product ion is selected as one from a group consisting of product ions identified in Tables 4, 4B, and 4C including product ions falling within an identified m/z range of the m/z ratio identified in Tables 4, 4B, and 4C and characterized as having a precursor ion having an m/z ratio within an identified m/z range of the m/z ratio identified in Tables 4, 4B, and 4C. A first range for the product ion m/z ratio may be +0.5. A second range for the product ion m/z ratio may be +0.8. A third range for the product ion m/z ratio may be +1.0. A first range for the precursor ion m/z ratio may be +1.0; a second range for the precursor ion m/z ratio may be (+1.5). Thus, a composition may include a product ion having an m/z ratio that falls within at least one of the first range (+0.5), the second range (+0.8), or the third range (+1.0) of the product ion m/z ratio identified in Tables 4, 4B, and 4C, and characterized as having a precursor ion having an m/z ratio that falls within at least one of first range (±0.5), a second range (±1.0), or a third range (±1.0 of the precursor ion m/z ratio identified in Tables 4, 4B, and 4C.

TABLE 4
Mass Spectrometry-Related Characteristics for the Peptide Structures
associated with Ovarian Cancer (e.g., EOC)

| | | | | | | 2^{nd} | |
|---|---|---|---|---|---|---|---|
| | | Collision | 1^{st} | 1^{st} | 1^{st} | Collision | 2^{nd} |
| | | Energy | Precursor | Precursor | Product | Energy | Product |
| PS-ID NO. | RT (min) | (V) | m/z | Charge | m/z | (V) | m/z |
| PS-1 | 10.6 | 30 | 1115.1 | 3 | 366.1 | 34 | 1341.6 |
| PS-2 | 35.8 | 35 | 1241.8 | 4 | 204.1 | 20 | 1152.6 |
| PS-3 | 6.6 | 25 | 1009.4 | 3 | 366.1 | N/A | N/A |
| PS-4 | 17.1 | 30 | 1226.2 | 4 | 366.1 | 30 | 1048.5 |
| PS-5 | 7.9 | 21 | 879 | 3 | 204.1 | 27 | 1392.6 |
| PS-6 | 40.5 | 35 | 1184.5 | 4 | 204.1 | N/A | N/A |
| PS-7 | 33.6 | 30 | 1440.3 | 4 | 366.1 | N/A | N/A |
| PS-8 | 13.3 | 35 | 1378.9 | 5 | 366.1 | N/A | N/A |
| PS-9 | 10.1 | 35 | 1237 | 2 | 204.1 | 20 | 1376.6 |
| PS-10 | 10.1 | 35 | 1237 | 2 | 204.1 | 20 | 1376.6 |
| PS-11 | 16.3 | 20 | 891.1 | 3 | 829.4 | 20 | 366.1 |
| PS-12 | 22.6 | 31 | 1250.3 | 4 | 366.1 | N/A | N/A |
| PS-13 | 30.2 | 28 | 1173.2 | 4 | 366.1 | 978.5 | 25 |
| PS-14 | 44 | 15 | 874.4 | 5 | 366.1 | 1183.6 | 20 |
| PS-15 | 31.3 | 30 | 1191.2 | 4 | 366.1 | 978.5 | 20 |
| PS-16 | 27.4 | 35 | 1084.2 | 4 | 204.1 | N/A | N/A |
| PS-17 | 12.6 | 28 | 1106.8 | 3 | 366.1 | N/A | N/A |
| PS-18 | 37.8 | 30 | 1082.6 | 5 | 274.1 | N/A | N/A |
| PS-19 | 16.7 | 20 | 1025.7 | 4 | 274.1 | 1048.5 | 25 |
| PS-20 | 43.3 | 34 | 1341 | 5 | 366.1 | 1299 | 34 |
| PS-21 | 22.6 | 30 | 1213.8 | 4 | 366.1 | N/A | N/A |
| PS-22 | 37.3 | 30 | 1336.3 | 4 | 366.1 | N/A | N/A |
| PS-23 | 13.1 | 13 | 935.8 | 3 | 204.1 | 1360.6 | 30 |
| PS-24 | 14.8 | 25 | 1021.4 | 4 | 366.1 | N/A | N/A |
| PS-25 | 5.7 | 29 | 1165.6 | 4 | 366.1 | 979.5 | 29 |
| PS-26 | 7.9 | 30 | 1224.5 | 2 | 366.1 | N/A | N/A |
| PS-27 | 18.5 | 33 | 1055.8 | 3 | 366.1 | 1453.6 | 35 |
| PS-28 | 23.5 | 20 | 1079.7 | 4 | 366.1 | N/A | N/A |
| PS-29 | 31 | 30 | 1144.9 | 4 | 366.1 | 1359.6 | 35 |
| PS-30 | 16.5 | 34 | 1117.2 | 4 | 366.1 | N/A | N/A |
| PS-31 | 43.5 | 22 | 1057 | 4 | 366.1 | 1184.1 | 28 |
| PS-32 | 41.5 | 22 | 1115.4 | 4 | 366.1 | 366.1 | 25 |
| PS-33 | 32.3 | 30 | 1217.7 | 4 | 366.1 | 1359.6 | 35 |
| PS-34 | 13.6 | 35 | 1087.1 | 3 | 204.1 | N/A | N/A |
| PS-35 | 24.3 | 23 | 942.4 | N/A | 366.1 | N/A | N/A |
| PS-36 | 31.1 | 34 | 1343.8 | N/A | 366.1 | N/A | N/A |
| PS-37 | 23.9 | 25 | 1116.9 | N/A | 366.1 | N/A | N/A |
| PS-38 | 31.3 | 15 | 590.3 | N/A | 725.4 | N/A | N/A |
| PS-39 | 34.2 | 25 | 1149.3 | N/A | 366.1 | N/A | N/A |
| PS-40 | 28 | 27 | 1085.4 | N/A | 366.1 | N/A | N/A |
| PS-41 | 33.8 | 27 | 1105.6 | N/A | 366.1 | N/A | N/A |
| PS-42 | 31.2 | 30 | 1314.9 | N/A | 366.1 | N/A | N/A |
| PS-43 | 34.4 | 25 | 819.1 | N/A | 855.5 | N/A | N/A |
| PS-44 | 31 | 25 | 1035.6 | N/A | 366.1 | N/A | N/A |
| PS-45 | 5.6 | 25 | 1256.8 | N/A | 366.1 | N/A | N/A |
| PS-46 | 26.4 | 20 | 1252.5 | N/A | 366.1 | N/A | N/A |
| PS-47 | 31 | 33 | 1335.3 | N/A | 366.1 | N/A | N/A |
| PS-48 | 8.1 | 20 | 1054.7 | N/A | 366.1 | N/A | N/A |
| PS-49 | 40.3 | 29 | 944.5 | N/A | 1269.6 | N/A | N/A |
| PS-50 | 13.1 | 25 | 1043.8 | N/A | 366.1 | N/A | N/A |
| PS-51 | 5.8 | 34 | 1335 | N/A | 366.1 | N/A | N/A |
| PS-52 | 13.2 | 25 | 927.7 | N/A | 366.1 | N/A | N/A |
| PS-53 | 33 | 25 | 1018.1 | N/A | 366.1 | N/A | N/A |
| PS-54 | 27.4 | 25 | 1012.7 | N/A | 366.1 | N/A | N/A |
| PS-55 | 13.2 | 15 | 989.9 | N/A | 204.1 | N/A | N/A |
| PS-56 | 38.6 | 35 | 1214.1 | N/A | 274.1 | N/A | N/A |
| PS-57 | 40 | 20 | 693.9 | N/A | 675.4 | N/A | N/A |
| PS-58 | 30.4 | 26 | 1070.4 | N/A | 366.1 | N/A | N/A |
| PS-59 | 23 | 20 | 988.8 | N/A | 274.1 | N/A | N/A |
| PS-60 | 15.7 | 12 | 453.2 | N/A | 532.2 | N/A | N/A |
| PS-61 | 37.5 | 25 | 1116.9 | N/A | 366.1 | N/A | N/A |
| PS-62 | 37.9 | 30 | 1053.4 | 5 | 274.1 | N/A | N/A |
| PS-63 | 38.3 | 30 | 1184.9 | 5 | 274.1 | N/A | N/A |
| PS-64 | 30.8 | 35 | 1441.6 | 3 | 366.1 | 30 | 978.5 |
| PS-65 | 5.8 | 30 | 1213.3 | 3 | 366.1 | 30 | 979.5 |
| PS-66 | 5.7 | 32 | 1262 | 3 | 366.1 | 32 | 979.5 |
| PS-67 | 5.4 | 31 | 1238 | 3 | 366.1 | 31 | 979.5 |
| PS-68 | 5.9 | 27 | 1110.8 | 4 | 366.1 | 27 | 979.5 |
| PS-69 | 23.4 | 25 | 1195.3 | 4 | 366.1 | N/A | N/A |
| PS-70 | 23.3 | 31 | 1231.8 | 4 | 274.1 | N/A | N/A |
| PS-71 | 23.1 | 33 | 1323.1 | 4 | 366.1 | N/A | N/A |
| PS-72 | 5.4 | 35 | 1220.1 | 3 | 274.1 | 35 | 999.4 |
| PS-73 | 5.3 | 33 | 1268.8 | 3 | 274.1 | 33 | 999.4 |
| PS-74 | 37.7 | 30 | 1196.5 | 4 | 366.1 | 20 | 1062.5 |
| PS-75 | 37.5 | 25 | 1287.8 | 4 | 366.1 | 25 | 1062.5 |
| PS-76 | 4.6 | 33 | 1257.5 | 3 | 366.1 | 33 | 965.5 |
| PS-77 | 19.6 | 25 | 1028.9 | 4 | 274.1 | 25 | 1453.6 |
| PS-78 | 27.5 | 35 | 1284.8 | 4 | 204.1 | N/A | N/A |
| PS-79 | 20.8 | 30 | 1258.5 | 4 | 274.1 | 30 | 1113 |
| PS-80 | 27.7 | 29 | 1159.5 | 4 | 366.1 | 25 | 274.1 |
| PS-81 | 21.6 | 30 | 1067.7 | 4 | 366.1 | N/A | N/A |
| PS-82 | 21.5 | 25 | 1104.2 | 4 | 274.1 | N/A | N/A |
| PS-83 | 34.5 | 28 | 1138.4 | 5 | 366.1 | 35 | 1441.7 |
| PS-84 | 13.2 | 31 | 1247.7 | 5 | 366.1 | N/A | N/A |
| PS-85 | 13.4 | 34 | 1335.1 | 5 | 366.1 | N/A | N/A |
| PS-86 | 30.9 | 30 | 1201.5 | 4 | 366.1 | N/A | N/A |
| PS-87 | 30.7 | 32 | 1292.8 | 4 | 366.1 | 32 | 999.5 |
| PS-88 | 16.9 | 25 | 1106.4 | 4 | 274.1 | N/A | N/A |
| PS-89 | 23.1 | 20 | 1074.4 | 4 | 274.1 | N/A | N/A |
| PS-90 | 37.7 | 25 | 1127.9 | 5 | 366.1 | N/A | N/A |

TABLE 4B
Mass Spectrometry-Related Characteristics for the Peptide Structures
associated with Ovarian Cancer (e.g., EOC)-in accordance with Table 3C

| SEQ | | Collision | 1st | 1st | 2nd | 2nd | 1st | 2nd |
|---|---|---|---|---|---|---|---|---|
| ID | RT | Energy | Precursor | Precursor | Precursor | Precursor | Product | Product |
| NO | (min) | (V) | m/z | Charge | m/z | Charge | m/z | m/z |
| 101 | 30.6 | 30 | 1118.2 | 4 | 1118.2 | 4 | 366.1 | 978.5 |
| 102 | 5.8 | 30 | 1213.3 | 3 | 1213.3 | 3 | 366.1 | 979.5 |
| 103 | 5.7 | 32 | 1262 | 3 | 1262 | 3 | 366.1 | 979.5 |
| 104 | 5.9 | 27 | 1110.8 | 4 | 1110.8 | 4 | 366.1 | 979.5 |
| 105 | 23.3 | 31 | 1231.8 | 4 | n/a | n/a | 274.1 | n/a |
| 106 | 23.8 | 27 | 1087.7 | 5 | n/a | n/a | 366.1 | n/a |
| 107 | 23.1 | 33 | 1323.1 | 4 | n/a | n/a | 366.1 | n/a |
| 108 | 23.8 | 25 | 1116.9 | 5 | 1116.9 | 5 | 366.1 | 1060 |
| 109 | 5.4 | 35 | 1220.1 | 3 | 1220.1 | 3 | 274.1 | 999.4 |
| 110 | 37.7 | 30 | 1196.5 | 4 | 1196.5 | 4 | 366.1 | 1062.5 |
| 111 | 37.8 | 30 | 1360.5 | 4 | 1360.5 | 4 | 366.1 | 1062.5 |
| 112 | 41.3 | 32 | 1283.7 | 5 | 1283.7 | 5 | 366.1 | 1550.3 |
| 113 | 41.2 | 27 | 1313.1 | 5 | 1313.1 | 5 | 366.1 | 1550.3 |
| 114 | 13.1 | 25 | 1083.4 | 4 | n/a | n/a | 274.1 | n/a |
| 115 | 20.8 | 30 | 1258.5 | 4 | 1258.5 | 4 | 274.1 | 1113 |
| 116 | 21.6 | 30 | 1067.7 | 4 | n/a | n/a | 366.1 | n/a |
| 117 | 34.5 | 28 | 1138.4 | 5 | 1138.4 | 5 | 366.1 | 1441.7 |
| 118 | 13.4 | 34 | 1335.1 | 5 | n/a | n/a | 366.1 | n/a |
| 119 | 13.3 | 35 | 1378.9 | 5 | n/a | n/a | 366.1 | n/a |
| 120 | 31.1 | 29 | 1165 | 4 | n/a | n/a | 366.1 | n/a |
| 121 | 30.9 | 30 | 1201.5 | 4 | n/a | n/a | 366.1 | n/a |
| 122 | 30.7 | 32 | 1292.8 | 4 | 1292.8 | 4 | 366.1 | 999.5 |
| 123 | 16.9 | 25 | 1106.4 | 4 | n/a | n/a | 274.1 | n/a |
| 124 | 23.1 | 20 | 1074.4 | 4 | n/a | n/a | 274.1 | n/a |
| 125 | 33.2 | 27 | 1105.6 | 5 | 1105.6 | 5 | 366.1 | 1359.6 |

TABLE 4C
Mass Spectrometry-Related Characteristics for the Peptide Structures
associated with Ovarian Cancer (e.g., EOC)-in accordance with Table 3D

| SEQ | | Collision | 1st | 1st | 2nd | 2nd | 1st | 2nd |
|---|---|---|---|---|---|---|---|---|
| ID | RT | Energy | Precursor | Precursor | Precursor | Precursor | Product | Product |
| NO | (min) | (V) | m/z | Charge | m/z | Charge | m/z | m/z |
| 126 | 42.7 | 30 | 1282.9 | 5 | N/A | N/A | 366.1 | 1299 |
| 127 | 43.4 | 27 | 1111.7 | 4 | N/A | N/A | 366.1 | 1183.6 |
| 128 | 43.3 | 23 | 1148.3 | 4 | N/A | N/A | 366.1 | 1183.6 |
| 129 | 37.9 | 30 | 1053.4 | 5 | N/A | N/A | 274.1 | N/A |
| 130 | 38.3 | 30 | 1184.9 | 5 | N/A | N/A | 274.1 | N/A |
| 131 | 38.3 | 35 | 1214.1 | 5 | N/A | N/A | 274.1 | N/A |
| 132 | 30.8 | 35 | 1441.6 | 3 | N/A | N/A | 366.1 | 978.5 |
| 133 | 31.4 | 25 | 1154.7 | 4 | N/A | N/A | 274.1 | 978.5 |
| 134 | 5.4 | 35 | 1220.1 | 3 | N/A | N/A | 274.1 | 999.4 |
| 135 | 41 | 28 | 1152.7 | 5 | N/A | N/A | 366.1 | 1550.3 |
| 136 | 41 | 29 | 1181.9 | 5 | N/A | N/A | 366.1 | 1550.3 |
| 137 | 40.9 | 30 | 1568.4 | 4 | 1254.9 | 5 | 366.1 | 366.1 |
| 138 | 41.2 | 27 | 1313.1 | 5 | N/A | N/A | 366.1 | 1550.3 |
| 139 | 37.7 | 30 | 1196.5 | 4 | N/A | N/A | 366. | 1062.5 |
| 140 | 37.5 | 25 | 1287.8 | 4 | N/A | N/A | 366.1 | 1062.5 |
| 141 | 37.8 | 30 | 1360.5 | 4 | N/A | N/A | 366.1 | 1062.5 |
| 142 | 5.8 | 30 | 1213.3 | 3 | N/A | N/A | 366.1 | 979.5 |
| 143 | 5.7 | 32 | 1262 | 3 | N/A | N/A | 366.1 | 979.5 |
| 144 | 5.4 | 31 | 1238 | 3 | N/A | N/A | 366.1 | 979.5 |
| 145 | 5.9 | 26 | 1074.3 | 4 | N/A | N/A | 366.1 | 979.5 |
| 146 | 5.9 | 27 | 1110.8 | 4 | N/A | N/A | 366.1 | 979.5 |
| 147 | 23.4 | 25 | 1195.3 | 4 | N/A | N/A | 366.1 | N/A |
| 148 | 22.6 | 30 | 1213.8 | 4 | N/A | N/A | 366.1 | N/A |
| 149 | 23.2 | 25 | 1286.6 | 4 | N/A | N/A | 366.1 | N/A |
| 150 | 23.8 | 27 | 1087.7 | 5 | N/A | N/A | 366.1 | N/A |
| 151 | 22.6 | 31 | 1250.3 | 4 | N/A | N/A | 366.1 | N/A |
| 152 | 23.1 | 33 | 1323.1 | 4 | N/A | N/A | 366.1 | N/A |
| 153 | 23.8 | 25 | 1116.9 | 5 | N/A | N/A | 366.1 | 1060 |
| 154 | 4.8 | 30 | 1208.6 | 3 | N/A | N/A | 366.1 | 965.5 |
| 155 | 4.6 | 33 | 1257.5 | 3 | N/A | N/A | 366.1 | 965.5 |
| 156 | 4.8 | 29 | 1070.7 | 4 | N/A | N/A | 366.1 | 965.5 |
| 157 | 13.1 | 25 | 1083.4 | 4 | N/A | N/A | 274.1 | N/A |
| 158 | 19.1 | 30 | 1201.5 | 3 | N/A | N/A | 274.1 | 1453.6 |
| 159 | 27.5 | 35 | 1284.8 | 4 | N/A | N/A | 204.1 | N/A |
| 160 | 20.8 | 25 | 1222 | 4 | N/A | N/A | 274.1 | 1113 |
| 161 | 27.7 | 29 | 1159.5 | 4 | N/A | N/A | 366.1 | 274.1 |
| 162 | 27.6 | 30 | 1196 | 4 | N/A | N/A | 366.1 | 274.1 |
| 163 | 30.4 | 34 | 1343.8 | 4 | N/A | N/A | 366.1 | N/A |
| 164 | 21.6 | 30 | 1067.7 | 4 | N/A | N/A | 366.1 | N/A |
| 165 | 21.5 | 25 | 1104.2 | 4 | N/A | N/A | 274.1 | N/A |
| 166 | 34.5 | 28 | 1138.4 | 5 | N/A | N/A | 366.1 | 1441.7 |
| 167 | 13.2 | 31 | 1247.7 | 5 | N/A | N/A | 366.1 | N/A |
| 168 | 13.1 | 32 | 1276.9 | 5 | N/A | N/A | 366.1 | N/A |
| 169 | 13.4 | 34 | 1335.1 | 5 | N/A | N/A | 366.1 | N/A |
| 170 | 13.3 | 35 | 1378.9 | 5 | N/A | N/A | 366.1 | N/A |
| 171 | 28.9 | 25 | 1055.7 | 4 | N/A | N/A | 366.1 | 999.5 |
| 172 | 30 | 28 | 1128.8 | 4 | N/A | N/A | 366.1 | N/A |
| 173 | 30.9 | 30 | 1201.5 | 4 | N/A | N/A | 366.1 | N/A |
| 174 | 30.7 | 32 | 1292.8 | 4 | N/A | N/A | 366.1 | 999.5 |
| 175 | 23.1 | 20 | 1074.4 | 4 | N/A | N/A | 274.1 | N/A |

Tables 4, 4B, and 4C show various parameters associated with the identification of the peptide and glycopeptides using LC and MRM-MS. The retention time (RT) represents the amount of time in minutes for the peptide elute from the chromatography column. The collision energy represents the energy applied to the peptide for creating fragments (i.e., product ions) such as, for example, in the 2nd quadrupole of the triple quadrupole MS. The first precursor m/z represents a ratio value associated with an ionized form having a first precursor charge for the peptide or glycopeptide. Similarly, the second precursor m/z represents a ratio value associated with an ionized form having a second precursor charge for the peptide or glycopeptide. The first precursor ion is associated with a first product ion having a m/z ratio that was formed from a collision and the second precursor ion is associated with a second product ion having a m/z ratio that was formed from a collision. Under certain circumstances, the first precursor and the second precursor may be the same, but the associated first and second product m/z ratios are different.

Table 5 defines the peptide sequences for SEQ ID NOS: 11-19, 31-46, 53-65, 68-77, and 126-175 from at least one of Tables 1, 2, 3, 3B, 3C, and 3D. Table 5 further identifies a corresponding protein SEQ ID NO. for each peptide sequence.

TABLE 5
Peptide SEQ ID NOS

| | | Corre- |
|---|---|---|
| | | sponding |
| SEQ | | Protein |
| ID | | SEQ ID |
| NO: | Peptide Sequence | NO: |
| 11 | FGCEIENNR | 1 |
| 12 | VLSNNSDANLELINTWVAK | 2 |
| 13 | LISNCSK | 3 |
| 14 | EHEGAIYPDNTTDFQR | 4 |
| 15 | EEQYNSTYR | 5 |
| 16 | CSDGWSFDATTLDDNGTMLFFK | 6 |
| 17 | QVFPGLNYCTSGAYSNASSTDSASYYPLTGDTR | 7 |
| 18 | NLFLNHSENATAK | 8 |
| 19 | EEQYNSTFR | 9,10 |
| 31 | QSVPAHFVALNGSK | 20 |
| 32 | QDQCIYNTTYLNVQR | 21 |
| 33 | YTGNASALFILPDQDK | 22 |
| 34 | VSNQTLSLFFTVLQDVPVR | 23 |
| 35 | ENLTAPGSDSAVFFEQGTTR | 4 |
| 36 | FVEGSHNSTVSLTTK | 7 |
| 37 | FNLTETSEAEIHQSFQHLLR | 22 |
| 38 | ADTHDEILEGLNFNLTEIPEAQIHEGFQELLR | 24 |
| 39 | NISDGFDGIPDNVDAALALPAHSYSGR | 25 |
| 40 | EEQFNSTFR | 26 |
| 41 | IPCSQPPQIEHGTINSSR | 27 |
| 42 | ENGTISR | 21 |
| 43 | LGNWSAMPSCK | 28 |
| 44 | ADGTVNQIEGEATPVNLTEPAK | 29 |
| 45 | QQQHLFGSNVTDCSGNFCLFR | 30 |
| 46 | GCVLLSYLNETVTVSASLESVR | 23 |
| 53 | NGSLFAFR | 25 |
| 54 | AALAAFNAQNNGSNFQLEEISR | 47 |
| 55 | DLLLPQPDLR | 48 |
| 56 | MVSHHNLTTGATLINEQWLLTTAK | 8 |
| 57 | CGLVPVLAENYNK | 30 |
| 58 | ALPQPQNVTSLLGCTH | 6 |
| 59 | TSESGELHGLTTEEEFVEGIYK | 49 |
| 60 | VVLHPNYSQVDIGLIK | 8 |
| 61 | SDVGFLPPFPTLDPEEK | 50 |
| 62 | VSFLSALEEYTK | 51 |
| 63 | TVVQPSVGAAAGPVVPPCPGR | 47 |
| 64 | THLAPYSDELR | 51 |
| 65 | FSLLGHASISCTVENETIGVWRPSPPTCEK | 52 |
| 68 | NEEYNK | 21, 66 |
| 69 | SVQEIQATFFYFTPNK | 21, 66 |
| 70 | ENGTVSR | 66 |
| 71 | LGNWSAMPSCK | 28 |
| 72 | ENLTAPGSDSAVFFEQGTTR | 4 |
| 73 | ELHHLQEQNVSNAFLDK | 4 |
| 74 | VCQDCPLLAPLNDTR | 47 |
| 75 | SWPAVGNCSSALR | 6 |
| 75 | SWPAVGNCSSALR | 6 |
| 76 | ITYSIVQTNCSK | 67 |
| 77 | LNAENNATFYFK | 67 |
| 126 | ADTHDEILEGLNFNLTEIPEAQIHEGFQELLR | 24 |
| 127 | VSNQTLSLFFTVLQDVPVR | 23 |
| 128 | VSNQTLSLFFTVLQDVPVR | 23 |
| 129 | FNLTETSEAEIHQSFQHLLR | 22 |
| 130 | FNLTETSEAEIHQSFQHLLR | 22 |
| 131 | FNLTETSEAEIHQSFQHLLR | 22 |
| 132 | YTGNASALFILPDQDK | 22 |
| 133 | YTGNASALFILPDQDK | 22 |
| 134 | NEEYNK | 21 or 66 |
| 135 | SVQEIQATFFYFTPNKTEDTIFLR | 22 or 66 |
| 136 | SVQEIQATFFYFTPNKTEDTIFLR | 23 or 66 |
| 137 | SVQEIQATFFYFTPNKTEDTIFLR | 24 or 66 |
| 138 | SVQEIQATFFYFTPNKTEDTIFLR | 25 or 66 |
| 139 | SVQEIQATFFYFTPNK | 26 or 66 |
| 140 | SVQEIQATFFYFTPNK | 27 or 66 |
| 141 | SVQEIQATFFYFTPNK | 28 or 66 |
| 142 | ENGTISR | 21 |
| 143 | ENGTISR | 21 |
| 144 | ENGTISR | 21 |
| 145 | ENGTISR | 21 |
| 146 | ENGTISR | 21 |
| 147 | QDQCIYNTTYLNVQR | 21 |
| 148 | QDQCIYNTTYLNVQR | 21 |
| 149 | QDQCIYNTTYLNVQR | 21 |
| 150 | QDQCIYNTTYLNVQR | 21 |
| 151 | QDQCIYNTTYLNVQR | 21 |
| 152 | QDQCIYNTTYLNVQR | 21 |
| 153 | QDQCIYNTTYLNVQR | 21 |
| 154 | ENGTVSR | 66 |
| 155 | ENGTVSR | 66 |
| 156 | ENGTVSR | 66 |
| 157 | VYKPSAGNNSLYR | 28 |
| 158 | LGNWSAMPSCK | 28 |
| 159 | ENLTAPGSDSAVFFEQGTTR | 4 |
| 160 | ELHHLQEQNVSNAFLDK | 4 |
| 161 | VCQDCPLLAPLNDTR | 47 |
| 162 | VCQDCPLLAPLNDTR | 47 |
| 163 | AALAAFNAQNNGSNFQLEEISR | 47 |
| 164 | SWPAVGNCSSALR | 6 |
| 165 | SWPAVGNCSSALR | 6 |
| 166 | MVSHHNLTTGATLINEQWLLTTAK | 8 |
| 167 | NLFLNHSENATAK | 8 |
| 168 | NLFLNHSENATAK | 8 |
| 169 | NLFLNHSENATAK | 8 |
| 170 | NLFLNHSENATAK | 8 |
| 171 | VVLHPNYSQVDIGLIK | 8 |
| 172 | VVLHPNYSQVDIGLIK | 8 |
| 173 | VVLHPNYSQVDIGLIK | 8 |
| 174 | VVLHPNYSQVDIGLIK | 8 |
| 175 | LNAENNATFYFK | 67 |

Table 6 identifies the proteins of SEQ ID NOS: 1-10, 20-30, 47-52, and 66-67 from at least of one of Tables 1, 2, 3, 3B, 3C, and 3D. Table 6 identifies a corresponding protein abbreviation and protein name for each of protein SEQ ID NOS: 1-10, 20-30, 47-52, and 66-67. Further, Table 6 identifies a corresponding Uniprot ID and protein sequence for each of protein SEQ ID NOS: 1-10, 20-30, 47-52, and 66-67.

TABLE 6
Protein SEQ ID NOS

| SEQ | | | | |
|---|---|---|---|---|
| ID | Protein | | Uniprot | |
| NO. | Abbreviation | Protein Name | ID | Protein Sequence |
| 1 | ZA2G | Zinc-alpha-2- | P25311 | MVRMVPVLLSLLLLLGPAVPQENQDGR |
| | | glycoprotein | | YSLTYIYTGLSKHVEDVPAFQALGSLN |
| | | | | DLQFFRYNSKDRKSQPMGLWRQVEGM |
| | | | | EDWKQDSQLQKAREDIFMETLKDIVEY |
| | | | | YNDSNGSHVLQGRFGCEIENNRSSGAF |
| | | | | WKYYYDGKDYIEFNKEIPAWVPFDPAA |
| | | | | QITKQKWEAEPVYVQRAKAYLEEECPA |
| | | | | TLRKYLKYSKNILDRQDPPSVVVTSHQ |
| | | | | APGEKKKLKCLAYDFYPGKIDVHWTR |
| | | | | AGEVQEPELRGDVLHNGNGTYQSWVV |
| | | | | VAVPPQDTAPYSCHVQHSSLAQPLVVP |
| | | | | WEAS |
| 2 | IC1 | Plasma | P05155 | MASRLTLLTLLLLLLAGDRASSNPNATS |
| | | protease C1 | | SSSQDPESLQDRGEGKVATTVISKMLFV |
| | | inhibitor | | EPILEVSSLPTTNSTTNSATKITANTTDE |
| | | | | PTTQPTTEPTTQPTIQPTQPTTQLPTDSP |
| | | | | TQPTTGSFCPGPVTLCSDLESHSTEAVL |
| | | | | GDALVDFSLKLYHAFSAMKKVETNMA |
| | | | | FSPFSIASLLTQVLLGAGENTKTNLESIL |
| | | | | SYPKDFTCVHQALKGFTTKGVTSVSQIF |
| | | | | HSPDLAIRDTFVNASRTLYSSSPRVLSN |
| | | | | NSDANLELINTWVAKNTNNKISRLLDS |
| | | | | LPSDTRLVLLNAIYLSAKWKTTFDPKKT |
| | | | | RMEPFHFKNSVIKVPMMNSKKYPVAHF |
| | | | | IDQTLKAKVGQLQLSHNLSLVILVPQNL |
| | | | | KHRLEDMEQALSPSVFKAIMEKLEMSK |
| | | | | FQPTLLTLPRIKVTTSQDMLSIMEKLEFF |
| | | | | DFSYDLNLCGLTEDPDLQVSAMQHQTV |
| | | | | LELTETGVEAAAASAISVARTLLVFEVQ |
| | | | | QPFLFVLWDQQHKFPVFMGRVYDPRA |
| 3 | CFAI | Complement | P05156 | MKLLHVFLLFLCFHLRFCKVTYTSQED |
| | | Factor I | | LVEKKCLAKKYTHLSCDKVFCQPWQR |
| | | | | CIEGTCVCKLPYQCPKNGTAVCATNRR |
| | | | | SFPTYCQQKSLECLHPGTKFLNNGTCTA |
| | | | | EGKFSVSLKHGNTDSEGIVEVKLVDQD |
| | | | | KTMFICKSSWSMREANVACLDLGFQQ |
| | | | | GADTQRRFKLSDLSINSTECLHVHCRGL |
| | | | | ETSLAECTFTKRRTMGYQDFADVVCYT |
| | | | | QKADSPMDDFFQCVNGKYISQMKACD |
| | | | | GINDCGDQSDELCCKACQGKGFHCKSG |
| | | | | VCIPSQYQCNGEVDCITGEDEVGCAGF |
| | | | | ASVTQEETEILTADMDAERRRIKSLLPK |
| | | | | LSCGVKNRMHIRRKRIVGGKRAQLGDL |
| | | | | PWQVAIKDASGITCGGIYIGGCWILTAA |
| | | | | HCLRASKTHRYQIWTTVVDWIHPDLKR |
| | | | | IVIEYVDRIIFHENYNAGTYQNDIALIEM |
| | | | | KKDGNKKDCELPRSIPACVPWSPYLFQ |
| | | | | PNDTCIVSGWGREKDNERVFSLQWGEV |
| | | | | KLISNCSKFYGNRFYEKEMECAGTYDG |
| | | | | SIDACKGDSGGPLVCMDANNVTYVWG |
| | | | | VVSWGENCGKPEFPGVYTKVANYFDW |
| | | | | ISYHVGRPFISQYNV |
| 4 | CERU | Ceruloplasmin | P00450 | MKILILGIFLFLCSTPAWAKEKHYYIGII |
| | | | | ETTWDYASDHGEKKLISVDTEHSNIYL |
| | | | | QNGPDRIGRLYKKALYLQYTDETFRTTI |
| | | | | EKPVWLGFLGPIIKAETGDKVYVHLKN |
| | | | | LASRPYTFHSHGITYYKEHEGAIYPDNT |
| | | | | TDFQRADDKVYPGEQYTYMLLATEEQ |
| | | | | SPGEGDGNCVTRIYHSHIDAPKDIASGLI |
| | | | | GPLIICKKDSLDKEKEKHIDREFVVMFS |
| | | | | VVDENFSWYLEDNIKTYCSEPEKVDKD |
| | | | | NEDFQESNRMYSVNGYTFGSLPGLSMC |
| | | | | AEDRVKWYLFGMGNEVDVHAAFFHG |
| | | | | QALTNKNYRIDTINLFPATLFDAYMVA |
| | | | | QNPGEWMLSCQNLNHLKAGLQAFFQV |
| | | | | QECNKSSSKDNIRGKHVRHYYIAAEEII |
| | | | | WNYAPSGIDIFTKENLTAPGSDSAVFFE |
| | | | | QGTTRIGGSYKKLVYREYTDASFTNRK |
| | | | | ERGPEEEHLGILGPVIWAEVGDTIRVTF |
| | | | | HNKGAYPLSIEPIGVRFNKNNEGTYYSP |
| | | | | NYNPQSRSVPPSASHVAPTETFTYEWT |
| | | | | VPKEVGPTNADPVCLAKMYYSAVDPT |
| | | | | KDIFTGLIGPMKICKKGSLHANGRQKD |
| | | | | VDKEFYLFPTVFDENESLLLEDNIRMFT |
| | | | | TAPDQVDKEDEDFQESNKMHSMNGFM |
| | | | | YGNQPGLTMCKGDSVVWYLFSAGNEA |
| | | | | DVHGIYFSGNTYLWRGERRDTANLFPQ |
| | | | | TSLTLHMWPDTEGTFNVECLTTDHYTG |
| | | | | GMKQKYTVNQCRRQSEDSTFYLGERT |
| | | | | YYIAAVEVEWDYSPQREWEKELHHLQ |
| | | | | EQNVSNAFLDKGEFYIGSKYKKVVYRQ |
| | | | | YTDSTFRVPVERKAEEEHLGILGPQLHA |
| | | | | DVGDKVKIIFKNMATRPYSIHAHGVQT |
| | | | | ESSTVTPTLPGETLTYVWKIPERSGAGT |
| | | | | EDSACIPWAYYSTVDQVKDLYSGLIGP |
| | | | | LIVCRRPYLKVFNPRRKLEFALLFLVFD |
| | | | | ENESWYLDDNIKTYSDHPEKVNKDDEE |
| | | | | FIESNKMHAINGRMFGNLQGLTMHVG |
| | | | | DEVNWYLMGMGNEIDLHTVHFHGHSF |
| | | | | QYKHRGVYSSDVFDIFPGTYQTLEMFP |
| | | | | RTPGIWLLHCHVTDHIHAGMETTYTVL |
| | | | | QNEDTKSG |
| 5 | IGG1 | Immunoglobulin | P01857 | ASTKGPSVFPLAPSSKSTSGGTAALGCL |
| | | heavy | | VKDYFPEPVTVSWNSGALTSGVHTFPA |
| | | constant | | VLQSSGLYSLSSVVTVPSSSLGTQTYIC |
| | | gamma 1 | | NVNHKPSNTKVDKKVEPKSCDKTHTCP |
| | | | | PCPAPELLGGPSVFLFPPKPKDTLMISRT |
| | | | | PEVTCVVVDVSHEDPEVKFNWYVDGV |
| | | | | EVHNAKTKPREEQYNSTYRVVSVLTVL |
| | | | | HQDWLNGKEYKCKVSNKALPAPIEKTI |
| | | | | SKAKGQPREPQVYTLPPSRDELTKNQV |
| | | | | SLTCLVKGFYPSDIAVEWESNGQPENN |
| | | | | YKTTPPVLDSDGSFFLYSKLTVDKSRW |
| | | | | QQGNVFSCSVMHEALHNHYTQKSLSLS |
| | | | | PGK |
| 6 | HEMO | Hemopexin | P02790 | MARVLGAPVALGLWSLCWSLAIATPLP |
| | | | | PTSAHGNVAEGETKPDPDVTERCSDGW |
| | | | | SFDATTLDDNGTMLFFKGEFVWKSHK |
| | | | | WDRELISERWKNFPSPVDAAFRQGHNS |
| | | | | VFLIKGDKVWVYPPEKKEKGYPKLLQD |
| | | | | EFPGIPSPLDAAVECHRGECQAEGVLFF |
| | | | | QGDREWFWDLATGTMKERSWPAVGN |
| | | | | CSSALRWLGRYYCFQGNQFLRFDPVRG |
| | | | | EVPPRYPRDVRDYFMPCPGRGHGHRN |
| | | | | GTGHGNSTHHGPEYMRCSPHLVLSALT |
| | | | | SDNHGATYAFSGTHYWRLDTSRDGWH |
| | | | | SWPIAHQWPQGPSAVDAAFSWEEKLYL |
| | | | | VQGTQVYVFLTKGGYTLVSGYPKRLEK |
| | | | | EVGTPHGIILDSVDAAFICPGSSRLHIMA |
| | | | | GRRLWWLDLKSGAQATWTELPWPHEK |
| | | | | VDGALCMEKSLGPNSCSANGPGLYLIH |
| | | | | GPNLYCYSDVEKLNAAKALPQPQNVTS |
| | | | | LLGCTH |
| 7 | APOB | Apolipoprotein | P04114 | MDPPRPALLALLALPALLLLLLAGARA |
| | | B-100 | | EEEMLENVSLVCPKDATRFKHLRKYTY |
| | | | | NYEAESSSGVPGTADSRSATRINCKVEL |
| | | | | EVPQLCSFILKTSQCTLKEVYGFNPEGK |
| | | | | ALLKKTKNSEEFAAAMSRYELKLAIPE |
| | | | | GKQVFLYPEKDEPTYILNIKRGIISALLV |
| | | | | PPETEEAKQVLFLDTVYGNCSTHFTVK |
| | | | | TRKGNVATEISTERDLGQCDRFKPIRTG |
| | | | | ISPLALIKGMTRPLSTLISSSQSCQYTLD |
| | | | | AKRKHVAEAICKEQHLFLPFSYKNKYG |
| | | | | MVAQVTQTLKLEDTPKINSRFFGEGTK |
| | | | | KMGLAFESTKSTSPPKQAEAVLKTLQE |
| | | | | LKKLTISEQNIQRANLFNKLVTELRGLS |
| | | | | DEAVTSLLPQLIEVSSPITLQALVQCGQP |
| | | | | QCSTHILQWLKRVHANPLLIDVVTYLV |
| | | | | ALIPEPSAQQLREIFNMARDQRSRATLY |
| | | | | ALSHAVNNYHKTNPTGTQELLDIANYL |
| | | | | MEQIQDDCTGDEDYTYLILRVIGNMGQ |
| | | | | TMEQLTPELKSSILKCVQSTKPSLMIQK |
| | | | | AAIQALRKMEPKDKDQEVLLQTFLDDA |
| | | | | SPGDKRLAAYLMLMRSPSQADINKIVQI |
| | | | | LPWEQNEQVKNFVASHIANILNSEELDI |
| | | | | QDLKKLVKEALKESQLPTVMDFRKFSR |
| | | | | NYQLYKSVSLPSLDPASAKIEGNLIFDP |
| | | | | NNYLPKESMLKTTLTAFGFASADLIEIG |
| | | | | LEGKGFEPTLEALFGKQGFFPDSVNKAL |
| | | | | YWVNGQVPDGVSKVLVDHFGYTKDD |
| | | | | KHEQDMVNGIMLSVEKLIKDLKSKEVP |
| | | | | EARAYLRILGEELGFASLHDLQLLGKLL |
| | | | | LMGARTLQGIPQMIGEVIRKGSKNDFFL |
| | | | | HYIFMENAFELPTGAGLQLQISSSGVIAP |
| | | | | GAKAGVKLEVANMQAELVAKPSVSVE |
| | | | | FVTNMGIIIPDFARSGVQMNTNFFHESG |
| | | | | LEAHVALKAGKLKFIIPSPKRPVKLLSG |
| | | | | GNTLHLVSTTKTEVIPPLIENRQSWSVC |
| | | | | KQVFPGLNYCTSGAYSNASSTDSASYY |
| | | | | PLTGDTRLELELRPTGEIEQYSVSATYE |
| | | | | LQREDRALVDTLKFVTQAEGAKQTEAT |
| | | | | MTFKYNRQSMTLSSEVQIPDFDVDLGTI |
| | | | | LRVNDESTEGKTSYRLTLDIQNKKITEV |
| | | | | ALMGHLSCDTKEERKIKGVISIPRLQAE |
| | | | | ARSEILAHWSPAKLLLQMDSSATAYGS |
| | | | | TVSKRVAWHYDEEKIEFEWNTGTNVD |
| | | | | TKKMTSNFPVDLSDYPKSLHMYANRLL |
| | | | | DHRVPQTDMTFRHVGSKLIVAMSSWL |
| | | | | QKASGSLPYTQTLQDHLNSLKEFNLQN |
| | | | | MGLPDFHIPENLFLKSDGRVKYTLNKN |
| | | | | SLKIEIPLPFGGKSSRDLKMLETVRTPAL |
| | | | | HFKSVGFHLPSREFQVPTFTIPKLYQLQ |
| | | | | VPLLGVLDLSTNVYSNLYNWSASYSGG |
| | | | | NTSTDHFSLRARYHMKADSVVDLLSYN |
| | | | | VQGSGETTYDHKNTFTLSYDGSLRHKF |
| | | | | LDSNIKFSHVEKLGNNPVSKGLLIFDAS |
| | | | | SSWGPQMSASVHLDSKKKQHLFVKEV |
| | | | | KIDGQFRVSSFYAKGTYGLSCQRDPNT |
| | | | | GRLNGESNLRFNSSYLQGTNQITGRYE |
| | | | | DGTLSLTSTSDLQSGIIKNTASLKYENY |
| | | | | ELTLKSDTNGKYKNFATSNKMDMTFS |
| | | | | KQNALLRSEYQADYESLRFFSLLSGSLN |
| | | | | SHGLELNADILGTDKINSGAHKATLRIG |
| | | | | QDGISTSATTNLKCSLLVLENELNAELG |
| | | | | LSGASMKLTTNGRFREHNAKFSLDGKA |
| | | | | ALTELSLGSAYQAMILGVDSKNIFNFKV |
| | | | | SQEGLKLSNDMMGSYAEMKFDHTNSL |
| | | | | NIAGLSLDFSSKLDNIYSSDKFYKQTVN |
| | | | | LQLQPYSLVTTLNSDLKYNALDLTNNG |
| | | | | KLRLEPLKLHVAGNLKGAYQNNEIKHI |
| | | | | YAISSAALSASYKADTVAKVQGVEFSH |
| | | | | RLNTDIAGLASAIDMSTNYNSDSLHFSN |
| | | | | VFRSVMAPFTMTIDAHTNGNGKLALW |
| | | | | GEHTGQLYSKFLLKAEPLAFTFSHDYK |
| | | | | GSTSHHLVSRKSISAALEHKVSALLTPA |
| | | | | EQTGTWKLKTQFNNNEYSQDLDAYNT |
| | | | | KDKIGVELTGRTLADLTLLDSPIKVPLL |
| | | | | LSEPINIIDALEMRDAVEKPQEFTIVAFV |
| | | | | KYDKNQDVHSINLPFFETLQEYFERNR |
| | | | | QTIIVVLENVQRNLKHINIDQFVRKYRA |
| | | | | ALGKLPQQANDYLNSFNWERQVSHAK |
| | | | | EKLTALTKKYRITENDIQIALDDAKINF |
| | | | | NEKLSQLQTYMIQFDQYIKDSYDLHDL |
| | | | | KIAIANIIDEIIEKLKSLDEHYHIRVNLVK |
| | | | | TIHDLHLFIENIDENKSGSSTASWIQNVD |
| | | | | TKYQIRIQIQEKLQQLKRHIQNIDIQHLA |
| | | | | GKLKQHIEAIDVRVLLDQLGTTISFERIN |
| | | | | DILEHVKHFVINLIGDFEVAEKINAFRA |
| | | | | KVHELIERYEVDQQIQVLMDKLVELAH |
| | | | | QYKLKETIQKLSNVLQQVKIKDYFEKL |
| | | | | VGFIDDAVKKLNELSFKTFIEDVNKFLD |
| | | | | MLIKKLKSFDYHQFVDETNDKIREVTQ |
| | | | | RLNGEIQALELPQKAEALKLFLEETKAT |
| | | | | VAVYLESLQDTKITLIINWLQEALSSAS |
| | | | | LAHMKAKFRETLEDTRDRMYQMDIQQ |
| | | | | ELQRYLSLVGQVYSTLVTYISDWWTLA |
| | | | | AKNLTDFAEQYSIQDWAKRMKALVEQ |
| | | | | GFTVPEIKTILGTMPAFEVSLQALQKAT |
| | | | | FQTPDFIVPLTDLRIPSVQINFKDLKNIKI |
| | | | | PSRFSTPEFTILNTFHIPSFTIDFVEMKVK |
| | | | | IIRTIDQMLNSELQWPVPDIYLRDLKVE |
| | | | | DIPLARITLPDFRLPEIAIPEFIIPTLNLND |
| | | | | FQVPDLHIPEFQLPHISHTIEVPTFGKLY |
| | | | | SILKIQSPLFTLDANADIGNGTTSANEAG |
| | | | | IAASITAKGESKLEVLNFDFQANAQLSN |
| | | | | PKINPLALKESVKFSSKYLRTEHGSEML |
| | | | | FFGNAIEGKSNTVASLHTEKNTLELSNG |
| | | | | VIVKINNQLTLDSNTKYFHKLNIPKLDF |
| | | | | SSQADLRNEIKTLLKAGHIAWTSSGKGS |
| | | | | WKWACPRFSDEGTHESQISFTIEGPLTS |
| | | | | FGLSNKINSKHLRVNQNLVYESGSLNFS |
| | | | | KLEIQSQVDSQHVGHSVLTAKGMALFG |
| | | | | EGKAEFTGRHDAHLNGKVIGTLKNSLF |
| | | | | FSAQPFEITASTNNEGNLKVRFPLRLTG |
| | | | | KIDFLNNYALFLSPSAQQASWQVSARF |
| | | | | NQYKYNQNFSAGNNENIMEAHVGING |
| | | | | EANLDFLNIPLTIPEMRLPYTIITTPPLKD |
| | | | | FSLWEKTGLKEFLKTTKQSFDLSVKAQ |
| | | | | YKKNKHRHSITNPLAVLCEFISQSIKSFD |
| | | | | RHFEKNRNNALDFVTKSYNETKIKFDK |
| | | | | YKAEKSHDELPRTFQIPGYTVPVVNVE |
| | | | | VSPFTIEMSAFGYVFPKAVSMPSFSILGS |
| | | | | DVRVPSYTLILPSLELPVLHVPRNLKLSL |
| | | | | PDFKELCTISHIFIPAMGNITYDFSFKSSV |
| | | | | ITLNTNAELFNQSDIVAHLLSSSSSVIDA |
| | | | | LQYKLEGTTRLTRKRGLKLATALSLSN |
| | | | | KFVEGSHNSTVSLTTKNMEVSVATTTK |
| | | | | AQIPILRMNFKQELNGNTKSKPTVSSSM |
| | | | | EFKYDFNSSMLYSTAKGAVDHKLSLES |
| | | | | LTSYFSIESSTKGDVKGSVLSREYSGTIA |
| | | | | SEANTYLNSKSTRSSVKLQGTSKIDDIW |
| | | | | NLEVKENFAGEATLQRIYSLWEHSTKN |
| | | | | HLQLEGLFFTNGEHTSKATLELSPWQM |
| | | | | SALVQVHASQPSSFHDFPDLGQEVALN |
| | | | | ANTKNQKIRWKNEVRIHSGSFQSQVEL |
| | | | | SNDQEKAHLDIAGSLEGHLRFLKNIILP |
| | | | | VYDKSLWDFLKLDVTTSIGRRQHLRVS |
| | | | | TAFVYTKNPNGYSFSIPVKVLADKFIIPG |
| | | | | LKLNDLNSVLVMPTFHVPFTDLQVPSC |
| | | | | KLDFREIQIYKKLRTSSFALNLPTLPEVK |
| | | | | FPEVDVLTKYSQPEDSLIPFFEITVPESQ |
| | | | | LTVSQFTLPKSVSDGIAALDLNAVANKI |
| | | | | ADFELPTIIVPEQTIEIPSIKFSVPAGIVIPS |
| | | | | FQALTARFEVDSPVYNATWSASLKNKA |
| | | | | DYVETVLDSTCSSTVQFLEYELNVLGT |
| | | | | HKIEDGTLASKTKGTFAHRDFSAEYEE |
| | | | | DGKYEGLQEWEGKAHLNIKSPAFTDLH |
| | | | | LRYQKDKKGISTSAASPAVGTVGMDM |
| | | | | DEDDDFSKWNFYYSPQSSPDKKLTIFKT |
| | | | | ELRVRESDEETQIKVNWEEEAASGLLTS |
| | | | | LKDNVPKATGVLYDYVNKYHWEHTGL |
| | | | | TLREVSSKLRRNLQNNAEWVYQGAIRQ |
| | | | | IDDIDVRFQKAASGTTGTYQEWKDKAQ |
| | | | | NLYQELLTQEGQASFQGLKDNVFDGLV |
| | | | | RVTQEFHMKVKHLIDSLIDFLNFPRFQF |
| | | | | PGKPGIYTREELCTMFIREVGTVLSQVY |
| | | | | SKVHNGSEILFSYFQDLVITLPFELRKHK |
| | | | | LIDVISMYRELLKDLSKEAQEVFKAIQS |
| | | | | LKTTEVLRNLQDLLQFIFQLIEDNIKQLK |
| | | | | EMKFTYLINYIQDEINTIFSDYIPYVFKL |
| | | | | LKENLCLNLHKFNEFIQNELQEASQELQ |
| | | | | QIHQYIMALREEYFDPSIVGWTVKYYE |
| | | | | LEEKIVSLIKNLLVALKDFHSEYIVSASN |
| | | | | FTSQLSSQVEQFLHRNIQEYLSILTDPDG |
| | | | | KGKEKIAELSATAQEIIKSQAIATKKIISD |
| | | | | YHQQFRYKLQDFSDQLSDYYEKFIAES |
| | | | | KRLIDLSIQNYHTFLIYITELLKKLQSTT |
| | | | | VMNPYMKLAPGELTIIL |
| 8 | HPT | Haptoglobin | P00738 | MSALGAVIALLLWGQLFAVDSGNDVT |
| | | | | DIADDGCPKPPEIAHGYVEHSVRYQCK |
| | | | | NYYKLRTEGDGVYTLNDKKQWINKAV |
| | | | | GDKLPECEADDGCPKPPEIAHGYVEHS |
| | | | | VRYQCKNYYKLRTEGDGVYTLNNEKQ |
| | | | | WINKAVGDKLPECEAVCGKPKNPANP |
| | | | | VQRILGGHLDAKGSFPWQAKMVSHHN |
| | | | | LTTGATLINEQWLLTTAKNLFLNHSEN |
| | | | | ATAKDIAPTLTLYVGKKQLVEIEKVVL |
| | | | | HPNYSQVDIGLIKLKQKVSVNERVMPIC |
| | | | | LPSKDYAEVGRVGYVSGWGRNANFKF |
| | | | | TDHLKYVMLPVADQDQCIRHYEGSTVP |
| | | | | EKKTPKSPVGVQPILNEHTFCAGMSKY |
| | | | | QEDTCYGDAGSAFAVHDLEEDTWYAT |
| | | | | GILSFDKSCAVAEYGVYVKVTSIQDWV |
| | | | | QKTIAEN |
| 9 | IGG3 | Immunoglobulin | P01860 | ASTKGPSVFPLAPCSRSTSGGTAALGCL |
| | | heavy | | VKDYFPEPVTVSWNSGALTSGVHTFPA |
| | | constant | | VLQSSGLYSLSSVVTVPSSSLGTQTYTC |
| | | gamma 3 | | NVNHKPSNTKVDKRVELKTPLGDTTHT |
| | | | | CPRCPEPKSCDTPPPCPRCPEPKSCDTPP |
| | | | | PCPRCPEPKSCDTPPPCPRCPAPELLGGP |
| | | | | SVFLFPPKPKDTLMISRTPEVTCVVVDV |
| | | | | SHEDPEVQFKWYVDGVEVHNAKTKPR |
| | | | | EEQYNSTFRVVSVLTVLHQDWLNGKE |
| | | | | YKCKVSNKALPAPIEKTISKTKGQPREP |
| | | | | QVYTLPPSREEMTKNQVSLTCLVKGFY |
| | | | | PSDIAVEWESSGQPENNYNTTPPMLDS |
| | | | | DGSFFLYSKLTVDKSRWQQGNIFSCSV |
| | | | | MHEALHNRFTQKSLSLSPGK |
| 10 | IGG4 | Immunoglobulin | P01861 | ASTKGPSVFPLAPCSRSTSESTAALGCL |
| | | heavy | | VKDYFPEPVTVSWNSGALTSGVHTFPA |
| | | constant | | VLQSSGLYSLSSVVTVPSSSLGTKTYTC |
| | | gamma 3 | | NVDHKPSNTKVDKRVESKYGPPCPSCP |
| | | | | APEFLGGPSVFLFPPKPKDTLMISRTPEV |
| | | | | TCVVVDVSQEDPEVQFNWYVDGVEVH |
| | | | | NAKTKPREEQFNSTYRVVSVLTVLHQD |
| | | | | WLNGKEYKCKVSNKGLPSSIEKTISKA |
| | | | | KGQPREPQVYTLPPSQEEMTKNQVSLT |
| | | | | CLVKGFYPSDIAVEWESNGQPENNYKT |
| | | | | TPPVLDSDGSFFLYSRLTVDKSRWQEG |
| | | | | NVFSCSVMHEALHNHYTQKSLSLSLGK |
| 20 | CO2 | ComplementC2 | P06681 | MGPLMVLFCLLFLYPGLADSAPSCPQN |
| | | | | VNISGGTFTLSHGWAPGSLLTYSCPQGL |
| | | | | YPSPASRLCKSSGQWQTPGATRSLSKA |
| | | | | VCKPVRCPAPVSFENGIYTPRLGSYPVG |
| | | | | GNVSFECEDGFILRGSPVRQCRPNGMW |
| | | | | DGETAVCDNGAGHCPNPGISLGAVRTG |
| | | | | FRFGHGDKVRYRCSSNLVLTGSSEREC |
| | | | | QGNGVWSGTEPICRQPYSYDFPEDVAP |
| | | | | ALGTSFSHMLGATNPTQKTKESLGRKI |
| | | | | QIQRSGHLNLYLLLDCSQSVSENDFLIF |
| | | | | KESASLMVDRIFSFEINVSVAIITFASEPK |
| | | | | VLMSVLNDNSRDMTEVISSLENANYKD |
| | | | | HENGTGTNTYAALNSVYLMMNNQMR |
| | | | | LLGMETMAWQEIRHAIILLTDGKSNMG |
| | | | | GSPKTAVDHIREILNINQKRNDYLDIYAI |
| | | | | GVGKLDVDWRELNELGSKKDGERHAF |
| | | | | ILQDTKALHQVFEHMLDVSKLTDTICG |
| | | | | VGNMSANASDQERTPWHVTIKPKSQET |
| | | | | CRGALISDQWVLTAAHCFRDGNDHSL |
| | | | | WRVNVGDPKSQWGKEFLIEKAVISPGF |
| | | | | DVFAKKNQGILEFYGDDIALLKLAQKV |
| | | | | KMSTHARPICLPCTMEANLALRRPQGS |
| | | | | TCRDHENELLNKQSVPAHFVALNGSKL |
| | | | | NINLKMGVEWTSCAEVVSQEKTMFPNL |
| | | | | TDVREVVTDQFLCSGTQEDESPCKGES |
| | | | | GGAVFLERRFRFFQVGLVSWGLYNPCL |
| | | | | GSADKNSRKRAPRSKVPPPRDFHINLFR |
| | | | | MQPWLRQHLGDVLNFLPL |
| 21 | AGP1 | Alpha-1-acid | P02763 | MALSWVLTVLSLLPLLEAQIPLCANLVP |
| | | glycoprotein 1 | | VPITNATLDQITGKWFYIASAFRNEEYN |
| | | | | KSVQEIQATFFYFTPNKTEDTIFLREYQT |
| | | | | RQDQCIYNTTYLNVQRENGTISRYVGG |
| | | | | QEHFAHLLILRDTKTYMLAFDVNDEKN |
| | | | | WGLSVYADKPETTKEQLGEFYEALDCL |
| | | | | RIPKSDVVYTDWKKDKCEPLEKQHEKE |
| | | | | RKQEEGES |
| 22 | AACT | Alpha-1- | P01011 | MERMLPLLALGLLAAGFCPAVLCHPNS |
| | | antichymotrypsin | | PLDEENLTQENQDRGTHVDLGLASANV |
| | | | | DFAFSLYKQLVLKAPDKNVIFSPLSISTA |
| | | | | LAFLSLGAHNTTLTEILKGLKFNLTETS |
| | | | | EAEIHQSFQHLLRTLNQSSDELQLSMGN |
| | | | | AMFVKEQLSLLDRFTEDAKRLYGSEAF |
| | | | | ATDFQDSAAAKKLINDYVKNGTRGKIT |
| | | | | DLIKDLDSQTMMVLVNYIFFKAKWEM |
| | | | | PFDPQDTHQSRFYLSKKKWVMVPMMS |
| | | | | LHHLTIPYFRDEELSCTVVELKYTGNAS |
| | | | | ALFILPDQDKMEEVEAMLLPETLKRWR |
| | | | | DSLEFREIGELYLPKFSISRDYNLNDILL |
| | | | | QLGIEEAFTSKADLSGITGARNLAVSQV |
| | | | | VHKAVLDVFEEGTEASAATAVKITLLS |
| | | | | ALVETRTIVRFNRPFLMIIVPTDTQNIFF |
| | | | | MSKVTNPKQA |
| 23 | A2MG | Alpha-2- | P01023 | MGKNKLLHPSLVLLLLVLLPTDASVSG |
| | | macroglobulin | | KPQYMVLVPSLLHTETTEKGCVLLSYL |
| | | | | NETVTVSASLESVRGNRSLFTDLEAEND |
| | | | | VLHCVAFAVPKSSSNEEVMFLTVQVKG |
| | | | | PTQEFKKRTTVMVKNEDSLVFVQTDKS |
| | | | | IYKPGQTVKFRVVSMDENFHPLNELIPL |
| | | | | VYIQDPKGNRIAQWQSFQLEGGLKQFS |
| | | | | FPLSSEPFQGSYKVVVQKKSGGRTEHPF |
| | | | | TVEEFVLPKFEVQVTVPKIITILEEEMNV |
| | | | | SVCGLYTYGKPVPGHVTVSICRKYSDA |
| | | | | SDCHGEDSQAFCEKFSGQLNSHGCFYQ |
| | | | | QVKTKVFQLKRKEYEMKLHTEAQIQEE |
| | | | | GTVVELTGRQSSEITRTITKLSFVKVDS |
| | | | | HFRQGIPFFGQVRLVDGKGVPIPNKVIFI |
| | | | | RGNEANYYSNATTDEHGLVQFSINTTN |
| | | | | VMGTSLTVRVNYKDRSPCYGYQWVSE |
| | | | | EHEEAHHTAYLVFSPSKSFVHLEPMSHE |
| | | | | LPCGHTQTVQAHYILNGGTLLGLKKLS |
| | | | | FYYLIMAKGGIVRTGTHGLLVKQEDMK |
| | | | | GHFSISIPVKSDIAPVARLLIYAVLPTGD |
| | | | | VIGDSAKYDVENCLANKVDLSFSPSQSL |
| | | | | PASHAHLRVTAAPQSVCALRAVDQSVL |
| | | | | LMKPDAELSASSVYNLLPEKDLTGFPGP |
| | | | | LNDQDNEDCINRHNVYINGITYTPVSST |
| | | | | NEKDMYSFLEDMGLKAFTNSKIRKPKM |
| | | | | CPQLQQYEMHGPEGLRVGFYESDVMG |
| | | | | RGHARLVHVEEPHTETVRKYFPETWIW |
| | | | | DLVVVNSAGVAEVGVTVPDTITEWKA |
| | | | | GAFCLSEDAGLGISSTASLRAFQPFFVEL |
| | | | | TMPYSVIRGEAFTLKATVLNYLPKCIRV |
| | | | | SVQLEASPAFLAVPVEKEQAPHCICANG |
| | | | | RQTVSWAVTPKSLGNVNFTVSAEALES |
| | | | | QELCGTEVPSVPEHGRKDTVIKPLLVEP |
| | | | | EGLEKETTFNSLLCPSGGEVSEELSLKLP |
| | | | | PNVVEESARASVSVLGDILGSAMQNTQ |
| | | | | NLLQMPYGCGEQNMVLFAPNIYVLDY |
| | | | | LNETQQLTPEIKSKAIGYLNTGYQRQLN |
| | | | | YKHYDGSYSTFGERYGRNQGNTWLTA |
| | | | | FVLKTFAQARAYIFIDEAHITQALIWLS |
| | | | | QRQKDNGCFRSSGSLLNNAIKGGVEDE |
| | | | | VTLSAYITIALLEIPLTVTHPVVRNALFC |
| | | | | LESAWKTAQEGDHGSHVYTKALLAYA |
| | | | | FALAGNQDKRKEVLKSLNEEAVKKDN |
| | | | | SVHWERPQKPKAPVGHFYEPQAPSAEV |
| | | | | EMTSYVLLAYLTAQPAPTSEDLTSATNI |
| | | | | VKWITKQQNAQGGFSSTQDTVVALHA |
| | | | | LSKYGAATFTRTGKAAQVTIQSSGTFSS |
| | | | | KFQVDNNNRLLLQQVSLPELPGEYSMK |
| | | | | VTGEGCVYLQTSLKYNILPEKEEFPFAL |
| | | | | GVQTLPQTCDEPKAHTSFQISLSVSYTG |
| | | | | SRSASNMAIVDVKMVSGFIPLKPTVKM |
| | | | | LERSNHVSRTEVSSNHVLIYLDKVSNQT |
| | | | | LSLFFTVLQDVPVRDLKPAIVKVYDYY |
| | | | | ETDEFAIAEYNAPCSKDLGNA |
| 24 | A1AT | Alpha-1- | P01009 | MPSSVSWGILLLAGLCCLVPVSLAEDPQ |
| | | antitrypsin | | GDAAQKTDTSHHDQDHPTFNKITPNLA |
| | | | | EFAFSLYRQLAHQSNSTNIFFSPVSIATA |
| | | | | FAMLSLGTKADTHDEILEGLNFNLTEIP |
| | | | | EAQIHEGFQELLRTLNQPDSQLQLTTGN |
| | | | | GLFLSEGLKLVDKFLEDVKKLYHSEAF |
| | | | | TVNFGDTEEAKKQINDYVEKGTQGKIV |
| | | | | DLVKELDRDTVFALVNYIFFKGKWERP |
| | | | | FEVKDTEEEDFHVDQVTTVKVPMMKR |
| | | | | LGMFNIQHCKKLSSWVLLMKYLGNAT |
| | | | | AIFFLPDEGKLQHLENELTHDIITKFLEN |
| | | | | EDRRSASLHLPKLSITGTYDLKSVLGQL |
| | | | | GITKVFSNGADLSGVTEEAPLKLSKAV |
| | | | | HKAVLTIDEKGTEAAGAMFLEAIPMSIP |
| | | | | PEVKFNKPFVFLMIEQNTKSPLFMGKV |
| | | | | VNPTQK |
| 25 | VTNC | Vitronectin | P04004 | MAPLRPLLILALLAWVALADQESCKGR |
| | | | | CTEGFNVDKKCQCDELCSYYQSCCTDY |
| | | | | TAECKPQVTRGDVFTMPEDEYTVYDD |
| | | | | GEEKNNATVHEQVGGPSLTSDLQAQSK |
| | | | | GNPEQTPVLKPEEEAPAPEVGASKPEGI |
| | | | | DSRPETLHPGRPQPPAEEELCSGKPFDA |
| | | | | FTDLKNGSLFAFRGQYCYELDEKAVRP |
| | | | | GYPKLIRDVWGIEGPIDAAFTRINCQGK |
| | | | | TYLFKGSQYWRFEDGVLDPDYPRNISD |
| | | | | GFDGIPDNVDAALALPAHSYSGRERVY |
| | | | | FFKGKQYWEYQFQHQPSQEECEGSSLS |
| | | | | AVFEHFAMMQRDSWEDIFELLFWGRTS |
| | | | | AGTRQPQFISRDWHGVPGQVDAAMAG |
| | | | | RIYISGMAPRPSLAKKQRFRHRNRKGY |
| | | | | RSQRGHSRGRNQNSRRPSRATWLSLFS |
| | | | | SEESNLGANNYDDYRMDWLVPATCEPI |
| | | | | QSVFFFSGDKYYRVNLRTRRVDTVDPP |
| | | | | YPRSIAQYWLGCPAPGHL |
| 26 | IGG2 | Immunoglobulin | P01859 | ASTKGPSVFPLAPCSRSTSESTAALGCL |
| | | heavy | | VKDYFPEPVTVSWNSGALTSGVHTFPA |
| | | constant | | VLQSSGLYSLSSVVTVPSSNFGTQTYTC |
| | | gamma 2 | | NVDHKPSNTKVDKTVERKCCVECPPCP |
| | | | | APPVAGPSVFLFPPKPKDTLMISRTPEVT |
| | | | | CVVVDVSHEDPEVQFNWYVDGVEVHN |
| | | | | AKTKPREEQFNSTFRVVSVLTVVHQDW |
| | | | | LNGKEYKCKVSNKGLPAPIEKTISKTKG |
| | | | | QPREPQVYTLPPSREEMTKNQVSLTCL |
| | | | | VKGFYPSDISVEWESNGQPENNYKTTPP |
| | | | | MLDSDGSFFLYSKLTVDKSRWQQGNV |
| | | | | FSCSVMHEALHNHYTQKSLSLSPGK |
| 27 | CFAH | Complement | P08603 | MRLLAKIICLMLWAICVAEDCNELPPRR |
| | | Factor H | | NTEILTGSWSDQTYPEGTQAIYKCRPGY |
| | | | | RSLGNVIMVCRKGEWVALNPLRKCQK |
| | | | | RPCGHPGDTPFGTFTLTGGNVFEYGVK |
| | | | | AVYTCNEGYQLLGEINYRECDTDGWT |
| | | | | NDIPICEVVKCLPVTAPENGKIVSSAME |
| | | | | PDREYHFGQAVRFVCNSGYKIEGDEEM |
| | | | | HCSDDGFWSKEKPKCVEISCKSPDVING |
| | | | | SPISQKIIYKENERFQYKCNMGYEYSER |
| | | | | GDAVCTESGWRPLPSCEEKSCDNPYIPN |
| | | | | GDYSPLRIKHRTGDEITYQCRNGFYPAT |
| | | | | RGNTAKCTSTGWIPAPRCTLKPCDYPDI |
| | | | | KHGGLYHENMRRPYFPVAVGKYYSYY |
| | | | | CDEHFETPSGSYWDHIHCTQDGWSPAV |
| | | | | PCLRKCYFPYLENGYNQNYGRKFVQG |
| | | | | KSIDVACHPGYALPKAQTTVTCMENG |
| | | | | WSPTPRCIRVKTCSKSSIDIENGFISESQY |
| | | | | TYALKEKAKYQCKLGYVTADGETSGSI |
| | | | | TCGKDGWSAQPTCIKSCDIPVFMNART |
| | | | | KNDFTWFKLNDTLDYECHDGYESNTG |
| | | | | STTGSIVCGYNGWSDLPICYERECELPKI |
| | | | | DVHLVPDRKKDQYKVGEVLKFSCKPG |
| | | | | FTIVGPNSVQCYHFGLSPDLPICKEQVQ |
| | | | | SCGPPPELLNGNVKEKTKEEYGHSEVV |
| | | | | EYYCNPRFLMKGPNKIQCVDGEWTTLP |
| | | | | VCIVEESTCGDIPELEHGWAQLSSPPYY |
| | | | | YGDSVEFNCSESFTMIGHRSITCIHGVW |
| | | | | TQLPQCVAIDKLKKCKSSNLIILEEHLK |
| | | | | NKKEFDHNSNIRYRCRGKEGWIHTVCI |
| | | | | NGRWDPEVNCSMAQIQLCPPPPQIPNSH |
| | | | | NMTTTLNYRDGEKVSVLCQENYLIQEG |
| | | | | EEITCKDGRWQSIPLCVEKIPCSQPPQIE |
| | | | | HGTINSSRSSQESYAHGTKLSYTCEGGF |
| | | | | RISEENETTCYMGKWSSPPQCEGLPCKS |
| | | | | PPEISHGVVAHMSDSYQYGEEVTYKCF |
| | | | | EGFGIDGPAIAKCLGEKWSHPPSCIKTD |
| | | | | CLSLPSFENAIPMGEKKDVYKAGEQVT |
| | | | | YTCATYYKMDGASNVTCINSRWTGRP |
| | | | | TCRDTSCVNPPTVQNAYIVSRQMSKYP |
| | | | | SGERVRYQCRSPYEMFGDEEVMCLNG |
| | | | | NWTEPPQCKDSTGKCGPPPPIDNGDITS |
| | | | | FPLSVYAPASSVEYQCQNLYQLEGNKRI |
| | | | | TCRNGQWSEPPKCLHPCVISREIMENYN |
| | | | | IALRWTAKQKLYSRTGESVEFVCKRGY |
| | | | | RLSSRSHTLRTTCWDGKLEYPTCAKR |
| 28 | APOH | Beta-2- | P02749 | MISPVLILFSSFLCHVAIAGRTCPKPDDL |
| | | glycoprotein1 | | PFSTVVPLKTFYEPGEEITYSCKPGYVSR |
| | | | | GGMRKFICPLTGLWPINTLKCTPRVCPF |
| | | | | AGILENGAVRYTTFEYPNTISFSCNTGF |
| | | | | YLNGADSAKCTEEGKWSPELPVCAPIIC |
| | | | | PPPSIPTFATLRVYKPSAGNNSLYRDTA |
| | | | | VFECLPQHAMFGNDTITCTTHGNWTKL |
| | | | | PECREVKCPFPSRPDNGFVNYPAKPTLY |
| | | | | YKDKATFGCHDGYSLDGPEEIECTKLG |
| | | | | NWSAMPSCKASCKVPVKKATVVYQGE |
| | | | | RVKIQEKFKNGMLHGDKVSFFCKNKEK |
| | | | | KCSYTEDAQCIDGTIEVPKCFKEHSSLA |
| | | | | FWKTDASDVKPC |
| 29 | APOD | Apolipoprotein | P05090 | MVMLLLLLSALAGLFGAAEGQAFHLG |
| | | D | | KCPNPPVQENFDVNKYLGRWYEIEKIPT |
| | | | | TFENGRCIQANYSLMENGKIKVLNQEL |
| | | | | RADGTVNQIEGEATPVNLTEPAKLEVK |
| | | | | FSWFMPSAPYWILATDYENYALVYSCT |
| | | | | CIIQLFHVDFAWILARNPNLPPETVDSL |
| | | | | KNILTSNNIDVKKMTVTDQVNCPKLS |
| 30 | TRFE | Serotransferrin | P02787 | MRLAVGALLVCAVLGLCLAVPDKTVR |
| | | | | WCAVSEHEATKCQSFRDHMKSVIPSDG |
| | | | | PSVACVKKASYLDCIRAIAANEADAVT |
| | | | | LDAGLVYDAYLAPNNLKPVVAEFYGS |
| | | | | KEDPQTFYYAVAVVKKDSGFQMNQLR |
| | | | | GKKSCHTGLGRSAGWNIPIGLLYCDLPE |
| | | | | PRKPLEKAVANFFSGSCAPCADGTDFP |
| | | | | QLCQLCPGCGCSTLNQYFGYSGAFKCL |
| | | | | KDGAGDVAFVKHSTIFENLANKADRD |
| | | | | QYELLCLDNTRKPVDEYKDCHLAQVPS |
| | | | | HTVVARSMGGKEDLIWELLNQAQEHF |
| | | | | GKDKSKEFQLFSSPHGKDLLFKDSAHG |
| | | | | FLKVPPRMDAKMYLGYEYVTAIRNLRE |
| | | | | GTCPEAPTDECKPVKWCALSHHERLKC |
| | | | | DEWSVNSVGKIECVSAETTEDCIAKIMN |
| | | | | GEADAMSLDGGFVYIAGKCGLVPVLAE |
| | | | | NYNKSDNCEDTPEAGYFAVAVVKKSA |
| | | | | SDLTWDNLKGKKSCHTAVGRTAGWNI |
| | | | | PMGLLYNKINHCRFDEFFSEGCAPGSK |
| | | | | KDSSLCKLCMGSGLNLCEPNNKEGYYG |
| | | | | YTGAFRCLVEKGDVAFVKHQTVPQNT |
| | | | | GGKNPDPWAKNLNEKDYELLCLDGTR |
| | | | | KPVEEYANCHLARAPNHAVVTRKDKE |
| | | | | ACVHKILRQQQHLFGSNVTDCSGNFCL |
| | | | | FRSETKDLLFRDDTVCLAKLHDRNTYE |
| | | | | KYLGEEYVKAVGNLRKCSTSSLLEACT |
| | | | | FRRP |
| 47 | FETUA | Alpha-2-HS- | P02765 | MKSLVLLLCLAQLWGCHSAPHGPGLIY |
| | | glycoprotein | | RQPNCDDPETEEAALVAIDYINQNLPW |
| | | | | GYKHTLNQIDEVKVWPQQPSGELFEIEI |
| | | | | DTLETTCHVLDPTPVARCSVRQLKEHA |
| | | | | VEGDCDFQLLKLDGKFSVVYAKCDSSP |
| | | | | DSAEDVRKVCQDCPLLAPLNDTRVVH |
| | | | | AAKAALAAFNAQNNGSNFQLEEISRAQ |
| | | | | LVPLPPSTYVEFTVSGTDCVAKEATEAA |
| | | | | KCNLLAEKQYGFCKATLSEKLGGAEVA |
| | | | | VTCMVFQTQPVSSQPQPEGANEAVPTP |
| | | | | VVDPDAPPSPPLGAPGLPPAGSPPDSHV |
| | | | | LLAAPPGHQLHRAHYDLRHTFMGVVS |
| | | | | LGSPSGEVSHPRKTRTVVQPSVGAAAG |
| | | | | PVVPPCPGRIRHFKV |
| 48 | A2GL | Leucine-rich | P02750 | MSSWSRQRPKSPGGIQPHVSRTLFLLLL |
| | | Alpha-2- | | LAASAWGVTLSPKDCQVFRSDHGSSIS |
| | | glycoprotein | | CQPPAEIPGYLPADTVHLAVEFFNLTHL |
| | | | | PANLLQGASKLQELHLSSNGLESLSPEF |
| | | | | LRPVPQLRVLDLTRNALTGLPPGLFQAS |
| | | | | ATLDTLVLKENQLEVLEVSWLHGLKAL |
| | | | | GHLDLSGNRLRKLPPGLLANFTLLRTLD |
| | | | | LGENQLETLPPDLLRGPLQLERLHLEGN |
| | | | | KLQVLGKDLLLPQPDLRYLFLNGNKLA |
| | | | | RVAAGAFQGLRQLDMLDLSNNSLASVP |
| | | | | EGLWASLGQPNWDMRDGFDISGNPWI |
| | | | | CDQNLSDLYRWLQAQKDKMFSQNDTR |
| | | | | CAGPEAVKGQTLLAVAKSQ |
| 49 | TTR | Transthyretin | P02766 | MASHRLLLLCLAGLVFVSEAGPTGTGE |
| | | | | SKCPLMVKVLDAVRGSPAINVAVHVFR |
| | | | | KAADDTWEPFASGKTSESGELHGLTTE |
| | | | | EEFVEGIYKVEIDTKSYWKALGISPFHE |
| | | | | HAEVVFTANDSGPRRYTIAALLSPYSYS |
| | | | | TTAVVTNPKE |
| 50 | AFAM | Afamin | P43652 | MKLLKLTGFIFFLFFLTESLTLPTQPRDI |
| | | | | ENFNSTQKFIEDNIEYITIIAFAQYVQEA |
| | | | | TFEEMEKLVKDMVEYKDRCMADKTLP |
| | | | | ECSKLPNNVLQEKICAMEGLPQKHNFS |
| | | | | HCCSKVDAQRRLCFFYNKKSDVGFLPP |
| | | | | FPTLDPEEKCQAYESNRESLLNHFLYEV |
| | | | | ARRNPFVFAPTLLTVAVHFEEVAKSCC |
| | | | | EEQNKVNCLQTRAIPVTQYLKAFSSYQ |
| | | | | KHVCGALLKFGTKVVHFIYIAILSQKFP |
| | | | | KIEFKELISLVEDVSSNYDGCCEGDVVQ |
| | | | | CIRDTSKVMNHICSKQDSISSKIKECCEK |
| | | | | KIPERGQCIINSNKDDRPKDLSLREGKFT |
| | | | | DSENVCQERDADPDTFFAKFTFEYSRR |
| | | | | HPDLSIPELLRIVQIYKDLLRNCCNTENP |
| | | | | PGCYRYAEDKFNETTEKSLKMVQQEC |
| | | | | KHFQNLGKDGLKYHYLIRLTKIAPQLST |
| | | | | EELVSLGEKMVTAFTTCCTLSEEFACV |
| | | | | DNLADLVFGELCGVNENRTINPAVDHC |
| | | | | CKTNFAFRRPCFESLKADKTYVPPPFSQ |
| | | | | DLFTFHADMCQSQNEELQRKTDRFLVN |
| | | | | LVKLKHELTDEELQSLFTNFANVVDKC |
| | | | | CKAESPEVCFNEESPKIGN |
| 51 | APOA1 | Apolipoprotein | P02647 | MKAAVLTLAVLFLTGSQARHFWQQDE |
| | | A-I | | PPQSPWDRVKDLATVYVDVLKDSGRD |
| | | | | YVSQFEGSALGKQLNLKLLDNWDSVTS |
| | | | | TFSKLREQLGPVTQEFWDNLEKETEGL |
| | | | | RQEMSKDLEEVKAKVQPYLDDFQKKW |
| | | | | QEEMELYRQKVEPLRAELQEGARQKL |
| | | | | HELQEKLSPLGEEMRDRARAHVDALRT |
| | | | | HLAPYSDELRQRLAARLEALKENGGAR |
| | | | | LAEYHAKATEHLSTLSEKAKPALEDLR |
| | | | | QGLLPVLESFKVSFLSALEEYTKKLNTQ |
| 52 | C4BPA | C4 b-binding | P04003 | MHPPKTPSGALHRKRKMAAWPFSRLW |
| | | protein alpha | | KVSDPILFQMTLIAALLPAVLGNCGPPP |
| | | chain | | TLSFAAPMDITLTETRFKTGTTLKYTCL |
| | | | | PGYVRSHSTQTLTCNSDGEWVYNTFCI |
| | | | | YKRCRHPGELRNGQVEIKTDLSFGSQIE |
| | | | | FSCSEGFFLIGSTTSRCEVQDRGVGWSH |
| | | | | PLPQCEIVKCKPPPDIRNGRHSGEENFY |
| | | | | AYGFSVTYSCDPRFSLLGHASISCTVEN |
| | | | | ETIGVWRPSPPTCEKITCRKPDVSHGEM |
| | | | | VSGFGPIYNYKDTIVFKCQKGFVLRGSS |
| | | | | VIHCDADSKWNPSPPACEPNSCINLPDIP |
| | | | | HASWETYPRPTKEDVYVVGTVLRYRC |
| | | | | HPGYKPTTDEPTTVICQKNLRWTPYQG |
| | | | | CEALCCPEPKLNNGEITQHRKSRPANHC |
| | | | | VYFYGDEISFSCHETSRFSAICQGDGTW |
| | | | | SPRTPSCGDICNFPPKIAHGHYKQSSSYS |
| | | | | FFKEEIIYECDKGYILVGQAKLSCSYSH |
| | | | | WSAPAPQCKALCRKPELVNGRLSVDK |
| | | | | DQYVEPENVTIQCDSGYGVVGPQSITCS |
| | | | | GNRTWYPEVPKCEWETPEGCEQVLTG |
| | | | | KRLMQCLPNPEDVKMALEVYKLSLEIE |
| | | | | QLELQRDSARQSTLDKEL |
| 66 | AGP2 | Alpha-1-acid | P19652 | MALSWVLTVLSLLPLLEAQIPLCANLVP |
| | | glycoprotein 2 | | VPITNATLDRITGKWFYIASAFRNEEYN |
| | | | | KSVQEIQATFFYFTPNKTEDTIFLREYQT |
| | | | | RQNQCFYNSSYLNVQRENGTVSRYEGG |
| | | | | REHVAHLLFLRDTKTLMFGSYLDDEKN |
| | | | | WGLSFYADKPETTKEQLGEFYEALDCL |
| | | | | CIPRSDVMYTDWKKDKCEPLEKQHEKE |
| | | | | RKQEEGES |
| 67 | KNG1 | Kininogen-1 | P01042 | MKLITILFLCSRLLLSLTQESQSEEIDCN |
| | | | | DKDLFKAVDAALKKYNSQNQSNNQFV |
| | | | | LYRITEATKTVGSDTFYSFKYEIKEGDC |
| | | | | PVQSGKTWQDCEYKDAAKAATGECTA |
| | | | | TVGKRSSTKFSVATQTCQITPAEGPVVT |
| | | | | AQYDCLGCVHPISTQSPDLEPILRHGIQ |
| | | | | YFNNNTQHSSLFMLNEVKRAQRQVVA |
| | | | | GLNFRITYSIVQTNCSKENFLFLTPDCKS |
| | | | | LWNGDTGECTDNAYIDIQLRIASFSQNC |
| | | | | DIYPGKDFVQPPTKICVGCPRDIPTNSPE |
| | | | | LEETLTHTITKLNAENNATFYFKIDNVK |
| | | | | KARVQVVAGKKYFIDFVARETTCSKES |
| | | | | NEELTESCETKKLGQSLDCNAEVYVVP |
| | | | | WEKKIYPTVNCQPLGMISLMKRPPGFSP |
| | | | | FRSSRIGEIKEETTVSPPHTSMAPAQDEE |
| | | | | RDSGKEQGHTRRHDWGHEKQRKHNLG |
| | | | | HGHKHERDQGHGHQRGHGLGHGHEQ |
| | | | | QHGLGHGHKFKLDDDLEHQGGHVLDH |
| | | | | GHKHKHGHGHGKHKNKGKKNGKHNG |
| | | | | WKTEHLASSSEDSTTPSAQTQEKTEGPT |
| | | | | PIPSLAKPGVTVTFSDFQDSDLIATMMP |
| | | | | PISPAPIQSDDDWIPDIQIDPNGLSFNPIS |
| | | | | DFPDTTSPKCPGRPWKSVSEINPTTQMK |
| | | | | ESYYFDLTDGLS |

Table 7 identifies and defines the glycan symbol structures included in Tables 1, 2, 3, 3B, 3C, and 3D. Table 7 identifies a coded representation of the composition for each glycan structure included in Tables 1, 2, 3, 3B, 3C, and 3D. As used herein, the 4-digit GL NO. is a designation that represents the number of hexoses, the number of HexNAcs, the number of Fucoses, and the number of Neuraminic Acids. It should be noted that glycan structure GL No 1102 is an O-glycan and the remaining glycans of Table 7 were N-glycans.

TABLE 7
Glycan Structure GL NOS: Composition

| Glycan | | | Glycan |
|---|---|---|---|
| Structure | | Com- | Molecular |
| GL NO. | Symbol Structure | position | Weight |
| 1102 | 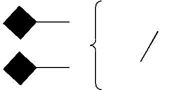 | Hex(1) Hex- NAc(1) Fuc(0) Neu- Ac(2) | 947.323008 |
| 3400 | 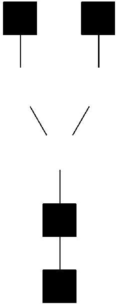 | Hex(3) Hex- NAc(4) Fuc(0) Neu- Ac(0) | 1298.47593 |
| 3410 | 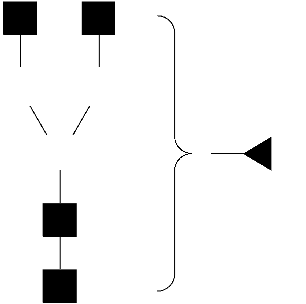 | Hex(3) Hex- NAc(4) Fuc(1) Neu- Ac(0) | 1444.53384 |
| 3510 | 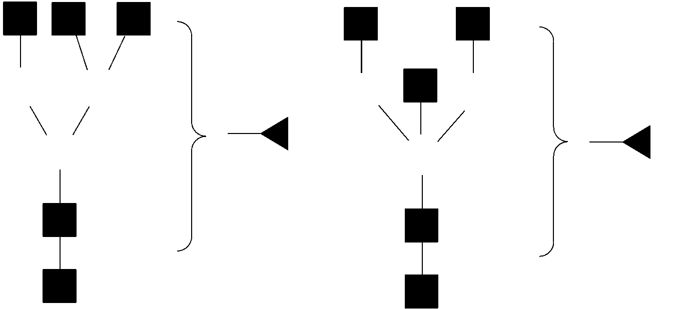 | Hex(3) Hex- NAc(5) Fuc(1) Neu- Ac(0) | 1647.61321 |
| 4300 | 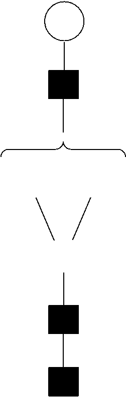 | Hex(4) Hex- NAc(3) Fuc(0) Neu- Ac(0) | 1257.44938 |
| 4510 | 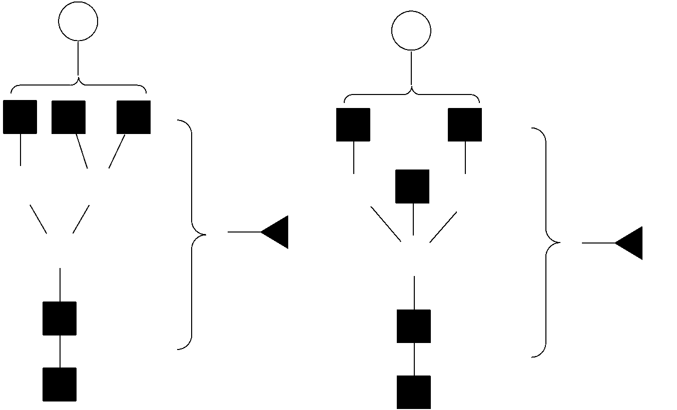 | Hex(4) Hex- NAc(5) Fuc(1) Neu- Ac(0) | 1809.66603 |
| 4511 | 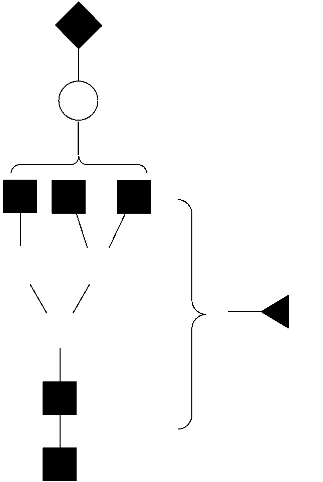 | Hex(4) Hex- NAc(5) Fuc(1) Neu- Ac(1) | 2100.76144 |
| 5200 | 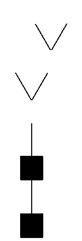 | Hex(5) Hex- NAc(2) Fuc(0) Neu- Ac(0) | 1216.42284 |
| 5301 | 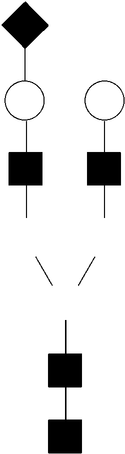 | Hex(5) Hex- NAc(3) Fuc(0) Neu- Ac(1) | 1710.59761 |
| 5400 | 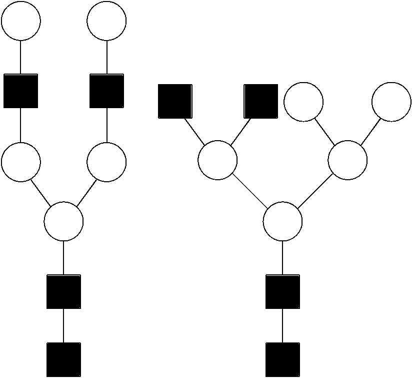 | Hex(5) Hex- NAc(4) Fuc(0) Neu- Ac(0) | 1622.58157 |
| 5401 | 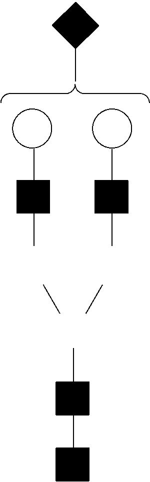 | Hex(5) Hex- NAc(4) Fuc(0) Neu- Ac(1) | 1913.67698 |
| 5402 | 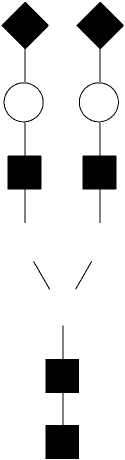 | Hex(5) Hex- NAc(4) Fuc(0) Neu- Ac(2) | 2204.77239 |
| 5411 | 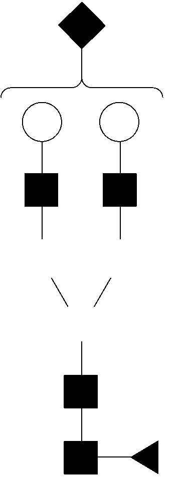 | Hex(5) Hex- NAc(4) Fuc(1) Neu- Ac(1) | 2059.73489 |
| 5412 | 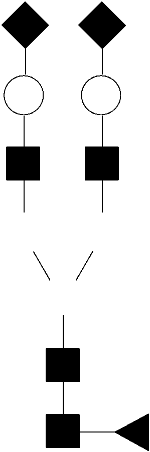 | Hex(5) Hex- NAc(4) Fuc(1) Neu- Ac(2) | 2350.8303 |
| 5421 | 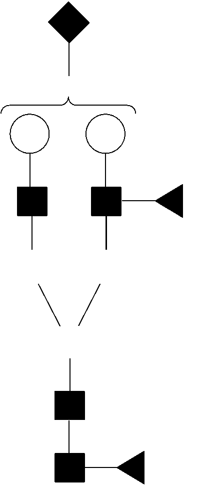 | Hex(5) Hex- NAc(4) Fuc(2) Neu- Ac(1) | 2205.79279 |
| 5510 | 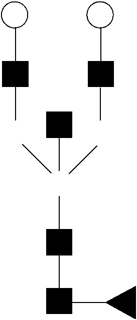 | Hex(5) Hex- NAc(5) Fuc(1) Neu- Ac(0) | 1971.71885 |
| 6501 | 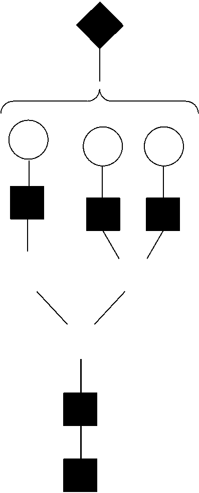 | Hex(6) Hex- NAc(5) Fuc(0) Neu- Ac(1) | 2278.80917 |
| 6502 | 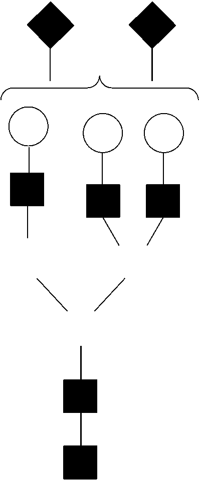 | Hex(6) Hex- NAc(5) Fuc(0) Neu- Ac(2) | 2569.90458 |
| 6503 | 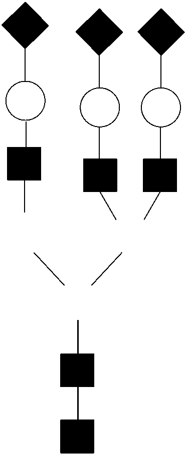 | Hex(6) Hex- NAc(5) Fuc(0) Neu- Ac(3) | 2860.99999 |
| 6511 | 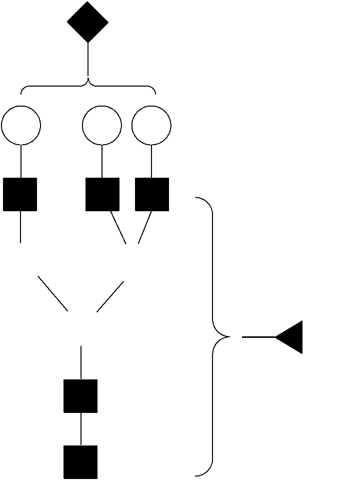 | Hex(6) Hex- NAc(5) Fuc(1) Neu- Ac(1) | 2424.86708 |
| 6512 | 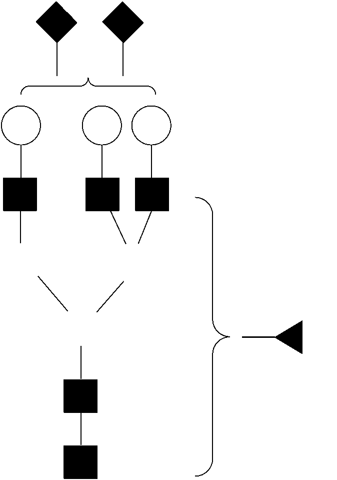 | Hex(6) Hex- NAc(5) Fuc(1) Neu- Ac(2) | 2715.962 |
| 6513 | 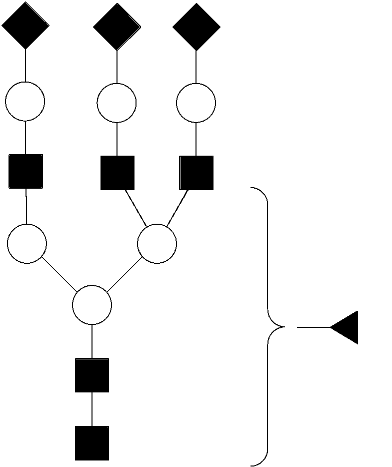 | Hex(6) Hex- NAc(5) Fuc(1) Neu- Ac(3) | 3007.057896 |
| 6521 | 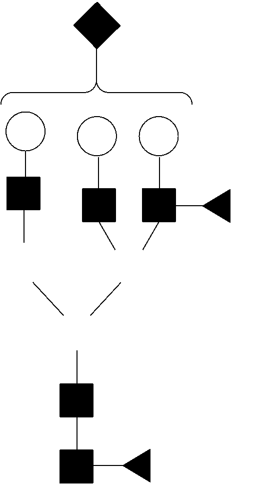 | Hex(6) Hex- NAc(5) Fuc(2) Neu- Ac(1) | |
| 7602 | 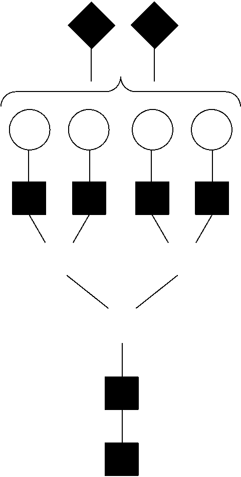 | Hex(7) Hex- NAc(6) Fuc(0) Neu- Ac(2) | 2935.03677 |
| 7603 | 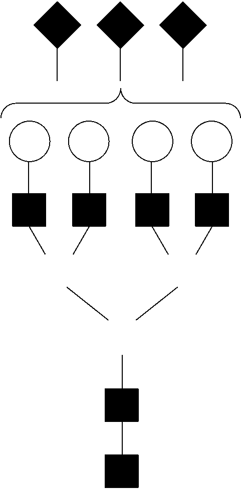 | Hex(7) Hex- NAc(6) Fuc(0) Neu- Ac(3) | 3226.13218 |
| 7604 | 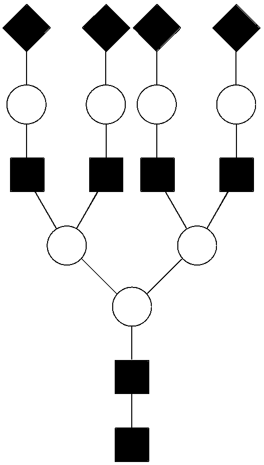 | Hex(7) Hex- NAc(6) Fuc(0) Neu- Ac(4) | 3517.227588 |
| 7612 | 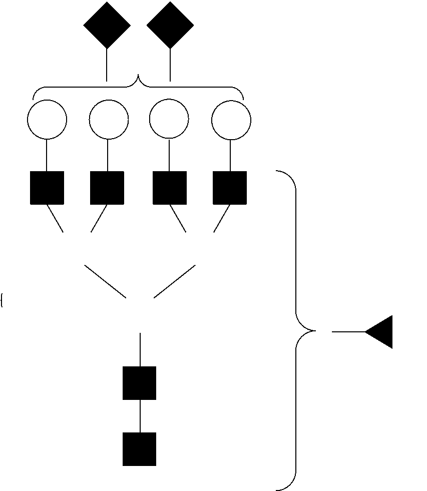 | Hex(7) Hex- NAc(6) Fuc(1) Neu(5) Ac(2) | 3081.09467 |
| 7613 | 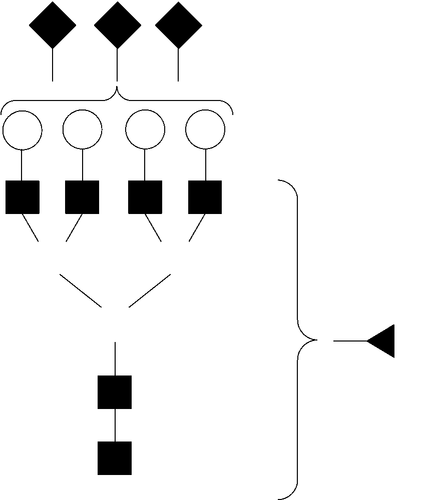 | Hex(7) Hex- NAc(6) Fuc(1) Neu(5) Ac(3) | 3372.190084 |
| 7614 | 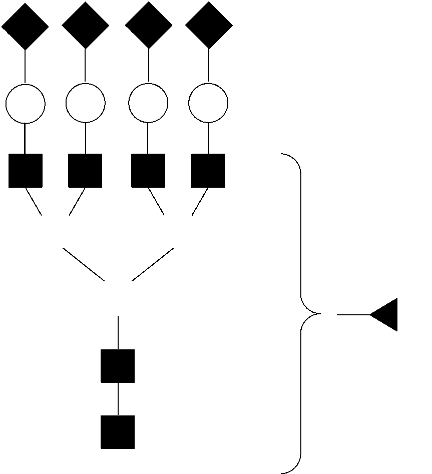 | Hex(7) Hex- NAc(6) Fuc(1) Neu- Ac(4) | 3663.285494 |
| 8704 | 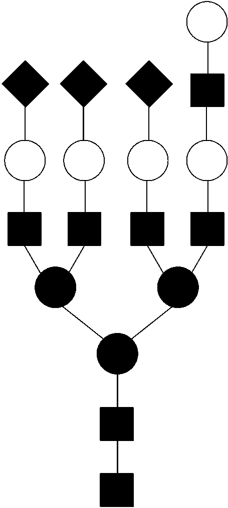 | Hex(8) Hex- NAc(7) Fuc(0) Neu- Ac(4) | 3882.35978 |
| 9804 | 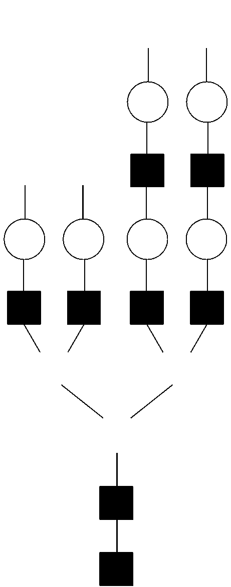 | Hex(9) Hex- NAc(8) Fuc(0) Neu- Ac(4) | 4247.49196 |
| 11915 | 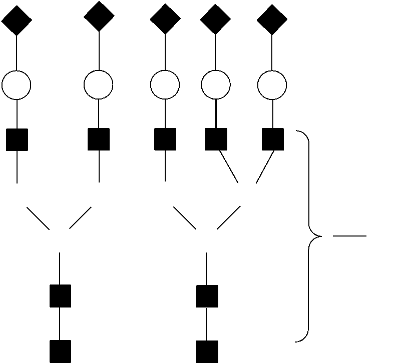 | Hex(11) Hex- NAc(9) Fuc(1) Neu- Ac(5) | 5211.830288 |
| 121005 | 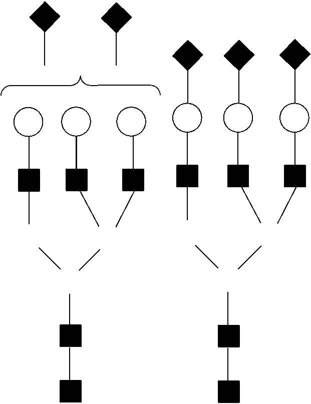 | Hex(12) Hex- NAc(10) Fuc(0) Neu- Ac(5) | 5430.90457 |

Legend for Table 7

[symbol]

Glc

[symbol]

Gal

[symbol]

Man

[symbol]

Fuc

[symbol]

Neu5Ac

[symbol]

GlcNAc

[symbol]

GalNAc

[symbol]

ManNAc

Table 7 illustrates the symbol structure and composition of detected glycan moieties that correspond to glycopeptides of Table 1, 2, 3, 3B, 3C, and 3D based on the Glycan GL NO. The term Symbol Structure illustrates a geometric linking structure of the carbohydrates where the bottommost carbohydrate such as N-acetylglucosamine is bound to the designated amino acid for an N-linked glycan and the rightmost carbohydrate such as N-acetylgalactosamine is bound to the designated amino acid for an O-linked glycan. It should be noted that the Glycan Structure GL NO 1102 is an O-linked glycan and that the rest of the glycans in Table 7 are N-linked glycans. For reference, N-linked glycans have a glycan attached to the amino acid asparagine and O-linked glycans have a glycan attached to either a serine or a threonine.

The identity of the various monosaccharides is illustrated by the Legend section located at the end of Table 7. The abbreviations of the Legend are Glc that represents glucose and is indicated by a dark circle, Gal that represents galactose and is indicated by an open circle, Man that represents mannose and is indicated by a circle with intermediate grey shading, Fuc that represents fucose and is indicated by a dark triangle, Neu5Ac that represents N-acetylneuraminic acid and is indicated by a dark diamond, GlcNAc that represents N-acetylglucosamine and is indicated by a dark square, GalNAc that represents N-acetylgalactosamine and is indicated by an open square, and ManNAc that represents N-acetylmannosamine and is indicated by a square with intermediate grey shading.

The term Composition refers to the number of various classes of carbohydrates that make up the glycan. The quantity for each class of carbohydrate is depicted as a number in parenthesis to the right of an abbreviation that corresponds to the class of the carbohydrate. The abbreviations for these classes are Hex, HexNAc, Fuc, and NeuAc that respectively correspond to hexose, N-acetylhexosamine, fucose, and N-acetylneuraminic acid. It should be noted that hexose sugars include glucose, galactose, and mannose; and N-acetylhexosamine sugars includes N-acetylglucosamine, N-acetylgalactosamine, and N-acetylmannosamine. In various embodiments, the terms Neu5Ac, NeuAc, and N-acetylneuraminic acid may be referred to as sialic acid.

Referring back to Table 7, for some entries, there are two symbol structures provided for one Glycan Structure GL NO such as, for example, Glycan Structure GL NO 3510. Thus, the identify of a peptide that references a Glycan Structure GL NO that has two symbol structures could be either one of the two possibilities based on the MRM of the LC-MS analysis. In some instances, a bracket symbol is used as part of the Symbol Structure to indicate that the precise bonding linkage is not exactly known, but that the linking line segment is attached to one of the plurality of adjacent carbohydrates immediately adjacent to the bracket. For example, the fucose of Glycan Structure GL NO 3510 could have either a core fucose or an outer-arm fucose linkage. In some instances, for fucosylated glycans illustrated in Table 7, the fucose orientation of either core or outer-arm linkage can be specified.

It should be noted that glycan symbol structure can illustrate an antennary format in the form of branches. For example, Glycan Structure GL NO's 6513 and 7604 show a tri-antennary and tetra-antennary sialic acid format, respectively.

Aspects of the disclosure include kits comprising one or more compositions, each comprising one or more peptide structures of the disclosure that can be used as assay standards, and instructions for use. Kits in accordance with one or more embodiments described herein may include a label indicating the intended use of the contents of the kit. The term “label” as used herein with respect to a kit includes any writing, or recorded material supplied on or with a kit, or that otherwise accompanies a kit.

The peptide structures and the transitions produced therefrom, as described herein, may be useful for diagnosing and treating an ovarian cancer disease state. A transition includes a precursor ion and at least one product ion grouping. As reviewed herein, the peptide structures in Tables 1, 2, 3, 3B, 3C, and 3D as well as their corresponding precursor ion and product ion groupings in Tables 4, 4B, and 4C (these ions having defined m/z ratios or m/z ratios that fall within the m/z ranges identified herein), can be used in mass spectrometry-based analyses to diagnose and facilitate treatment of diseases, such as, for example, PC.

Aspects of the disclosure include methods for analyzing one or more peptide structures, as described herein. In some embodiments, the methods involve processing a sample from a patient to generate a prepared sample that can be inputted into a mass spectrometry system (e.g., a reaction monitoring mass spectrometry system). In certain embodiments, processing the sample can comprise performing one or more of: a denaturation procedure, a reduction procedure, an alkylation procedure, and a digestion procedure. The denaturation and reduction procedures may be implemented in a manner similar to, for example, denaturation and reduction 202 in FIG. 2. The alkylation procedure may be implemented in a manner similar to, for example, alkylation procedure 204 in FIG. 2. The digestion procedure may be implemented in a manner similar to, for example, digestion procedure 206 in FIG. 2.

In some embodiments, the methods for analyzing one or more peptide structures involve detecting a set of product ions generated by a reaction monitoring mass spectrometry system in which one or more product ions may correspond to each of the one or more peptide structures that have been inputted into the mass spectrometry system. As described herein, each peptide structure can be converted into a set of product ions having a defined m/z ratio, as provided in Tables 4, 4B, and 4C or an m/z ratio within an identified m/z ratio as provided in Tables 4, 4B, and 4C. In some embodiments, the methods involve generating quantification (e.g., abundance) data for the one or more product ions detected using the reaction monitoring mass spectrometry system.

In some embodiments, the methods further comprise generating a diagnosis output using the quantification data and a model that has been trained using supervised or unsupervised machine learning. In certain embodiments, the reaction monitoring mass spectrometry system may include multiple/selected reaction monitoring mass spectrometry (MRM/SRM-MS) to detect the one or more product ions and generate the quantification data.

### VIII. Representative Experimental Results

### VIII.A. Exemplary Retrospective Analysis

### VIII.A. 1. Sample Acquisition

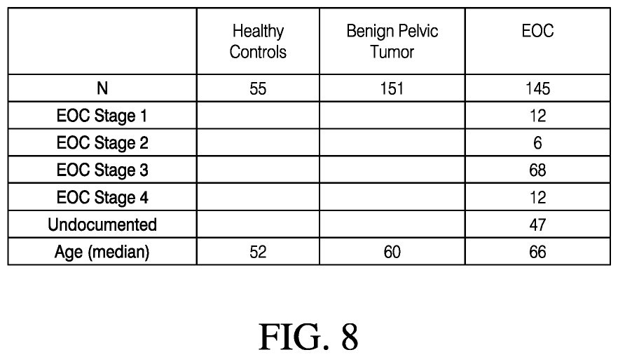

FIG. 8 is a table describing the distribution of the samples acquired in this exemplary retrospective analysis in accordance with one or more embodiments. As shown in FIG. 8, serum samples were acquired from a commercial biobank for 151 women with benign pelvic masses, 145 women with malignant epithelial ovarian cancer (EOC), and 55 healthy controls. Information on stage of EOC was available in 98 of the 145 patients with EOC. All samples were obtained prior to therapeutic intervention. Information on the benign or malignant nature of tumors was based on histopathological analysis of tissue specimens.

### VIII.A.2. Sample Processing

Sample processing involved pooled human serum/plasma (e.g., glycoprotein standards purified from human serum/plasma) for assay normalization, dithiothreitol (DTT), and iodoacetamide (IAA), sequencing-grade trypsin, LC-MS-grade water and acetonitrile, and formic acid (LC-MS grade). Serum samples were treated with DTT and IAA to reduce disulfide bonds and to inhibit cysteine proteases, respectively, followed by digestion with trypsin at 37 °C for 18 hours. The digestion was quenched by adding formic acid to each sample to a final concentration of 1% (v/v).

LC-MS analysis included separating digested serum samples over an Agilent ZORBAX Eclipse Plus C18 column (2.1 mm×150 mm i.d., 1.8 μm particle size) using an Agilent 1290 Infinity UHPLC system. The mobile phase A consisted of 3% acetonitrile, 0.1% formic acid in water (v/v), and the mobile phase B of 90% acetonitrile 0.1% formic acid in water (v/v), with the flow rate set at 0.5 mL/minute. The binary solvent composition was set at 100% mobile phase A at the beginning of the run, linearly shifting to 20% B at 20 minutes, 30% B at 40 minutes, and 44% B at 47 minutes. The column was flushed with 100% B and equilibrated with 100% A for a total run time of 70 minutes. After electrospray ionization, operated in positive ion mode, samples were injected into an Agilent 6495B triple quadrupole MS operated in dynamic multiple reaction monitoring (dMRM) mode. The MRM transitions comprised 513 glycopeptide structures which were normalized by comparing them with the abundance of 71 non-glycosylated peptide structures, representing each of 71 proteins from which the glycopeptides monitored were derived. Samples were injected randomized as to underlying phenotype, and reference pooled serum digests were injected interspersed with study samples.

### VIII.A.3. Data Analysis

Analysis resulted in 683 peptide structures (both peptide and glycopeptide isoforms) being reflected by 1106 MRM transitions, representing 71 high-abundance (concentrations ≃of 10 μg/ml) serum glycoproteins. Our transition list consisted of glycopeptides and non-glycosylated peptides from each glycoprotein. A spectrogram feature recognition and integration software based on recurrent neural networks was used to integrate chromatogram peaks and to obtain molecular abundance quantification for each peptide structure.

Normalized abundances of peptide structures, corrected for within-run drift, were assessed in samples from healthy controls, patients with benign pelvic tumors and those with EOC. Raw abundances were normalized by using spiked-in heavy-isotope-labeled internal standards with known peptide concentrations. The calculation relies either on relative abundance or on site occupancy, i.e., on the fractional abundance across all glycans observed at that site. Log-transformed concentration-normalized data for 501 glycopeptide structures (452 of which are based on on-site occupancy and 49 on relative abundance) and for 70 aglycosylated peptide structures were ultimately used for the analysis, totaling 571 unique peptide structures. Fold changes for individual peptide structures were calculated on normalized abundances of healthy (control) vs. EOC samples and benign tumor vs. EOC samples. False discovery rates (FDR) were calculated using the Benjamini-Hochberg method. Principal component analysis (PCA) was performed on log-concentration-normalized abundances of glycopeptide structures to investigate differences among the three phenotypes (e.g., healthy control, EOC, and benign pelvic tumor) studied. Prior to performing PCA, normalized abundances were scaled such that the distributions of all biomarkers were Gaussian with zero mean and unit variance.

To compare any two phenotypes, age-adjusted linear regression was used on a feature-by-feature basis with phenotype serving as the sole binary independent variable. Correcting for multiple comparisons, differences of any biomarker among phenotype groups compared were considered statistically significant where the FDR was less than 0.05. Examples of features include relative abundance (or normalized relative abundance), concentration (or normalized concentration), and site occupancy (fractional abundance across all glycans observed at the corresponding linking site of the corresponding peptide sequence).

For supervised multivariate modeling, a total of 1084 features (571 concentration, 49 relative abundance, and 464 site occupancy features) were log-transformed and split into a training set formed by 80% of all samples from women with benign pelvic tumors and EOC, and a testing set formed by the remaining 20% of these women and all healthy controls. To perform binary classification and predict the probability of EOC, repeated five-fold cross-validated LASSO-regularized logistic regression was used with hyperparameters tuned to prevent overfitting and promote balanced sensitivity and specificity metrics. Training of the binary classification model was performed using the subset of the 1084 total features having low coefficients of variation (<20%) in pooled serum replicates. This subset included 976 features, with each feature being a concentration, relative abundance, or site occupancy for a corresponding peptide structure and where some peptide structures correspond with multiple features. For example, a given peptide structure may be associated with one, two, or three features within the subset of the 976 features.

### VIII.A.4. Results

Normalized abundances of 428 peptide structures were found to display statistically significantly different abundances (FDR<0.05) in samples of patients with benign pelvic tumors and samples of patients with EOC. 139 peptide structures had statistically significant abundance differences between benign vs. early stage (e.g., stage 1 or 2) EOC. 412 peptide structures had statistically significant abundance differences between benign vs. late stage (e.g., stage 3 or 4) EOC, 137 of which overlapped with those for benign v. early stage. When comparing samples of healthy controls with samples from all EOCs, benign tumors, early stage (e.g., stage1 or 2) EOC, and late stage (e.g., stage 3 or 4) EOC, statistically significant abundances were found for 386, 149, 215, and 365 markers, respectively. 120 peptide structures were found to be statistically significantly differentially abundant in healthy controls vs. patients with benign pelvic tumors, and in healthy control vs. EOC. 200 peptide structures were found to be statistically significantly differentially abundant in in healthy control vs. early stage EOC and healthy control vs. late stage EOC. Lastly, of the 428 and 386 markers that were found statistically significantly differentially expressed between EOC vs. benign pelvic tumors and EOC vs. healthy controls, respectively, 328 were shared.

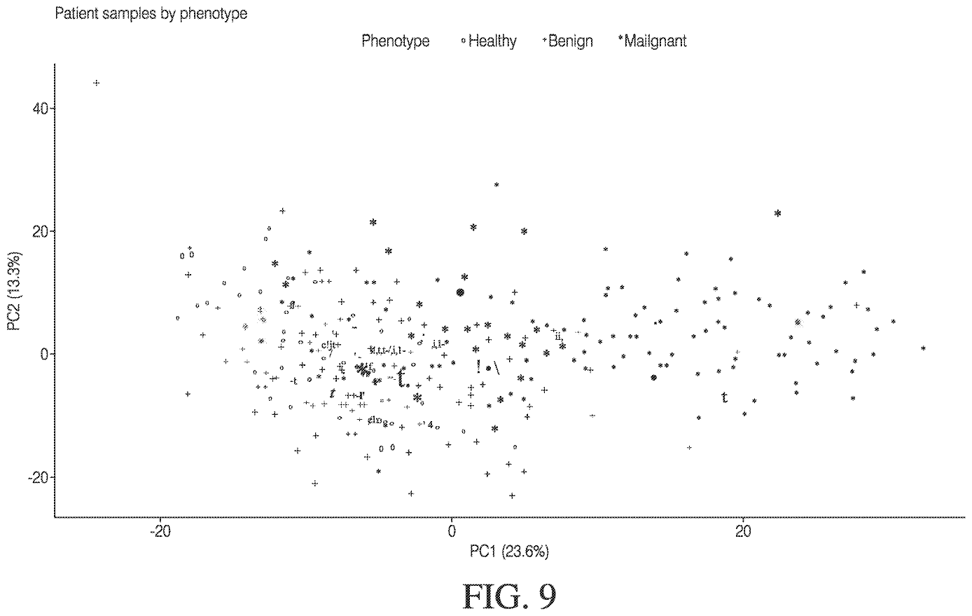

FIG. 9 is a plot diagram illustrating the results of a principal component analysis performed to assess the segregation between healthy, benign pelvic tumor, and EOC samples across first and second principal components in accordance with one or more embodiments. Generally, EOC samples segregated distinctly from healthy control samples, while most benign pelvic tumors did not segregate as distinctly from healthy control samples.

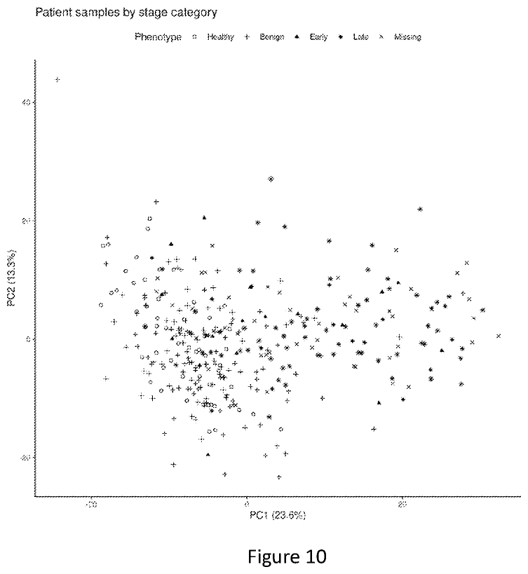

FIG. 10 is a plot diagram illustrating the results of a principal component analysis performed to assess segregation between healthy, benign pelvic tumor, early EOC, late EOC, and missing (undocumented) samples). Generally, EOC samples (and in particular late stage EOC samples) segregated distinctly from healthy control samples, while most benign pelvic tumors did not segregate as distinctly from healthy control samples.

### VIII.A.5. Results in Context of Screening for Malignant EOC

To assess the suitability of serum glycoproteomics in the context of screening for malignant EOC, a multivariable model was built to predict EOC vs. healthy status. This multivariable model is a supervised machine learning model that includes a logistic regression model, the logistic regression model including a LASSO regression model. Repeated cross-validation in the training set established the optimal LASSO hyperparameter (lambda=0.0608, cross-validated F1=0.971). Applying this amount of shrinkage to the panel of 976 features resulted in a logistic model with 10 peptide structures with non-zero coefficients.

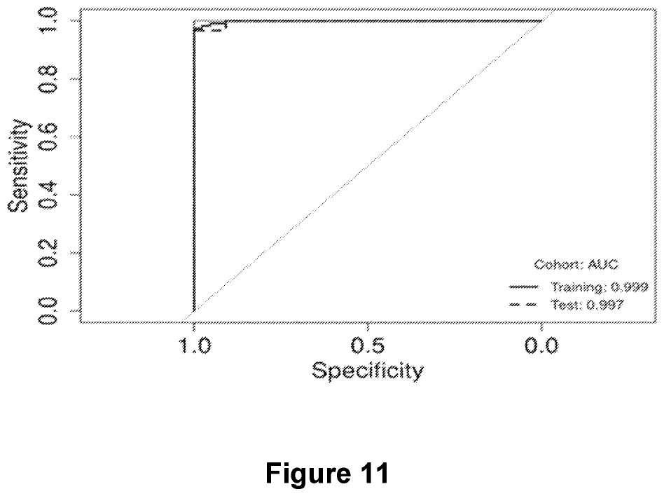

FIG. 11 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.975, sensitivity=0.983, specificity=0.955) and the test set (accuracy=0.976, sensitivity=0.967, specificity=1.0). Further, ROC analysis demonstrated strong performance across a range of cutoffs, and little overfitting, with the training AUC (area under the curve)=0.999 and test AUC=0.997.

Thus, the multivariable model that was built may be used accurately and reliably to malignant EOC and distinguish such malignancy from a healthy status. Such diagnostic power may be used to reduce the need for unnecessary invasive testing. Further, such diagnostic information can be used to identify patients with EOC earlier, which may lead to earlier treatment, improved treatment recommendations, and improved treatment plans.

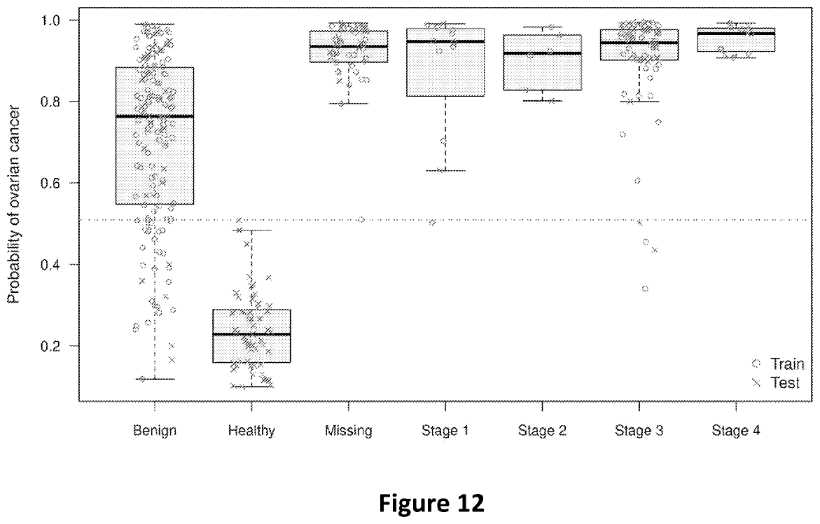

FIG. 12 is an illustration of a diagram showing the probability distributions for the various groups using the multivariable model for predicting malignancy v. benign status of pelvic tumors in accordance with one or more embodiments. As shown in FIG. 12, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the solidly predicts malignancy and severity of disease.

Table 8 below provides the fold changes, FDRs, and p-values for the 10 peptide structures PS-1 to PS-10 (same as those in Table 1 above) based on differential expression analysis (DEA). The peptide structures PS-1 to PS-10 are ordered both in Table 1 and in Table 8 with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 8
Peptide Structure Markers for Regression Model to distinguish
between Epithelial Ovarian Cancer and Healthy State

| | | Healthy v | Healthy | Healthy v | |
|---|---|---|---|---|---|
| PS-ID | | EOC (Fold | v EOC | EOC | |
| NO. | PS-NAME | Change) | (FDR) | (p-value) | Feature |
| PS-1 | ZA2G_128_5402 | 1.57212 | 1.99E−13 | 3.14E−15 | relab |
| PS-2 | IC1_253_6503 | 2.26917 | 6.42E−18 | 2.25E−20 | conc |
| PS-3 | CFAI_494_5402 | 1.30391 | 3.00E−07 | 4.78E−08 | relab |
| PS-4 | CERU_138_6513 | 1.37235 | 2.14E−06 | 4.85E−07 | relab |
| PS-5 | IGG1_297_3410 | 1.98807 | 1.03E−09 | 6.47E−11 | conc |
| PS-6 | HEMO_64_5402 | 1.53316 | 3.06E−11 | 1.12E−12 | relab |
| PS-7 | APOB_983_5402 | 1.98566 | 1.11E−13 | 1.17E−15 | conc |
| CK-1 | FINC_SYTITGL | 0.51932 | 9.92e−09 | 1.043e−09 | relab |
| | QPGTDYK | | | | |
| PS-8 | HPT_207_121005 | 2.21826 | 3.17E−10 | 1.66E−11 | conc |
| PS-9 | IGG3_297_3400 | N/A | N/A | N/A | relab |
| PS-10 | IGG4_297_3400 | N/A | N/A | N/A | relab |
| CK-2 | APOM_135_ | 0.59098 | 1.58e−17 | 8.28e−20 | conc |
| | 8500_CHK | | | | |

### VIII.A.6. Results in Context of Triaging Pelvic Tumors

To assess the suitability of serum glycoproteomics in the context of clinically triaging pelvic tumors, a multivariable model was built to predict malignancy vs. benign status of such pelvic tumors. This multivariable model is a supervised machine learning model that includes a logistic regression model, the logistic regression model including a LASSO regression model. Repeated cross-validation in the training set established the optimal LASSO hyperparameter (lambda=0.045, cross-validated F1=0.849). Applying this amount of shrinkage to the panel of 976 features resulted in a logistic model with 25 peptide structures with non-zero coefficients.

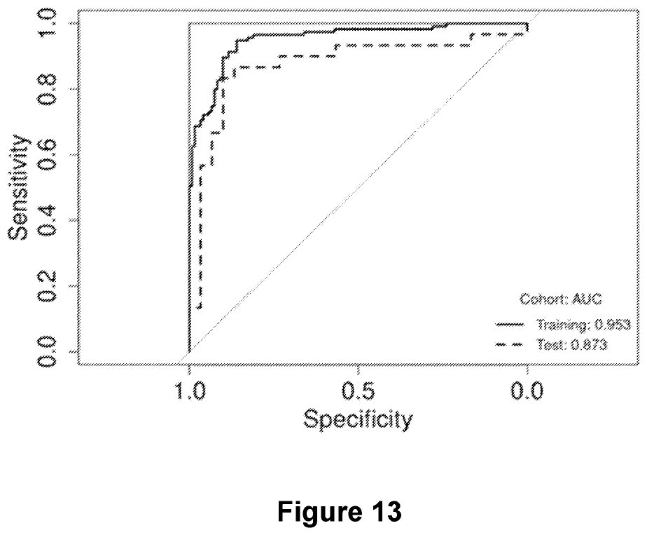

FIG. 13 is an illustration of a receiver operating characteristic (ROC) diagram corresponding to the multivariable model built to predict malignancy v. benign status of pelvic tumors in accordance with one or more embodiments. The multivariable model achieved high accuracy in both the training set (accuracy=0.869, sensitivity=0.835, specificity=0.901) and the test set (accuracy=0.867, sensitivity=0.867, specificity=0.867). Further, ROC analysis demonstrated strong performance across a range of cutoffs, and little overfitting, with the training AUC (area under the curve)=0.953 and test AUC=0.873.

Thus, the multivariable model that was built may be used accurately and reliably to triage pelvic tumors and distinguish those that are malignant from those that are benign. Such diagnostic power may be used to reduce the need for invasive testing (e.g., biopsy) prior to treatment can be administered. Further, such diagnostic information can be used to improve treatment recommendations and treatment plans (e.g., earlier treatment in the case of malignant EOC) and reduce indications for unnecessary treatment (e.g., no indication for surgery when the pelvic tumor is benign).

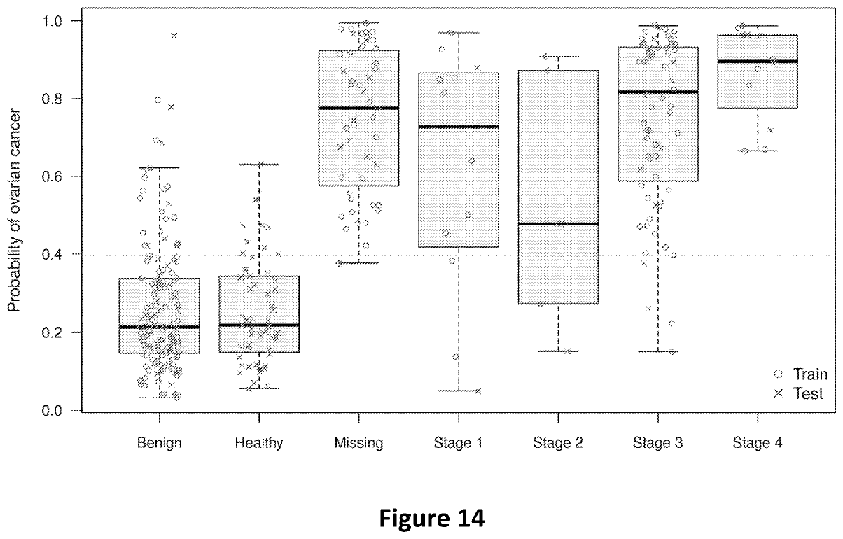

FIG. 14 is an illustration of a diagram showing the probability distributions for the various groups using the multivariable model for predicting malignancy v. benign status of pelvic tumors in accordance with one or more embodiments. As shown in FIG. 12, the probability distributions for benign pelvic tumor, healthy, missing (undocumented), stage 1 EOC, stage 2 EOC, stage 3 EOC, and stage 4 EOC samples increased with cancer stage, with probability distributions being similar across training and test sets. Notably, applying the built multivariable model to healthy patients, who were not utilized in the training, resulted in few misclassifications and a spread nearly equivalent to that of the benign pelvic tumor cases. Such results indicate that the glycoproteomic signature of the 25 peptide structures for the LASSO regression model solidly predict malignancy and severity of disease.

Table 9 below provides the fold changes, FDRs, and p-values for the 25 peptide structures PS-5 and PS-11 to PS-34 (same as those in Table 2 above) based on differential expression analysis (DEA). The peptide structures PS-5 and PS-11 to PS-34 are ordered both in Table 2 and in Table 9 with respect to relative significance to the probability score generated by the model. More significant peptide structures had higher coefficients in the LASSO regression model, while less significant peptide structures had lower coefficients in the LASSO regression model. In other words, relative significance to the probability score decreased with decreasing coefficients. Further, each peptide structure is associated with a feature that was used for the model (relab=relative abundance; conc=concentration).

TABLE 9
Peptide Structure Markers for Regression Model to distinguish
between Epithelial Ovarian Cancer and Benign Pelvic Tumor

| | | Benign | Benign | | |
|---|---|---|---|---|---|
| | | v. EOC | v. | Benign v. | |
| PS-ID | | (Fold | EOC | EOC | |
| NO. | PS-NAME | Change) | (FDR) | (p-value) | Feature |
| CK-3 | APOD_98_9800 | 1.54848 | 4.78e−13 | 8.46e−14 | relab |
| | CHECK | | | | |
| PS-11 | CO2_621_5200 | 1.36880 | 1.73E−11 | 3.66E−12 | relab |
| PS-5 | IGG1_297_3410 | 1.54336 | 2.47E−10 | 6.61E−11 | relab |
| PS-12 | AGP1_93_7612 | 2.39546 | 2.79E−16 | 2.20E−17 | relab |
| PS-13 | AACT_271_7602 | 1.68006 | 2.27E−08 | 7.70E−09 | conc |
| PS-14 | A2MG_1424_5402 | 1.15594 | 0.007733584 | 0.005106062 | relab |
| PS-15 | AACT_271_6513 | 2.34075 | 2.81E−18 | 1.04E−19 | relab |
| PS-16 | CERU_397_5402 | 1.07300 | 0.008195667 | 0.005425503 | relab |
| PS-17 | APOB_3411_5301 | 1.01808 | 0.743228938 | 0.714593147 | relab |
| PS-18 | AACT_106_6513 | 2.11211 | 1.42E−16 | 9.67E−18 | relab |
| PS-19 | CERU_138_5402 | 1.08927 | 0.002831028 | 0.001760096 | conc |
| PS-20 | A1AT_107_6513 | 2.15635 | 6.82E−14 | 1.06E−14 | relab |
| PS-21 | AGP1_93_7602 | 1.11780 | 0.012740002 | 0.008679266 | relab |
| PS-22 | VTNC_242_6502 | 0.83257 | 0.000446981 | 0.000252845 | relab |
| PS-23 | IGG2_297_3510 | 0.69463 | 8.28E−10 | 2.36E−10 | conc |
| PS-24 | CFAH_882_5411 | 0.84102 | 1.06E−05 | 4.78E−06 | relab |
| CK-4 | APOM_135_8500_ | 0.81884 | 1.16e−08 | 3.87e−09 | conc |
| | CHECK | | | | |
| PS-25 | AGP1_103_8704 | 1.18615 | 0.001152856 | 0.000676369 | relab |
| PS-26 | IGG1_297_4300 | 0.60088 | 2.09E−11 | 4.47E−12 | relab |
| PS-27 | APOH_253_5401 | 0.62217 | 1.65E−16 | 1.16E−17 | conc |
| PS-28 | APOD_98_5411 | 0.71180 | 1.50E−12 | 2.82E−13 | conc |
| PS-29 | TRFE_630_5411 | 0.69298 | 4.01E−14 | 5.62E−15 | conc |
| PS-30 | CERU_138_6502 | 0.81476 | 7.13E−07 | 2.87E−07 | relab |
| PS-31 | A2MG_1424_5411 | 0.67638 | 1.53E−23 | 2.68E−26 | conc |
| PS-32 | A2MG_55_5411 | 0.71212 | 2.20E−20 | 1.93E−22 | conc |
| PS-33 | TRFE_630_5412 | 0.77453 | 1.01E−09 | 2.95E−10 | conc |
| PS-34 | IGG2_297_4511 | 0.73039 | 3.50E−08 | 1.23E−08 | conc |

Table 10 below provides the fold changes, FDRs, and p-values for the 36 peptide structures PS-4, PS-8, PS-18, PS-36, PS-37, PS-41, PS-56, PS-62 to PS-90 (same as those in Table 3B above) based on differential expression analysis (DEA). The peptide structures PS-4, PS-8, PS-18, PS-36, PS-37, PS-41, PS-56, PS-62 to PS-90 are ordered in Table 10 with respect to relative significance to the p value score generated by the model.

TABLE 10
Peptide Structure Markers for Regression Model to distinguish
between late stage (3/4) EOC and early stage EOC (1/2)
using the biomarkers of Table 3B.

| | | Benign v. | | |
|---|---|---|---|---|
| | | EOC | Benign v. | Benign |
| PS-ID | | (Fold | EOC | v. EOC |
| NO. | PS-NAME | Change) | (FDR) | (p-value) |
| PS-66 | AGP1_103_6513 | 1.539 | 0.0001 | 1.22E−06 |
| PS-89 | KNG1_294_6503 | 0.817 | 0.0006 | 1.94E−05 |
| PS-41 | TRFE_630_6513 | 1.361 | 0.001 | 3.71E−05 |
| PS-85 | HPT_207_11915 | 1.469 | 0.0011 | 4.62E−05 |
| PS-68 | AGP1_103_7614 | 1.425 | 0.0011 | 5.35E−05 |
| PS-8 | HPT_207_121005 | 0.736 | 0.0013 | 7.32E−05 |
| PS-83 | HPT_184_6513 | 1.575 | 0.002 | 0.0001 |
| PS-86 | HPT_241_6513 | 1.64 | 0.0022 | 0.0002 |
| PS-88 | KNG1_205_6513 | 1.512 | 0.0028 | 0.0002 |
| PS-71 | AGP1_93_7613 | 1.436 | 0.0031 | 0.0003 |
| PS-72 | AGP12_56_6503 | 0.78 | 0.0031 | 0.0003 |
| PS-74 | AGP12_72_6503 | 0.759 | 0.0034 | 0.0003 |
| PS-81 | HEMO_187_6503 | 0.861 | 0.0034 | 0.0003 |
| PS-77 | APOH_162_6503 | 0.799 | 0.0035 | 0.0003 |
| PS-87 | HPT_241_7613 | 1.58 | 0.0039 | 0.0004 |
| PS-70 | AGP1_93_6513 | 1.398 | 0.0039 | 0.0004 |
| PS-79 | CERU_762_6513 | 1.19 | 0.0039 | 0.0004 |
| PS-37 | AGP1_93_7614 | 1.327 | 0.0039 | 0.0004 |
| PS-62 | AACT_106_6503 | 0.749 | 0.0045 | 0.0005 |
| PS-65 | AGP1_103_6503 | 0.792 | 0.0045 | 0.0006 |
| PS-80 | FETUA_156_6503 | 0.837 | 0.0045 | 0.0006 |
| PS-64 | AACT_271_6502 | 0.688 | 0.0046 | 0.0006 |
| PS-90 | VTNC_242_6503 | 0.836 | 0.0049 | 0.0007 |
| PS-67 | AGP1_103_7602 | 0.767 | 0.0049 | 0.0007 |
| PS-56 | AACT_106_7614 | 1.379 | 0.0051 | 0.0007 |
| PS-76 | AGP2_103_6513 | 1.438 | 0.0053 | 0.0008 |
| PS-63 | AACT_106_7604 | 0.794 | 0.0056 | 0.0009 |
| PS-73 | AGP12_56_6513 | 1.426 | 0.0065 | 0.001 |
| PS-18 | AACT_106_6513 | 1.336 | 0.0096 | 0.0017 |
| PS-82 | HEMO_187_6513 | 1.232 | 0.0107 | 0.002 |
| PS-69 | AGP1_93_6503 | 0.796 | 0.0112 | 0.0022 |
| PS-36 | FETUA_176_6513 | 1.394 | 0.0123 | 0.0025 |
| PS-4 | CERU_138_6513 | 1.143 | 0.0253 | 0.0066 |
| PS-75 | AGP12_72_7603 | 0.835 | 0.0259 | 0.0069 |
| PS-78 | CERU_397_6513 | 1.225 | 0.0375 | 0.0113 |
| PS-84 | HPT_207_11904 | 0.865 | 0.0386 | 0.0117 |

Table 10B below provides the fold changes, FDRs, and p-values for the 25 peptide structures denoted by SEQ ID NO 101-125 (in accordance with Table 3C above) using differential expression analysis (DEA).

TABLE 10B
Peptide Structure Markers for Regression Model to distinguish
between late stage (3/4) EOC and early stage
EOC (1/2) using the biomarkers of Table 3C.

| | | stage 3, 4 vs | stage | stage |
|---|---|---|---|---|
| SEQ | | stage 1, 2 | 3, 4 vs | 3, 4 vs |
| ID | | (fold_ | stage 1, 2 | stage 1, 2 |
| NO | PS-NAME | change) | (FDR) | (p-value) |
| 101 | AACT_271_6512 | 0.704 | 0.0028 | 0.0002 |
| 102 | AGP1_103_6503 | 0.792 | 0.0045 | 0.0006 |
| 103 | AGP1_103_6513 | 1.539 | 0.0001 | 1.22E−06 |
| 104 | AGP1_103_7614 | 1.425 | 0.0011 | 5.35E−05 |
| 105 | AGP1_93_6513 | 1.398 | 0.0039 | 0.0004 |
| 106 | AGP1_93_7604 | 1.471 | 0.001 | 3.94E−05 |
| 107 | AGP1_93_7613 | 1.436 | 0.0031 | 0.0003 |
| 108 | AGP1_93_7614 | 1.327 | 0.0039 | 0.0004 |
| 109 | AGP12_56_6503 | 0.78 | 0.0031 | 0.0003 |
| 110 | AGP12_72_6503 | 0.759 | 0.0034 | 0.0003 |
| 111 | AGP12_72_7604 | 1.546 | 0.0012 | 6.85E−05 |
| 112 | AGP12_72MC_7604 | 1.592 | 0.0013 | 8.30E−05 |
| 113 | AGP12_72MC_7614 | 1.464 | 0.002 | 0.0001 |
| 114 | APOH_162_6503 | 0.799 | 0.0035 | 0.0003 |
| 115 | CERU_762_6513 | 1.19 | 0.0039 | 0.0004 |
| 116 | HEMO_187_6503 | 0.861 | 0.0034 | 0.0003 |
| 117 | HPT_184_6513 | 1.575 | 0.002 | 0.0001 |
| 118 | HPT_207_11915 | 1.469 | 0.0011 | 4.62E−05 |
| 119 | HPT_207_121005 | 0.736 | 0.0013 | 7.32E−05 |
| 120 | HPT_241_6503 | 1.607 | 0.001 | 4.78E−05 |
| 121 | HPT_241_6513 | 1.64 | 0.0022 | 0.0002 |
| 122 | HPT_241_7613 | 1.58 | 0.0039 | 0.0004 |
| 123 | KNG1_205_6513 | 1.512 | 0.0028 | 0.0002 |
| 124 | KNG1_294_6503 | 0.817 | 0.0006 | 1.94E−05 |
| 125 | TRFE_630_6513 | 1.361 | 0.001 | 3.71E−05 |

Table 10C below provides the fold changes, FDRs, and p-values for the 50 peptide structures denoted by SEQ ID NO 126-175 (in accordance with Table 3D above) using differential expression analysis (DEA). For this differential expression analysis, the subjects from EOC stages 1-4 were used for this analysis as shown in the table of FIG. 8. EOC stages 1 and 2 (12+6) were combined to be the early stage cohort and EOC stages 3 and 4 (68+12) were combined to be the late stage cohort.

TABLE 10C
Peptide Structure Markers for Regression Model to
distinguish between late stage (3/4) EOC and early stage
EOC (1/2) using the biomarkers of Table 3D.

| | | stage 3, 4 vs | stage | stage |
|---|---|---|---|---|
| SEQ | | stage 1, 2 | 3, 4 vs | 3, 4 vs |
| ID | | (fold_ | stage 1, 2 | stage 1, 2 |
| NO | PS-Name | change) | (FDR) | (p-value) |
| 126 | A1AT_107_6512 | 1.319 | 0.294 | 0.454 |
| 127 | A2MG_1424_6501 | 1.248 | 0.129 | 0.274 |
| 128 | A2MG_1424_6511 | 1.025 | 0.776 | 0.868 |
| 129 | AACT_106_6503 | 0.739 | 0.035 | 0.132 |
| 130 | AACT_106_7604 | 0.789 | 0.040 | 0.148 |
| 131 | AACT_106_7614 | 1.319 | 013 | 0.076 |
| 132 | AACT_271_6502 | 0.587 | 0.004 | 0.056 |
| 133 | AACT_271_6503 | 0.663 | 0.013 | 0.076 |
| 134 | AGP12_56_6503 | 0.746 | 0.005 | 0.059 |
| 135 | AGP12_72MC_6503 | 0.841 | 0.099 | 0.239 |
| 136 | AGP12_72MC_6513 | 1.389 | 0.003 | 0.054 |
| 137 | AGP12_72MC_7613 | 1.561 | 0.001 | 0.035 |
| 138 | AGP12_72MC_7614 | 1.669 | 0.001 | 0.035 |
| 139 | AGP12_72_6503 | 0.685 | 0.005 | 0.061 |
| 140 | AGP12_72_7603 | 0.769 | 0.027 | 0.115 |
| 141 | AGP12_72_7604 | 0.835 | 0.083 | 0.22 |
| 142 | AGP1_103_6503 | 0.838 | 0.125 | 0.269 |
| 143 | AGP1_103_6513 | 1.481 | <0.001 | 0.013 |
| 144 | AGP1_103_7602 | 0.705 | 0.013 | 0.076 |
| 145 | AGP1_103_7604 | 0.866 | 0.245 | 0.408 |
| 146 | AGP1_103_7614 | 1.386 | <0.001 | 0.013 |
| 147 | AGP1_93_6503 | 0.828 | 0.125 | 0.269 |
| 148 | AGP1_93_7602 | 0.854 | 0.182 | 0.3417 |
| 149 | AGP1_93_7603 | 0.887 | 0.243 | 0.404 |
| 150 | AGP1_93_7604 | 0.951 | 0.624 | 0.761 |
| 151 | AGP1_93_7612 | 1.293 | 0.031 | 0.126 |
| 152 | AGP1_93_7613 | 1.417 | 0.001 | 0.03 |
| 153 | AGP1_93_7614 | 1.508 | <0.001 | 0.015 |
| 154 | AGP2_103_6503 | 0.805 | 0.094 | 0.232 |
| 155 | AGP2_103_6513 | 1.386 | <0.001 | 0.015 |
| 156 | AGP2_103_7604 | 0.836 | 0.173 | 0.334 |
| 157 | APOH_162_6503 | 0.856 | 0.099 | 0.239 |
| 158 | APOH_253_5412 | 1.278 | 0.0185 | 0.093 |
| 159 | CERU_397_6513 | 1.323 | 0.012 | 0.076 |
| 160 | CERU_762_6503 | 0.814 | 0.019 | 0.095 |
| 161 | FETUA_156_6503 | 0.856 | 0.043 | 0.155 |
| 162 | FETUA_156_6513 | 1.155 | 0.222 | 0.387 |
| 163 | FETUA_176_6513 | 1.358 | 0.088 | 0.228 |
| 164 | HEMO_187_6503 | 0.882 | 0.056 | 0.175 |
| 165 | HEMO_187_6513 | 1.214 | 0.044 | 0.155 |
| 166 | HPT_184_6513 | 1.50 | 0.032 | 0.130 |
| 167 | HPT_207_11904 | 0.812 | 0.038 | 0.141 |
| 168 | HPT_207_11914 | 1.253 | 0.0128 | 0.076 |
| 169 | HPT_207_11915 | 1.55 | <0.001 | 0.015 |
| 170 | HPT_207_121005 | 0.742 | 0.0136 | 0.077 |
| 171 | HPT_241_6511 | 1.217 | 0.171 | 0.33 |
| 172 | HPT_241_6512 | 1.595 | 0.01 | 0.07 |
| 173 | HPT_241_6513 | 1.629 | 0.007 | 0.065 |
| 174 | HPT_241_7613 | 1.506 | 0.053 | 0.172 |
| 175 | KNG1_294_6503 | 0.8 | 0.003 | 0.054 |

Table 10D below provides the fold changes, FDRs, and p-values for the 12 peptide structures denoted by SEQ ID NO 131-134, 137, 139, 140, 143, 151, 165-167 (in accordance with Table 3D above) using differential expression analysis (DEA). For this differential expression analysis, the subjects from EOC stages 1-4 were used for this analysis as shown in the table of FIG. 8. EOC stages 1 and 2 (12+6) were combined to be the early stage cohort and EOC stages 3 and 4 (68+12) were combined to be the late stage cohort.

TABLE 10D
Peptide Structure Markers for Regression Model to distinguish
between late stage (3/4) EOC and early stage EOC (1/2)
using 12 of the biomarkers of Table 3D.

| | | stage 3, 4 vs | stage | stage |
|---|---|---|---|---|
| SEQ | | stage 1, 2 | 3, 4 vs | 3, 4 vs |
| ID | | (fold_ | stage 1, 2 | stage 1, 2 |
| NO | PS-Name | change) | (FDR) | (p-value) |
| 131 | AACT_106_7614 | 1.319 | 0.0131 | 0.07632 |
| 132 | AACT_271_6502 | 0.587 | 0.00394 | 0.05633 |
| 133 | AACT_271_6503 | 0.663 | 0.0116 | 0.07632 |
| 134 | AGP12_56_6503 | 0.746 | 0.00511 | 0.05958 |
| 137 | AGP12_72MC_7613 | 1.561 | 0.00139 | 0.03491 |
| 139 | AGP12_72_6503 | 0.685 | 0.00539 | 0.06138 |
| 140 | AGP12_72_7603 | 0.769 | 0.0269 | 0.11519 |
| 143 | AGP1_103_6513 | 1.481 | 0.00013 | 0.0134 |
| 151 | AGP1_93_7612 | 1.294 | 0.03077 | 0.12634 |
| 165 | HEMO_187_6513 | 1.214 | 0.04408 | 0.15552 |
| 166 | HPT_184_6513 | 1.501 | 0.03264 | 0.13004 |
| 167 | HPT_207_11904 | 0.813 | 0.03824 | 0.14089 |

The markers from Table 3B were used to train a regularized regression model (e.g., LASSO regression model). Coefficients for the regularized regression model (e.g., LASSO regression model) are provided in Table 11. Using the values of Table 11, a probability for one of the states can be determined by summing together the product of the concentration of each biomarker in the sample and the respective coefficient (of one column) and then adding the summation and the intercept to yield the logit of a probability score. For example, the logit of the probability, to which the inverse logit function can be applied, is equal to the following equation 1 (eq. 1).

$${\sum }_{i = 1}^{i = n} [ ( {Concentration}_{PS - ID NO : i} ) x ( {Coefficient}_{PS - ID NO : i} ) ] + Intercept \qquad ( eq . 1 )$$

where n=a number of biomarkers having a unique PS-ID No,
i=an index number for each of the biomarkers,

TABLE 11
Coefficients for Peptide Structure Markers for a Regression
Model to distinguish between late stage (3/4) EOC and early
stage EOC (1/2) based on the biomarkers of Table 3B

| | | Coefficients |
|---|---|---|
| | | (stage 3, 4 |
| | | vs stage |
| PS-ID No | PS-NAME | 1, 2) |
| PS-62 | AACT_106_6503 | 0.789963 |
| PS-18 | AACT_106_6513 | −0.100283 |
| PS-63 | AACT_106_7604 | −0.23972 |
| PS-56 | AACT_106_7614 | −0.174242 |
| PS-64 | AACT_271_6502 | −1.019336 |
| PS-65 | AGP1_103_6503 | −1.129647 |
| PS-66 | AGP1_103_6513 | 2.450196 |
| PS-67 | AGP1_103_7602 | −0.826335 |
| PS-68 | AGP1_103_7614 | 0.342801 |
| PS-69 | AGP1_93_6503 | 0.888268 |
| PS-70 | AGP1_93_6513 | −1.638508 |
| PS-71 | AGP1_93_7613 | −0.120831 |
| PS-37 | AGP1_93_7614 | −0.94578 |
| PS-72 | AGP12_56_6503 | −0.211526 |
| PS-73 | AGP12_56_6513 | 0.105946 |
| PS-74 | AGP12_72_6503 | −1.586863 |
| PS-75 | AGP12_72_7603 | 0.544458 |
| PS-76 | AGP2_103_6513 | −0.132156 |
| PS-77 | APOH_162_6503 | 0.465722 |
| PS-4 | CERU_138_6513 | |
| PS-78 | CERU_397_6513 | −1.056969 |
| PS-79 | CERU_762_6513 | 1.538329 |
| PS-80 | FETUA_156_6503 | −0.478039 |
| PS-36 | FETUA_176_6513 | 0.343385 |
| PS-81 | HEMO_187_6503 | −0.012419 |
| PS-82 | HEMO_187_6513 | −2.077228 |
| PS-83 | HPT_184_6513 | 1.018212 |
| PS-84 | HPT_207_11904 | 0.950984 |
| PS-85 | HPT_207_11915 | 1.289189 |
| PS-8 | HPT_207_121005 | −0.387854 |
| PS-86 | HPT_241_6513 | −0.890611 |
| PS-87 | HPT_241_7613 | 0.311607 |
| PS-88 | KNG1_205_6513 | 0.287132 |
| PS-89 | KNG1_294_6503 | 0.188466 |
| PS-41 | TRFE_630_6513 | −0.19935 |
| PS-90 | VTNC_242_6503 | −0.11461 |

The markers from Table 3C were used to train a regularized regression model (e.g., LASSO regression model). Coefficients for the regularized regression model (e.g., LASSO regression model) are provided in Table 11B. Using the values of Table 11B, a probability for one of the states can be determined by summing together the product of the concentration of each biomarker in the sample and the respective coefficient (of one column) and then adding the summation and the intercept to yield the logit of a probability score (see equation 1).

TABLE 11B
Coefficients for Peptide Structure Markers for a Regression
Model to distinguish between late stage (3/4) EOC and early
stage EOC (1/2) based on the biomarkers of Table 3C

| SEQ ID NO | PS-NAME | Coefficients |
|---|---|---|
| 101 | AACT_271_6512 | 0.267422 |
| 102 | AGP1_103_6503 | −0.44212 |
| 103 | AGP1_103_6513 | 1.735177 |
| 104 | AGP1_103_7614 | 0.240767 |
| 105 | AGP1_93_6513 | −1.34581 |
| 106 | AGP1_93_7604 | 0.346537 |
| 107 | AGP1_93_7613 | −0.33861 |
| 108 | AGP1_93_7614 | −1.4923 |
| 109 | AGP12_56_6503 | −0.75867 |
| 110 | AGP12_72_6503 | −0.57017 |
| 111 | AGP12_72_7604 | 0.214311 |
| 112 | AGP12_72MC_7604 | −0.42256 |
| 113 | AGP12_72MC_7614 | 0.675864 |
| 114 | APOH_162_6503 | 0.124674 |
| 115 | CERU_762_6513 | 0.180667 |
| 116 | HEMO_187_6503 | −0.35174 |
| 117 | HPT_184_6513 | 0.895905 |
| 118 | HPT_207_11915 | 0.362611 |
| 119 | HPT_207_121005 | −0.17095 |
| 120 | HPT_241_6503 | 0.26564 |
| 121 | HPT_241_6513 | −2.44213 |
| 122 | HPT_241_7613 | −0.0651 |
| 123 | KNG1_205_6513 | 0.318803 |
| 124 | KNG1_294_6503 | −0.02677 |
| 125 | TRFE_630_6513 | 0.782865 |

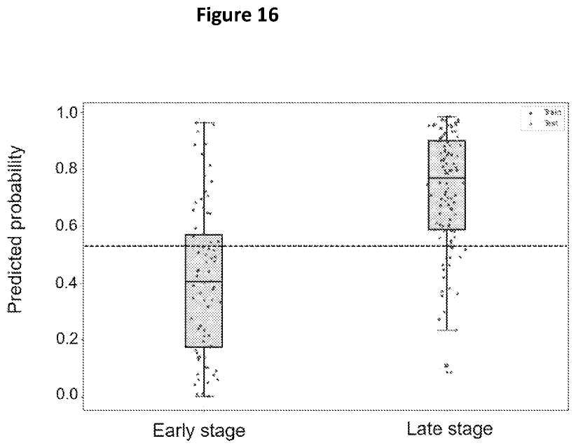

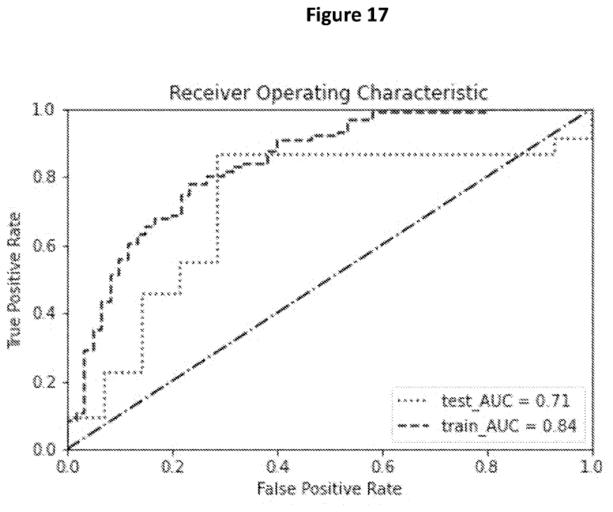

Using the model coefficients of Table 11B, a predicted probability was generated for early stage and late stage ovarian cancer samples showing a stratification in predicted probabilities between the two cohorts as is illustrated in FIG. 16. FIG. 17 illustrates a receiver-operating-characteristic (ROC) curve and the area under curve (AUC) for the regularized regression model (e.g., LASSO regression model) for early stage and late stage ovarian cancer samples using testing case data and training case data. Table 12 shows the accuracy, sensitivity, specificity and precision for the training data set and the testing data set. Table 13 shows the training accuracy and testing accuracy for the early stage and late stage cohort for ovarian cancer.

TABLE 12

| | Train | Test |
|---|---|---|
| Accuracy | 0.753 | 0.778 |
| Sensitivity | 0.802 | 0.864 |
| Specificity | 0.7 | 0.714 |
| Precision | 0.712 | 0.769 |

TABLE 13

| | Train | Test |
|---|---|---|
| | (accuracy) | (accuracy) |
| Early Stage | 0.7 | 0.714286 |
| Late Stage | 0.802326 | 0.863636 |

The markers from Table 3D were used to train a regularized regression model (e.g., LASSO regression model). Coefficients for the regularized regression model (e.g., LASSO regression model) are provided in Table 11C. Using the values of Table 11C, a probability for one of the states can be determined by summing together the product of the concentration of each biomarker in the sample and the respective coefficient (of one column) and then adding the summation and the intercept to yield the logit of a probability score (see equation 1).

TABLE 11C
Coefficients for Peptide Structure Markers for a Regression
Model to distinguish between late stage (3/4) EOC and
early stage EOC (1/2) based on the biomarkers of Table 3D

| SEQ ID NO | PS-Name | Coefficients |
|---|---|---|
| 126 | A1AT_107_6512 | 0 |
| 127 | A2MG_1424_6501 | 0 |
| 128 | A2MG_1424_6511 | 0 |
| 129 | AACT_106_6503 | 0 |
| 130 | AACT_106_7604 | −0.0022402 |
| 131 | AACT_106_7614 | −0.652235354 |
| 132 | AACT_271_6502 | −0.680231822 |
| 133 | AACT_271_6503 | −1.169551 |
| 134 | AGP12_56_6503 | −2.2045339 |
| 135 | AGP12_72MC_6503 | 0.56949569 |
| 136 | AGP12_72MC_6513 | 0 |
| 137 | AGP12_72MC_7613 | −2.2203046 |
| 138 | AGP12_72MC_7614 | 0 |
| 139 | AGP12_72_6503 | −1.5094621 |
| 140 | AGP12_72_7603 | 0.91786725 |
| 141 | AGP12_72_7604 | 0 |
| 142 | AGP1_103_6503 | 0 |
| 143 | AGP1_103_6513 | 1.51746308 |
| 144 | AGP1_103_7602 | 0 |
| 145 | AGP1_103_7604 | 0 |
| 146 | AGP1_103_7614 | 0 |
| 147 | AGP1_93_6503 | 0 |
| 148 | AGP1_93_7602 | 0.05936161 |
| 149 | AGP1_93_7603 | 1.69720204 |
| 150 | AGP1_93_7604 | 0 |
| 151 | AGP1_93_7612 | 0 |
| 152 | AGP1_93_7613 | 0 |
| 153 | AGP1_93_7614 | 0 |
| 154 | AGP2_103_6503 | 0 |
| 155 | AGP2_103_6513 | 0.15270371 |
| 156 | AGP2_103_7604 | 0 |
| 157 | APOH_162_6503 | 0 |
| 158 | APOH_253_5412 | 0.84222935 |
| 159 | CERU_397_6513 | 0.07304956 |
| 160 | CERU_762_6503 | −0.106671 |
| 161 | FETUA_156_6503 | 0.50770712 |
| 162 | FETUA_156_6513 | −1.2528564 |
| 163 | FETUA_176_6513 | 0 |
| 164 | HEMO_187_6503 | 0 |
| 165 | HEMO_187_6513 | 0 |
| 166 | HPT_184_6513 | 0.25532311 |
| 167 | HPT_207_11904 | 0 |
| 168 | HPT_207_11914 | 0 |
| 169 | HPT_207_11915 | 0 |
| 170 | HPT_207_121005 | 0 |
| 171 | HPT_241_6511 | 0.50726531 |
| 172 | HPT_241_6512 | 0 |
| 173 | HPT_241_6513 | 0 |
| 174 | HPT_241_7613 | 0 |
| 175 | KNG1_294_6503 | 0 |

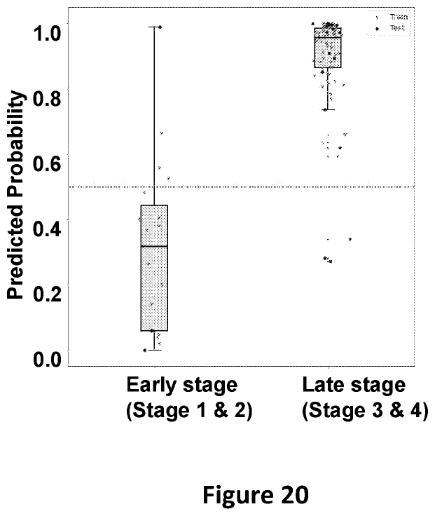

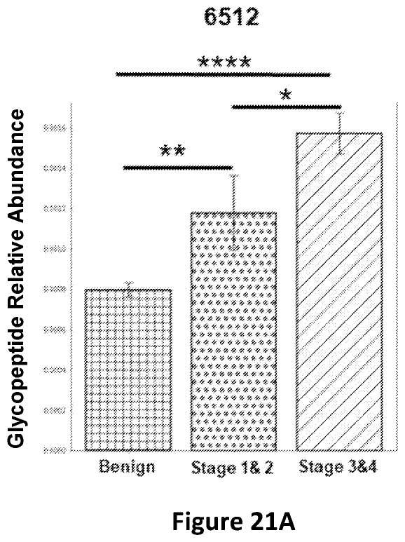

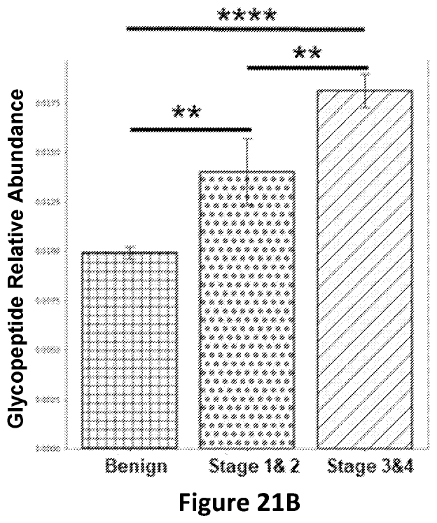

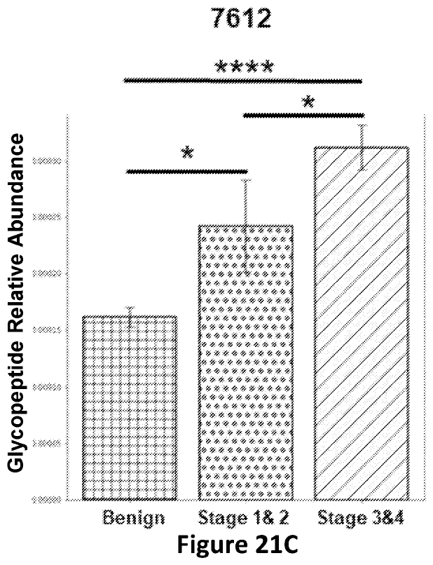

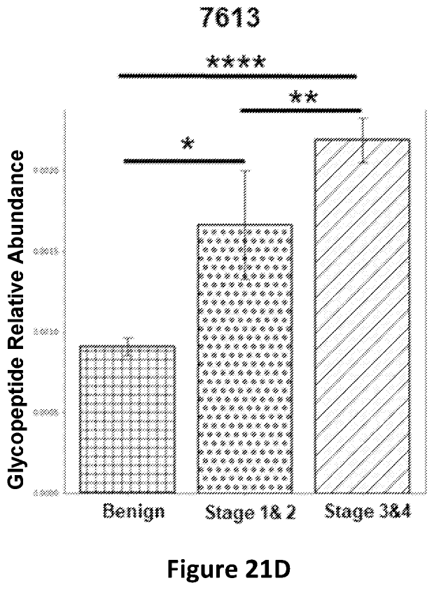

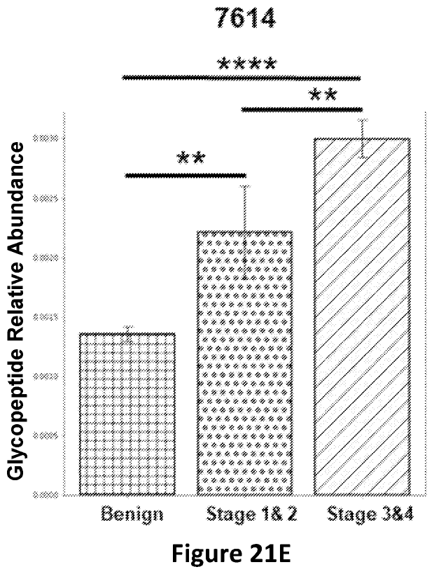

Using the model coefficients of Table 11C, a predicted probability was generated for early stage and late stage ovarian cancer samples showing a stratification in predicted probabilities between the two cohorts as is illustrated in FIG. 20. In various embodiments, predicted probability can be generated for classifying early stage and late stage ovarian cancer samples using the markers with non-zero coefficients such as SEQ ID NO's 130-135, 137, 139, 140, 143, 148, 149, 155, 158-162, 166, and 171. A logistic regression model was used with the glycopeptides of Table 3D where the glycopeptides had 1 or more sialic acids and zero or more fucosylations for the early and late stage EOC cohorts. The glycopeptides that included fucose were found to be associated with EOC. In addition, glycopeptides that included fucose and also carrying tri- and tetra-antennary glycan structure were found to be more strongly associated with EOC. For example, FIGS. 21A to 21E show that the relative abundance of tri- and tetra-antennary glycan structures in benign tumors, early-stage EOC and late-stage EOC showed an increase with the progression of the EOC disease. The numbers 6512, 6512, 7612, 7613, 7614 correspond to the five distinct glycans attached to the glycopeptides. Referring back to FIGS. 21A to 21C, the three leftmost bar graphs represent glycopeptides with tetra-antennary glycans with varying degrees of sialylation. The two rightmost bar graphs are FIGS. 21D and 21E and they represent glycopeptides with tri-antennary glycans with two or three sialic acids. The asterisk(s) in FIGS. 21A and 21E represent the following-*p-value<=0.05, **p-value<=0.01, ***p-value<=0.001, and ****p-value<=0.0001). It should be noted that the horizontal bars on FIGS. 21A to 21E are used to show the statistical comparisons between the benign and late-stage cohorts (highest horizontal bar), early-stage and late-stage cohorts (middle horizontal bar), and the benign and early-stage cohorts (lowest horizontal bar),

Table 14 shows the accuracy, sensitivity, and specificity for the training data set and the testing data set.

TABLE 14

| | Train | Test |
|---|---|---|
| Accuracy | 0.963 | 0.8 |
| Sensitivity | 1 | 0.824 |
| Specificity | 0.812 | 0.667 |

In various embodiments, a specific subset of tri- and tetra-antennary fucosylated N-glycopeptides were identified that can be used to differentiate between early- and late-stage ovarian cancer. In particular, the fucose portion of the specific subset of tri- and tetra-antennary fucosylated N-glycopeptides were found to have an outer arm position. It should be noted that fucose can be bound to a glycan in a core fucosylation or outer-arm orientation. Core fucosylation is a modification of a N-glycan core structure, forming the α1,6 fucosylation of the GlcNAc residue linked to the asparagine, that is catalyzed by FUT8. A fucose in the outer-arm orientation is attached to the antennae of the complex type N-glycans by α-(1-3/4) linkage to the GlcNAc residues or by α-(1-2) linkage to galactose.

To elucidate the site of fucosylation in these markers, high-resolution LC-MS/MS analysis was used. A subset of 25 samples from the original set (5 each of from individuals with benign, stage I, II, III, or IV tumors) were chosen for high resolution MS analysis using serum. Samples were digested with trypsin as described above and enriched using an Oasis MAX 96-well plate (30 μm particle size, 30 mg sorbent weight). Each well was equilibrated with 100% ACN, followed by incubation with 100 mM triethylammonium acetate and 95% ACN, 1% TFA in water, respectively. Samples were transferred to designated wells and were allowed to flow through. Each well was washed with a solution of 95% ACN, 1% TFA in water, and samples were eluted with 50% ACN, 0.1% TFA in water. The concentration of each sample was determined by NanoDrop (Thermo Scientific). Samples were concentrated by SpeedVac (Thermo Scientific) and reconstituted with 0.1% FA in water. Samples were transferred to vials for LC-MS/MS analysis using an Orbitrap Exploris 480 instrument (Thermo Fisher Scientific).

Tryptic glycopeptides of commonly abundant plasma glycoproteins were identified using high resolution mass spectrometry and analyzing collision induced fragmentation. The oxonium ion mass spectra from the fragmentation of glycopeptides were analyzed to determine the fucose orientation. Based on the mass of the various breakdown fragments of the glycopeptide-derived glycans, a determination was made if the fucose was a “core” fucose, “antennary” fucose (also referred to as an outer-arm fucose), “mixed” (i.e. containing a mixture of both “core” and “antennary” fucose), or “not-identified”.

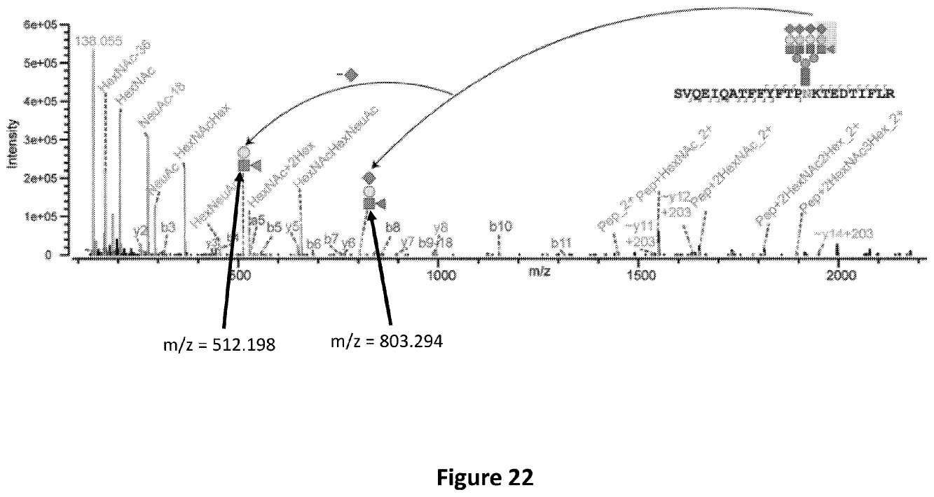

FIG. 22 is a representative figure of a mass spectra with m/z represented on the X-axis and intensity (and therefore abundance) represented on the Y-axis. Arrows indicate the breakdown products indicating the fucose is on the outer-arm (purple diamond—sialic acid, yellow circle—galactose, blue square—N-acetylglucosamine, red triangle—fucose, green circle—mannose). It is worth noting that there is a 4 glycan breakdown fragment composed of sialic acid, galactose, N-acetylglucosamine, and fucose (m/z value of 803.294). In the 4 glycan breakdown fragment, the sialic acid is connected to galactose, galactose is connected to N-acetylglucosamine, and N-acetylglucosamine is connected to fucose. The 4 glycan breakdown fragment represents a single antennary branch having a fucose in an outer arm fucose position where the aggregate glycan was cleaved at a linkage between a mannose and a N-acetylglucosamine.

Referring back to FIG. 22, there is another breakdown fragment that has 3 glycans that is the child of the 4 glycan breakdown fragment with a sialic acid removed. The 3 glycan breakdown fragment includes galactose, N-acetylglucosamine, and fucose (m/z value of 512.198). In the 3 glycan breakdown fragment, the galactose is connected to N-acetylglucosamine, and N-acetylglucosamine is connected to fucose. The presence of the 4 glycan breakdown fragment and the 3 glycan breakdown fragment as shown in FIG. 22 indicates the presence of outer arm fucosylation. In addition, it should also be noted that no fragments consistent with a core fucose orientation were measured.

Referring back to Table 11C, the SEQ ID NOS. 131, 137, 143, 155, 158, 159, 162, 166, and 171 correspond to glycopeptides that each have a non-zero coefficient along with one fucose. Using high resolution mass spectroscopy, it was determined that SEQ ID NO 131, 137, 143, 155, 159, 162, 166, and 171 each correspond to a glycoepeptide having an outer arm fucosylation format. In various embodiments, glycopeptide biomarkers with outer arm fucosylation can provide better prediction of ovarian cancer disease states.

The subset of markers that had predominantly tri and tetraantennary glycopeptides from Table 3D were used to train a regularized regression model (e.g., LASSO regression model). Coefficients for the regularized regression model (e.g., LASSO regression model) are provided in Table 11D. Using the values of Table 11D, a probability for one of the states can be determined by summing together the product of the concentration of each biomarker in the sample and the respective coefficient (of one column) and then adding the summation and the intercept to yield the logit of a probability score (see equation 1).

Table 11D. Coefficients for Peptide Structure Markers for a Regression Model to distinguish between late stage (3/4) EOC and early stage EOC (1/2) based on 12 of the biomarkers of

TABLE 3D

| SEQ ID NO | PS-Name | Coefficients |
|---|---|---|
| 131 | AACT_106_7614 | −0.46937 |
| 132 | AACT_271_6502 | −0.85484 |
| 133 | AACT_271_6503 | −0.37872 |
| 134 | AGP12_56_6503 | −0.84973 |
| 137 | AGP12_72MC_7613 | −1.12403 |
| 139 | AGP12_72_6503 | −0.99282 |
| 140 | AGP12_72_7603 | 0.580614 |
| 143 | AGP1_103_6513 | 0.844199 |
| 151 | AGP1_93_7612 | 0.221193 |
| 165 | HEMO_187_6513 | −0.78534 |
| 166 | HPT_184_6513 | 0.232181 |
| 167 | HPT_207_11904 | 0.273793 |

Using the model coefficients of Table 11D, a predicted probability can be generated for early stage and late stage ovarian cancer samples showing a stratification in predicted probabilities between the two cohorts. In various embodiments, predicted probability can be generated for classifying early stage and late stage ovarian cancer samples using the markers with non-zero coefficients. A logistic regression model was used with a subset of the glycopeptides of Table 3D where the glycopeptides had 1 or more sialic acids and zero or more fucosylations for the early and late stage EOC cohorts. The glycopeptides that included fucose were found to be associated with EOC. In addition, glycopeptides that included fucose and also carrying tri- and tetra-antennary glycan structure were found to be more strongly associated with EOC.

Table 15 shows the accuracy, sensitivity, and specificity for the training data set and the testing data set. The relative performance of Table 15 is better than Table 14 indicating that the subset of biomarkers using predominantly tri and tetra antennary glycans generated a better model for determining early vs late stage EOC.

TABLE 15

| | Train | Test |
|---|---|---|
| Accuracy | 0.902 | 0.85 |
| Sensitivity | 1 | 0.882 |
| Specificity | 0.5 | 0.667 |

### VIII.B. Exemplary Retrospective & Prospective Analysis

A validation study was conducted using both retrospective patient samples and samples collected prospectively in the ongoing Clinical Validation of the InterVenn Ovarian CAncer Liquid Biopsy (VOCAL) study. Samples included those from patients with malignant EOC and patients with benign pelvic tumors. Samples were processed in a manner similar to the manner described for the Exemplary Retrospective Analysis in Section VII.A above.

A logistic regression model was built identifying a panel of 38 peptide structures (same as those in Table 3 above). This panel of 38 peptide structures had an overall predictive accuracy of over 86% for the prediction of malignancy versus benign status of pelvic tumors.

Table 10 below provides the fold changes and p-values for the 38 peptide structures also identified in Table 3 above based on differential expression analysis (DEA). These peptide structures are ordered both in Table 3 and in Table 10 with respect to relative significance to the probability score generated by the model based on p-values. In this context, more significant peptide structures have lower p-values, while less significant peptide structures have higher p-values. In other words, relative significance to the probability score decreased with increasing p-values.

### IX. Additional Considerations

Any headers and/or sub-headers between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

What is claimed is:

1. A method for diagnosing a subject with respect to an ovarian cancer disease state, the method comprising:
receiving peptide structure data corresponding to a biological sample obtained from the subject;
analyzing the peptide structure data using a supervised machine learning model to generate a disease indicator that indicates whether the biological sample evidences the ovarian cancer disease state of having early stage or late stage ovarian cancer based on at least one peptide structure selected from one of a group of peptide structures identified in Tables 3B, 3C, or 3D,
generating a diagnosis output based on the disease indicator.

2. The method of claim 1, wherein the disease indicator comprises a score.

3. The method of claim 2, wherein generating the diagnosis output comprises:
determining that the score falls above a selected threshold; and
generating the diagnosis output based on the score falling above the selected threshold, wherein the diagnosis output includes a classification of late stage ovarian cancer disease state.

4. The method of claim 2, wherein generating the diagnosis output comprises:
determining that the score falls below a selected threshold; and
generating the diagnosis output based on the score falling below the selected threshold, wherein the diagnosis output includes a classification of early stage ovarian cancer disease state.

5. The method of claim 3 or claim 4, wherein the score comprises a probability score and the selected threshold is 0.5.

6. The method of claim 3 or claim 4, wherein the selected threshold falls within a range between 0.30 and 0.65.

7. The method of any one of claims 1-6, wherein analyzing the peptide structure data comprises:
analyzing the peptide structure data using a binary classification model.

8. The method of any one of claims 1-7, wherein a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 126-175 in Table 3D as defined in Table 5.

9. The method of any one of claims 1-8, further comprising:
training the supervised machine learning model using training data,
wherein the training data comprises a plurality of peptide structure profiles for a plurality of subjects and a plurality of subject diagnoses for the plurality of subjects, wherein the plurality of subject diagnoses includes a diagnosis for any subject of the plurality of subjects determined to have early stage or late stage ovarian cancer.

10. The method of any one of claims 1-9, further comprising:
performing a differential expression analysis using initial training data to compare a first portion of the plurality of subjects diagnosed with the classification of early stage ovarian cancer disease state versus a second portion of the plurality of subjects diagnosed with the classification of late stage ovarian cancer disease state; and
identifying a training group of peptide structures based on the differential expression analysis for use as prognostic markers for the ovarian cancer disease state; and
forming the training data based on the training group of peptide structures identified.

11. The method of claim 9, wherein training the supervised machine learning model comprises reducing the training group of peptide structures to a final group of peptide structures identified in Tables 3B, 3C, or 3D.

12. The method of any one of claims 9-11, wherein each peptide structure profile of the plurality of peptide structure profiles includes a feature selected from one of a relative abundance and a concentration for a corresponding peptide structure.

13. The method of any one of claims 9-12, wherein the plurality of peptide structure profiles includes a first peptide structure profile with a relative abundance for a corresponding peptide structure and a second peptide structure profile with a concentration for the corresponding peptide structure.

14. The method of any one of claims 1-13, wherein the supervised machine learning model comprises a logistic regression model.

15. The method of any one of claims 1-14, wherein the first group of peptide structures in Table 3B, 3C, or 3D is used to distinguish between the ovarian cancer disease state being late stage or early stage.

16. The method of any one of claims 1-15, wherein the quantification data for a peptide structure of the set of peptide structures comprises at least one of an abundance, a relative abundance, a normalized abundance, a relative quantity, an adjusted quantity, a normalized quantity, a relative concentration, an adjusted concentration, or a normalized concentration.

17. The method of any one of claims 1-16, wherein the peptide structure data is generated using multiple reaction monitoring mass spectrometry (MRM-MS), wherein the using of the MRM-MS comprises:
ionizing one or more glycopeptides to form ionized glycopeptides;
filtering the ionized glycopeptides with a mass filter to form filtered glycopeptides;
fragmenting the filtered glycopeptides in a collision chamber into product ions; and
detecting the product ions.

18. The method of any one of claims 1-17, further comprising:
preparing a sample of the biological sample using reduction, alkylation, and enzymatic digestion to form a prepared sample that includes a set of peptide structures.

19. The method of claims 1-18, wherein the subject has already been diagnosed as having ovarian cancer.

20. The method of any one of claims 1-19, wherein generating the diagnosis output comprises:
generating a report identifying that the biological sample evidences the early stage or late stage ovarian cancer disease state.

21. The method of any one of claims 1-20, further comprising:
generating a treatment output based on at least one of the diagnosis output or the disease indicator.

22. The method of claim 21, wherein the treatment output comprises at least one of an identification of a treatment to treat the subject or a treatment plan.

23. The method of claim 22, wherein the treatment comprises at least one of surgery, radiation therapy, a targeted drug therapy, chemotherapy, immunotherapy, hormone therapy, or neoadjuvant therapy.

24. The method of any one of claims 1-23, wherein the group of peptide structures in Tables 3B, 3C, or 3D is listed in order of relative significance to the disease indicator.

25. The method of any one of claims 1-24, further comprising:
preparing a sample of the biological sample using reduction, alkylation, and enzymatic digestion to form a prepared sample that includes a set of peptide structures.

26. The method of claim 25, further comprising:
generating the peptide structure data from the prepared sample using multiple reaction monitoring mass spectrometry (MRM-MS).

27. A method of training a model to diagnose a subject with respect to an ovarian cancer disease state being either early stage or late stage, the method comprising:
receiving quantification data for a panel of peptide structures for a plurality of samples for a plurality of subjects,
wherein the plurality of subjects includes a first portion diagnosed with a classification of the early stage ovarian cancer disease state and a second portion diagnosed with a classification of the late stage ovarian cancer disease state;
wherein the quantification data comprises a plurality of peptide structure profiles for the plurality of subjects; and
training a machine learning model using the quantification data to diagnose a biological sample with respect to the ovarian cancer disease state using a group of peptide structures associated with the ovarian cancer disease state, wherein the group of peptide structures is identified in Table 3B, 3C, or 3D.

28. The method of claim 27, wherein the machine learning model comprises a logistic regression model.

29. The method of claim 28, wherein the logistic regression model comprises a LASSO regression model.

30. The method of any one of claims 27-28, further comprising:
identifying an initial plurality of peptide structure profiles;
filtering the initial plurality of peptide structure profiles by a coefficient of variation to generate a plurality of peptide structure profiles for use in training the machine learning model.

31. The method of claim 30, wherein the filtering is performed to exclude peptide structure profiles having the coefficient of variation at or above 20%.

32. The method of claim 30, wherein training the machine learning model comprises reducing the plurality of peptide structure profiles using LASSO regression to identify a final group of peptide structures identified in Table 3B, 3C, or 3D.

33. The method of any one of claims 27-32, wherein the quantification data for the panel of peptide structures for the plurality of subjects diagnosed with the plurality of ovarian cancer disease states comprises at least one of an abundance, a relative abundance, a normalized abundance, a relative quantity, an adjusted quantity, a normalized quantity, a relative concentration, an adjusted concentration, or a normalized concentration.

34. The method of any one of claims 27-32, wherein the trained model uses a relative abundance for a first portion of the first group of peptide structures and a concentration for a second portion of the second group of peptide structures.

35. The method of any one of claims 27-34, wherein each peptide structure profile of the plurality of peptide structure profiles includes a feature selected from one of a relative abundance and a concentration for a corresponding peptide structure.

36. The method of any one of claims 27-35, wherein the plurality of peptide structure profiles includes a first peptide structure profile with a relative abundance for a corresponding peptide structure and a second peptide structure profile with a concentration for the corresponding peptide structure.

37. A composition comprising at least one of peptide structures identified in Table 3B, 3C, or 3D.

38. A method for diagnosing a subject with respect to an ovarian cancer disease state, the method comprising:
analyzing the peptide structure data using a supervised machine learning model to generate a disease indicator that indicates whether a biological sample evidences the ovarian cancer disease state of having early stage or late stage ovarian cancer based on a group of glycopeptide structures, the group of glycopeptide structures comprising tri-antennary or tetra-antennary sialic acid moieties, wherein a portion of the glycopeptide structures of the group are fucosylated;
generating a diagnosis output based on the disease indicator.

39. The method of claim 38, wherein the group of glycopeptide structures comprises at least one glycopeptide structure identified in Tables 3B, 3C, or 3D.

40. The method of claim 38, wherein the group of glycopeptide structures comprises at least three glycopeptide structures identified in Tables 3B, 3C, or 3D.

41. The method of claim 38, wherein the group of glycopeptide structures comprises at least five glycopeptide structures identified in Tables 3B, 3C, or 3D.

42. The method of claim 38, wherein the group of glycopeptide structures comprises at least ten glycopeptide structures identified in Tables 3B, 3C, or 3D.

43. The method of claim 38, wherein the peptide structure data was generated with a mass spectrometer using the biological sample obtained from the subject.

44. The method of any one of claims 38-43 further comprising:
preparing a sample of the biological sample using reduction, alkylation, and enzymatic digestion to form a prepared sample that includes a set of peptide structures.

45. The method of claim 44, further comprising:
generating the peptide structure data from the prepared sample using multiple reaction monitoring mass spectrometry (MRM-MS).

46. The method of any one of claims 38-45, wherein the peptide structure data is generated using multiple reaction monitoring mass spectrometry (MRM-MS), wherein the using of the MRM-MS comprises:
ionizing one or more glycopeptides to form ionized glycopeptides;
filtering the ionized glycopeptides with a mass filter to form filtered glycopeptides;
fragmenting the filtered glycopeptides in a collision chamber into product ions; and
detecting the product ions.

47. The method of any claim 17 or 24, wherein the peptide structure data is listed in Table 3D and the detected product ion comprises a first product having a m/z value listed in Table 4C.

48. The method of any one of claims 1-7, wherein a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 130-135, 137, 139, 140, 143, 148, 149, 155, 158-162, 166, and 171 in Table 3D as defined in Table 5.

49. The method of any one of claims 1-7, wherein a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 130-135, 137, 139, 140, 143, 148, 149, 155, 159-162, 166, and 171 in Table 3D as defined in Table 5.

50. The method of claim 49, wherein the glycan structure, corresponding to the peptide sequence of SEQ ID NOS: 131, 137, 143, 155, 159, 162, 166, and 171, includes a fucose and the fucose is in an outer arm orientation.

51. The method of any one of claims 1-7, wherein a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 131, 137, 143, 155, 159, 162, 166, and 171 in Table 3D as defined in Table 5, wherein a fucose of the glycan structure comprises an outer arm orientation.

52. The method of any one of claim 50 or 51, wherein the outer arm orientation of the fucose comprises the fucose being linked to a N-acetylglucosamine by a α-(1-3/4) linkage.

53. The method of any one of claims 1-7, wherein the at least one peptide structure selected from one of a group of peptide structures is identified in Tables 3D.

54. The method of any one of claims 1-7, wherein a peptide structure of the at least one peptide structure comprises a glycopeptide structure defined by a peptide sequence and a glycan structure linked to the peptide sequence at a linking site of the peptide sequence, as identified in Table 3D, with the peptide sequence being one of SEQ ID NOS: 131-134, 137, 139, 140, 143, 151, 165-167 in Table 3D as defined in Table 5.

55. The method of claim 49, wherein the glycan structure, corresponding to the peptide sequence of SEQ ID NOS: 131, 137, and 143, includes a fucose and the fucose is in an outer arm orientation.

56. The method of claim 55, wherein the outer arm orientation of the fucose comprises the fucose being linked to a N-acetylglucosamine by a α-(1-3/4) linkage.

57. A method of treating ovarian cancer in an individual, the method comprising: administering to the individual an ovarian cancer therapy, wherein the individual has been determined to be responsive to the ovarian cancer therapy via a trained machine learning classifier that distinguishes between responsive and non-responsive individuals who have received the ovarian cancer therapy, based at least in part on a group of peptide structures identified in Tables 3B, 3C, or 3D.
